(19) United States
(12) Patent Application Publication
(10) Pub. No.: US 2005/0170483 A1
(43) Pub. Date: Aug. 4, 2005

(54) Chaotic fermentation of ethanol

(76) Inventors: Said S.E.H. Elnashaie, Auburn, AL (US); Parag Garhyan, Kalamazoo, MI (US)

Correspondence Address: GARDNER GROFF, P.C., 2018 POWERS FERRY ROAD, SUITE 800, ATLANTA, GA, 30339, US

(21) Appl. No.: 10/978,293

(22) Filed: Oct. 29, 2004

Related U.S. Application Data
(60) Provisional application No. 60/515,262, filed on Oct. 29, 2003.

(51) Int. Cl. C12P 7/06; C12P 19/02; G06F 19/00; G01N 33/48; G01N 33/50

(52) U.S. Cl. 435/161; 435/105; 702/19

(57) ABSTRACT

A method and apparatus for fermentation of ethanol. A method comprises selecting a desired fermentation on process with oscillatory process characteristics, providing a fermentor and a biocatalyst, feeding a substrate to the fermentor, and fermenting under chaotic conditions. An apparatus comprises a fermentor, a process control system capable of operating the fermentor under chaotic conditions, and a membrane selective for ethanol. The invention can be applied to other catalytic processes.

### CROSS REFERENCE TO RELATED APPLICATIONS

This application claims, priority to U.S. Provisional Application Ser. No. 60/515,262, filed Oct. 29, 2003, hereby incorporated by reference in its entirety for all of its teachings.

### BACKGROUND

There are many bioreactor processes being used today of commercial significance. One such process is fermentation. Fermentation is used in many industries, including the production of ethanol.

Ethanol is used for many things, for example, in gasoline formulations for octane enhancement and as an oxygenate for the control of automotive tailpipe emissions. C. E. Wyman, “Ethanol from lignocellulosic biomass: technology, economics, and opportunities,” Bioresour. Technol. 50(1), 3-15 (1994); K. T. Knapp, F. D. Stump and S. B. Tejada. “The effect of ethanol fuel on the emissions of vehicles over a wide range of temperatures,” J. Air Waste Manage. Assoc. 48(7), 646-653 (1998); W. D. Hsieh, R. H. Chen, T. L. Wu and T. H. Lin, “Engine performance and pollutant emission of an SI engine using ethanol-gasoline blended fuels,” Atmos. Environ. 36(3), 403-410 (2002). Due to high feedstock prices (approximately 90% of ethanol is produced from corn, and this utilizes about 6.2% of the total corn crop) for production of ethanol and competition from other products for its gasoline uses, it is desirable to make the process of ethanol production more economical.

One of the major problems for the efficient production of ethanol is the product (ethanol) inhibition of the biocatalyzing microorganism. One approach to process improvement would be using a continuous fermentation integrating an ethanol removing/recovery operation, thereby maintaining the ethanol concentration in the fermentation broth at a level which is minimally inhibitory to fermenting organisms.

Attempts to address the high feedstock price issue have included use of less expensive feed stocks. Cellulosic biomass (agricultural waste/residue etc.) can be used for conversion to ethanol as a less expensive feedstock alternative to corn. The basic steps of ethanol production from cellulose are
1. hydrolysis of biomass to sugars and
2. fermentation of sugars to ethanol.

There are several places in the process where there are bottlenecks for efficient production of ethanol from these less expensive cellulosic wastes. These include:
inhibitory effect of ethanol on microorganisms (inhibition due to changes in fluidity of biological membranes),
substrate inhibition at high sugar concentrations due to saturation,
inefficient fermentation of difficult sugars with conventional microorganisms,
limitation on flow rate for continuous process, and
instability, bifurcation, and chaotic behavior in the high sugar concentration range.

There remains a need for improving production of ethanol by addressing bottlenecks.

### SUMMARY OF THE INVENTION

The invention includes a method for producing a fermented product comprising selecting a desired fermentation process with oscillatory process characteristics, a fermentor and a biocatalyst, a method for feeding a substrate to the fermentor and fermenting under chaotic conditions to produce the fermented product, in which the biocatalyst and substrate correspond to the desired fermentation process.

The invention also includes a method for producing a product using a bioreactor comprising selecting a desired biochemical process with oscillatory process characteristics, and feeding a substrate to the bioreactor, providing a bioreactor and a biocatalyst, and operating the bioreactor under chaotic conditions to produce the product, in which the biocatalyst and substrate correspond to the biochemical process.

The invention also includes an improved method for fermentation of ethanol wherein the improvement comprises operating the fermentor under chaotic conditions.

The invention also includes a method for increasing efficiency and yield of an ethanol fermentation process relative to the same ethanol fermentation process operated at steady state conditions comprising selecting an ethanol fermentation process with oscillatory process characteristics, feeding a suitable substrate to the ferementor, providing a fermentor and a biocatalyst suitable for ethanol fermentation, and fermenting under chaotic conditions.

The invention also includes an apparatus for fermenting ethanol comprising a fermentor, a process control system capable of operating the fermentor under chaotic conditions, and a membrane selective for ethanol.

Existing commercial fermentation processes are not cost effective to ferment all forms of sugars used in ethanol production. A process of the present invention addresses the fundamental challenges in the development of an efficient process by using chaotic fermentation.

Current fermentor technologies are based on the assumption that steady-state operations are the most efficient and highest yielding. However, mathematical modeling has indicated that much greater ethanol yields are possible using chaotic operating conditions. These modeling predictions have been confirmed via experimental results. The current technology optimizes the yield by controlling the fermentation process with fuzzy control system that could be incorporated into a software package, for example.

In addition, a pervaporation membrane separation can be employed by this invention to further enhance the productivity of ethanol fermentation. The resulting increase in yield can reach 100%, generating a cost reduction approaching 50%. Any reactor configuration (e.g., continuous, stirred tank) that allows controlled oscillations can benefit from this chaotic processing of ethanol. The membrane separation technology is used in a manner to make an unstable environment “stable.”

The invention includes a chaotic ethanol fermentor that improves the fermentation process performance of hard-to-ferment sugars produced from hydrolysis of biomass, increasing ethanol production by about 100 percent. The technology can be applied to any CSTR fermentation process that has oscillatory process characteristics. The invention is most valuable to processes where microorganism efficiency is hindered by high concentration of the fermented product.

A process of the current invention is efficient. An embodiment of the invention achieved
about 100% improvement in fermentation yield in a dynamic and continuous ethanol production process (with the use of a membrane); similar gains may be expected in other processes and
an estimated 50% cost reduction over steady-state fermentation.

A process of the current invention is flexible, for example, it
works with an oscillatory CSTR fermentation process
allows use of a wide range of microorganisms to ferment a broad range of sugars and
integrates easily into current plant or operation methods.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows a simplified schematic diagram of the fermentor showing all of the concentrations and flow rates. FIGS. 1A and 1B show the schematic diagrams of the fermentor and in-situ ethanol removal membrane module setup with all the flow rates and concentrations shown.

FIG. 2 shows, for the first round modeling, A) Two parameter continuation diagram of ^{C}_{SO} vs D, [symbol] loci of HB points, [symbol] loci of SLP. B) Enlargement of box of Figure A.

FIG. 3 shows a comparison of experimental and simulation results from Jobses et al., 1986a): [symbol] measured ethanol concentration, [symbol] simulated ethanol concentration.

FIG. 4 shows, for the first round modeling, bifurcation diagrams at C_{SO}=140 kg/m³ with D as bifurcation parameter. Steady state branch ( [symbol] stable, [symbol] unstable); Periodic branch (••••• stable, [symbol] unstable, ♦♦♦♦♦ average of oscillations).

FIG. 5 shows, for the first round modeling, period change with D at C_{SO}=140 kg/m³.

FIG. 6 shows, for the first round modeling, unequal excursion of oscillations around the unsteady state: A) Periodic attractor at C_{SO}=140 kg/m³ and D=0.02 hr⁻¹ and B) Chaotic attractor at C_{SO}=200 kg/m³ and D=0.045842 hr⁻¹.

FIG. 7 shows, for the first round modeling, bifurcation diagrams at C_{SO}=149 kg/m³ with D as bifurcation parameter. Steady state branch ( [symbol] stable, [symbol] unstable); Periodic branch (••••• stable, [symbol] unstable, ♦♦♦♦♦ average of oscillations).

FIG. 8 shows, for the first round modeling, A) one-dimensional Poincaré bifurcation diagram (C_{SO}−D) at C_{SO}=149 kg/m³ and B) Period change with D at C_{SO}=149 kg/m³.

FIG. 9 shows, for the first round modeling, bifurcation diagrams at C_{SO}=150.3 kg/m³ with D as bifurcation parameter. Steady state branch ( [symbol] stable, [symbol] unstable); Periodic branch (••••• stable, [symbol] unstable, ♦♦♦♦♦ average of oscillations).

FIG. 10 shows, for the first round modeling, one-dimensional Poincaré bifurcation diagram at C_{SO}=150.3 kg/m³.

FIG. 11 shows, for the first round modeling, bifurcation diagrams at C_{SO}=155 kg/m³ with D as bifurcation parameter. Steady state branch ( [symbol] stable, [symbol] unstable); Periodic branch (••••• stable, [symbol] unstable, ♦♦♦♦♦ average of oscillations).

FIG. 12 shows, for the first round modeling, dynamic characteristics at C_{SO}=155 kg/m³ and D=0.04376 hr⁻¹. A) One-dimensional Poincaré bifurcation diagram; B) Enlargement of (A); C) Return point histogram.

FIG. 13 shows, for the first round modeling, bifurcation diagrams at C_{SO}=200 kg/m³ with D as bifurcation parameter. Steady state branch ( [symbol] stable, [symbol] unstable); Periodic branch (••••• stable, [symbol] unstable, ♦♦♦♦♦ average of oscillations).

FIG. 14 shows, for the first round modeling, dynamic characteristics at C_{SO}=200 kg/m³ and D=0.04584 hr⁻¹. A) One-dimensional Poincaré bifurcation diagram; B) Enlargement of chaos region of (A); C) Return point histogram.

FIG. 15 shows, for the first round modeling, bifurcation diagrams at D=0.05 hr⁻¹ with C_{SO} as bifurcation parameter. Steady state branch ( [symbol] stable, [symbol] unstable); Periodic branch (••••• stable, [symbol] unstable, ♦♦♦♦♦ average of oscillations).

FIG. 16 shows, for the first round modeling, bifurcation diagrams at D=0.045 hr⁻¹ with C_{SO} as bifurcation parameter. Steady state branch ( [symbol] stable, [symbol] unstable); Periodic branch (••••• stable, [symbol] unstable, ♦♦♦♦♦ average of oscillations).

FIG. 17 shows, for the first round modeling, A) one-dimensional Poincaré bifurcation diagram (C_{S} VS C_{SO}) at D=0.045 hr⁻¹; B) Enlargement of (A).

FIG. 18 shows, for the second round modeling, A) Two parameter continuation diagram of C_{SO} VS Dᵢₙ ( [symbol] =loci of HB points, [symbol] =loci of SLP). B) Enlargement of box of FIG. 18A.

FIG. 19 shows, for the second round modeling, bifurcation diagrams at C_{SO}=140 kg/m³ with Dᵢₙ as bifurcation parameter. Steady state branch ( [symbol] stable, [symbol] unstable); periodic branch (••••• stable, [symbol] unstable, ♦♦♦♦♦ average of oscillations).

FIG. 20 shows, for the second round modeling, period change with Dᵢₙ at C_{SO}=140 kg/m³.

FIG. 21 shows, for the second round modeling, bifurcation diagrams at C_{SO}=200 kg/m³ with Dᵢₙ as bifurcation parameter. Steady state branch ( [symbol] stable, [symbol] unstable); periodic branch (••••• stable, [symbol] unstable, ♦♦♦♦♦ average of oscillations).

FIG. 22 shows, for the second round modeling, bistability at C_{SO}=200 kg/m³ and Dᵢₙ=1.50 hr⁻¹ (H is the high conversion steady state and L is the low conversion steady state).

FIG. 23 shows, for the second round modeling, dynamic characteristics at C_{SO}=200kg/m³and Dᵢₙ=0.04584 hr⁻¹ A) One-dimensional Poincaré bifurcation diagram; B) Enlargement of chaos region of (A); C) Return point histogram.

FIG. 24 shows, for the second round modeling, bifurcation diagrams at Dᵢₙ=0.05 hr⁻¹ with C_{SO} as bifurcation parameter. Steady state branch ( [symbol] stable, [symbol] unstable); periodic branch (••••• stable, [symbol] unstable, ♦♦♦♦♦ average of oscillations).

FIG. 25 shows, for the second round modeling, bifurcation diagrams at Dᵢₙ=0.045 hr⁻¹ with C_{SO} as bifurcation parameter. Steady state branch ( [symbol] stable, [symbol] unstable); Periodic branch (••••• stable, [symbol] unstable, ♦♦♦♦♦ average of oscillations).

FIG. 26 shows, for the second round modeling, one-dimensional Poincaré bifurcation diagram (C_{S} vs C_{SO}) at Dᵢₙ=0.045 hr⁻¹.

FIG. 27 shows, for the second round modeling, bifurcation diagrams at C_{SO}=140 kg/m³ and Dᵢₙ=0.02 hr⁻¹ with A_{M} as bifurcation parameter. Steady state branch ( [symbol] stable, [symbol] unstable); Periodic branch (••••• stable, [symbol] unstable, ♦♦♦♦♦ average of oscillations).

FIG. 28 shows, for the second round modeling, bifurcation diagrams at C_{SO}=200 kg/m³ and Dᵢₙ=0.04584 hr⁻¹ with A_{M} as bifurcation parameter. Steady state branch ( [symbol] stable, [symbol] unstable); Periodic branch (••••• stable, [symbol] unstable, ♦♦♦♦♦ average of oscillations).

FIG. 29 shows, for the second round modeling, one-dimensional Poincaré bifurcation diagram (C_{S} vs A_{M}) at C_{SO}=200 kg/m³ and Dᵢₙ=0.04584 hr⁻¹.

FIG. 30 shows a bifurcation diagram for C_{SO}=140 g/L and D as the bifurcation parameter from the Examples.

FIG. 31 shows bifurcation diagrams for C_{SO}=200 g/L and D as the bifurcation parameter from the Examples. A) D=0.25 hr⁻¹; B) D=0.045 hr⁻¹.

FIG. 32 shows a simplified schematic of the experimental setup from the Examples.

FIG. 33 shows the results of the batch experiment of glucose fermentation with Z. mobilis from the Examples.

FIG. 34 shows a comparison of simulated and experimental ethanol concentrations in continuous operation mode for C_{SO}=140 g/L at D=0.022 hr⁻¹ (corresponding to case 1) from the Examples.

FIG. 35 shows a comparison of simulated and experimental ethanol concentrations in continuous operation mode for C_{SO}=140 g/L at D=0.04 hr⁻¹ (corresponding to case 1) from the Examples.

FIG. 36 shows a comparison of simulated and experimental ethanol concentrations in continuous operation mode for C_{SO}=140 g/L at D=0.06 hr⁻¹ (corresponding to case 1) from the Examples.

FIG. 37 shows the results of experiments for C_{SO}=200 g/L at D=0.25 hr⁻¹ (leading to the high-ethanol-concentration branch in case 2) from the Examples.

FIG. 38 shows the results of experiments for C_{SO}=200 g/L at D=0.25 hr⁻¹ (leading to the low-ethanol-concentration branch in case 2) from the Examples.

FIG. 39 shows results of experiments for C_{SO}=200 g/L at D=0.045 hr⁻¹ (leading to the stable branch in case 2) from the Examples.

### DETAILED DESCRIPTION

Before the present compounds, compositions, articles, devices, and/or methods are disclosed and described, it is to be understood that the aspects described below are not limited to specific synthetic methods, specific methods as such may, of course, vary. It is also to be understood that the terminology used herein is for the purpose of describing particular aspects only and is not intended to be limiting.

In this specification and in the claims which follow, reference will be made to a number of terms which shall be defined to have the following meanings:

It must be noted that, as used in the specification and the appended claims, the singular forms “a,” “an,” and “the” include plural referents unless the context clearly dictates otherwise. Thus, for example, reference to “an enzyme” includes mixtures of enzymes, reference to “a microorganism” includes mixtures of two or more such microorganisms, and the like.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Existing commercial fermentation processes are not cost effective to ferment all forms of sugars used in ethanol production. A process of the present invention can address the fundamental challenges in the development of an efficient process by using chaotic fermentation.

Current conventional fermentor technologies are based on the assumption that steady-state operations are the most efficient and highest yielding. However, mathematical modeling has indicated that much greater ethanol yields are possible using chaotic operating conditions (discussed further below). These modeling predictions have been confirmed via experimental results. See Examples. The current technology can optimize the yield by controlling the fermentation process with fuzzy control system that could be incorporated into a software package, for example.

In addition, a pervaporation membrane separation can be employed as part of a process this invention to further enhance the productivity of ethanol fermentation. The resulting increase in yield can reach about 100%, generating a cost reduction approaching 50%. Any continuous, stirred tank configuration that allows controlled oscillations can benefit from this chaotic processing of ethanol. The membrane separation technology is used in a manner to make an unstable environment “stable.”

The invention includes a chaotic ethanol fermentor that improves the fermentation process performance of hard-to-ferment sugars, such as those produced from hydrolysis of biomass, increasing ethanol production by about 100 percent. The technology can be applied to any CSTR fermentation process that has oscillatory process characteristics. The invention is most valuable to processes where microorganism efficiency is hindered by high concentration of the fermented product.

A process of the present invention is efficient. An embodiment of the invention achieved
about 100% improvement in fermentation yield in a dynamic and continuous ethanol production process (with the use of a membrane); similar gains may be expected in other processes and
an estimated 50% cost reduction over steady-state fermentation.

A process of the present invention is flexible, for example, it can
work with an oscillatory CSTR fermentation process
allow use of a wide range of microorganisms to ferment a broad range of sugars and
integrate easily into current plant or operation methods.

In order to develop the improved process of the present invention, mathematical modeling of an example system was performed. Experimental verification followed the modeling. Both are discussed below.

Modeling

An experimentally-verified model was used to explore the conditions for increasing productivity and yield of the ethanol fermentation process. Detailed bifurcation analysis was carried out to uncover the rich static and dynamic behavior of the fermentor with/without ethanol removal membranes. The emphasis was on producing higher ethanol yield and productivity through unconventional modes of operation. Possible increase of sugar conversion and ethanol productivity using periodic and chaotic operation at high sugar concentrations was investigated for continuous stirred tank fermentors with/without ethanol removal membranes.

Modeling Background

A quantitative knowledge of bioculture stability and dynamics is often required to understand, control, and optimize a process. Davey, H. M., Davey, C. L., Woodward, A. M., Edmonds, A. N., Lee, A. W., and Kell, D. B. (1996). “Oscillatory, stochastic and chaotic growth rate fluctuations in permittistatically controlled yeast cultures,” Biosystems, 39(1), 43-61; Wolf, J., Sohn, H. Y., Heinrich, R., and Kuriyama, H. (2001). “Mathematical analysis of a mechanism for autonomous metabolic oscillations in continuous culture of Saccharomyces cerevisiae,” FEBS Lett., 499(3), 230-234. Quantitative knowledge of systems is often explored by modeling.

In fermentation processes, many investigators have reported the presence of sustained oscillations in experimental fermentors (especially at high sugar concentrations), and they have developed suitable mathematical relations to model these fermentors. Jarzebski, A. B. (1992). “Modeling of oscillatory behavior in continuous ethanol fermentation,” Biotech. Lett., 14(2), 137-142; Ghommidh, C., Vaija, J., Bolarinwa, S. and Navarro, J. M. (1989). “Oscillatory behavior of Zymomonas mobilis in continuous cultures: a simple stochastic model,” Biotech. Lett., 2(9), 659-664; Daugulis, A. J., McLellan, P. J. and Li, J. (1997). “Experimental investigation and modeling of oscillatory behavior in the continuous culture of Zymomonas mobilis,” Biotech. & Bioeng., 56(1), 99-105; McLellan, P. J., Daugulis, A. J., and Li, J. (1999). “The incidence of oscillatory behavior in the continuous fermentation of Zymomonas mobilis,” Biotech. Prog., 15(4), 667-680; Jobses, I. M. L., Egberts, G. T. C., Ballen, A. V. and Roels, J. A. (1985). “Mathematical modeling of growth and substrate conversion of Zymomonas mobilis at 30 and 35° C.,” Biotech. & Bioeng., 27(7), 984-995; Jobses, I. M. L., Egberts, G. T. C., Luyben, K. C. A. M. and Roels, J. A. (1986a). “Fermentation kinetics of Zymomonas mobilis at high ethanol concentrations: oscillations in continuous cultures,” Biotech. & Bioeng., 28(6), 868-877; Jobses, I. M. L. (1986b). “Modeling of anaerobic microbial fermentations: the production of alcohols by Zymomonas mobilis and Clostridium beijerincki,” Ph.D. Thesis, Delft University, Delft, Holland. Xiu, Zeng and Deckwer (1998), Zamamiri, Birol and Hjortso (2001), and Zhang and Henson (2001) have carried out detailed multiplicity and stability analyses of microorganisms in continuous cultures. Xiu, Z. L., Zeng, A. P. and Deckwer, W. D. (1998). “Multiplicity and stability analysis of microorganisms in continuous culture: effects of metabolic overflow and growth inhibition,” Biotech. & Bioeng., 57(3),251-261; Zamamiri, A. M., Birol, G., and Hjortso, M. A. (2001). “Multiple steady states and hysteresis in continuous, oscillating cultures of budding yeast,” Biotech. and Bioeng., 75(3), 305-312; Zhang, Y and Henson, M. A. (2001). “Bifurcation analysis of continuous biochemical reactor models,” Biotech. Prog., 17(4), 647-660.

Multiplicity of steady states in chemically reactive systems was first observed by Liljernoth. Liljernoth, F. G. (1919) “Starting and stability phenomenon of ammonia oxidation and similar reactions,” Chem. Met. Eng., 19, 287-291. This phenomenon in chemical reactors was later expanded upon by others (e.g., Aris, R. and Amundson, N. R. (1958) “An analysis of chemical reactor stability and control, Parts I-III,” Chem. Eng. Sci., 7(3), 121-155; Balakotaiah, V. and Luss, D. (1981). “Analysis of multiplicity patterns of a CSTR,” Chem. Eng. Comm., 13(1-3), 111-132; Balakotaiah, V. and Luss, D. (1983a). “Multiplicity criteria for multiple-reaction networks,” AlChE J., 29(4), 552-560; Balakotaiah, V. and Luss, D. (1983b). “Multiplicity features of reacting systems,” Chem. Eng. Sci., 38(10), 1709-1721; Hlavacek, V and Rompay, P. V. (1981). “Current problems of multiplicity, stability and sensitivity in chemically reactive systems,” Chem. Eng. Sci., 36(10),1587-1597). This phenomenon of multiplicity is treated in the mathematical literature in more general and abstract terms under the title of “bifurcation theory” (Golubitsky, M. and Schaeffer, D. G. (1985). Singularities and bifurcation theory, Vol I. Applied Mathematical Science, Vol V, Springer, Berlin). Excellent reviews for the bifurcation behavior of chemically reactive and biochemical systems have been published by Ray (1977), Bailey (1977; 1998), Gray and Scott (1994), Elnashaie and Elshishini (1996), and Epstein and Pojman (1998). Ray, W. H. (1977). Bifurcation phenomena in chemically reacting systems. Applications of Bifurcation Theory, ed. Rabinowiz, P. H., Academic Press, New York, 285-315; Bailey, J. E. and Ollis, D. R. (1977). Biochemical Engineering Fundamentals, McGraw Hill, N.Y.; Bailey, J. E. (1998). “Mathematical modeling and analysis in biochemical engineering: past accomplishments and future opportunities,” Chem. Eng. Comm., 13(1-3), 111-132; Gray, P. and Scott, S. K. (1994). Chemical Oscillations and Instabilities. Clarendon Press, Oxford; Elnashaie, S. S. E. H., and Elshishini, S. S (1996). Dynamic Modelling, Bifurcation and Chaotic Behavior of Gas-Solid Catalytic Reactor. Gordon and Breach Publishers, London, UK; Epstein, I. R., and Pojman, J. A. (1998). An introduction to Nonlinear Chemical Dynamics. Oxford University Press, New York.

Bifurcation analysis was utilized in the modeling for development of the present invention. Bifurcation analysis is the study of how the qualitative properties of a non-linear dynamics system change as key parameters are varied. There is a change in the number of solutions of an equation as a parameter (or more) is varied. The equation may be algebraic, ordinary differential equation (ODE), partial differential equation (PDE), or difference equation. The term “solution” means static solution or periodic solution.

Mathematically, consider a continuous-time non-linear system depending on a parameter vector α:
dx/dt=f(x, α), x ∈ Rⁿ, α∈ R^{I},
where f is smooth with respect to both the state vector x and the bifurcation parameter vector α. If x_{O} is an equilibrium point where all the real parts of the eigenvalues of the Jacobian matrix Df (x_{O}) are non-zero, then a small perturbation in the model parameter will not change the qualitative behavior of the system, i.e., a stable equilibrium is attained.

Bifurcation occurs when some of the eigenvalues approach the imaginary axis in the complex plane. The simplest bifurcations are associated with a single real eigenvalue becoming equal to zero (λ₁=0) or a pair of complex conjugate eigenvalues crossing the imaginary axis (λ_{1,2}=±iwₒ, wₒ>0).

The term “attractor” is the solution at which the system settles after a long transient time, whether starting from a certain initial condition or after being exposed to some external disturbances. In general, the attractors can be point, periodic, quasi-periodic, or strange (chaotic or non-chaotic) attractors.

Systems that upon analysis are found to be non-linear, non-equilibrium, deterministic, dynamic and that incorporate randomness so that they are sensitive to initial conditions and have strange attractors are said to be “chaotic.” These are necessary but not sufficient conditions. For a system to be chaotic the “Lyapunov exponent” must be positive.

The “Lyapunov Exponent” (LE) measures the exponential separation of trajectories with time in phase space. LE is indicative of chaos because nearby points separate exponentially, i.e., they separate rapidly, which suggests instability. Positive LE=Chaotic

It is important to point out that carrying out bifurcation analysis, rather than simply producing dynamic simulations of the model equations for different parameter values and conditions, has the following advantages:

1. For a slow process like fermentation, dynamic simulation may be inefficient, inconclusive, and may not be able to locate the model characteristics that are responsible for certain rich dynamic behavior such as bifurcation and chaos.

2. Some dynamic characteristics may be completely missed or neglected as only a limited number of dynamic simulation runs can be performed.

Model

In microbial fermentation processes, biomass acts as the catalyst for substrate conversion and is also produced by the process. This is a biochemical example of autocatalysis. P. Gray, S. K. Scott, “Autocatalytic reactions in the isothermal continuous stirred tank reactor; Oscillations and instabilities in the system A+2B→3B; B→C” Chem. Eng. Sci. 39(6), 1087-1097 (1984); D. T. Lynch, “Chaotic behavior of reaction systems: consecutive quadratic/cubic autocatalysis via intermediates,” Chem. Eng. Sci. 48(11), 2103-2108 (1993); J. E. Bailey, “Mathematical modeling and analysis in biochemical engineering: past accomplishments and future opportunities,” Chem. Eng. Comm. 13(1-3), 111-132 (1998).

A base model for the biomass of the system was first chosen. Several models have been proposed to account for the oscillatory behavior of Zymomonas mobilis in an ethanol fermentor bioreactor system. The model used for modeling the biomass of the example system for the present invention was the model of Jobses etal., 1985; 1986a (Jobses, I. M. L., Egberts, G. T. C., Ballen, A. V. and Roels, J. A. (1985). Mathematical modeling of growth and substrate conversion of Zymomonas mobilis at 30 and 35° C. Biotechnology & Bioengineering, 27(7), 984-995; Jobses, I. M. L., Egberts, G. T. C., Luyben, K. C. A. M. and Roels, J. A. (1986a). Fermentation kinetics of Zymomonas mobilis at high ethanol concentrations: oscillations in continuous cultures. Biotechnology & Bioengineering, 28(6), 868-877), which is an important experimentally verified model.

Mathematical modeling of fermentation processes can be classified into two main categories namely, structured and unstructured models. In unstructured models the biomass is regarded as a chemical compound in a solution with an average formula. In structured models, biomass is regarded as a number of biochemical compounds, thus taking into consideration the change in internal composition of the organism.

The Jobses et al. model is an unsegregated-structured two-compartment representation. The model considered biomass as being divided into compartments (K-compartment and G-compartment) containing specific groupings of macromolecules (e.g., K-compartment is identified with RNA, carbohydrates, and monomers of macromolecules while the G-compartment is identified with protein, DNA, and lipids).

The effect of elevated ethanol concentration on the fermentation kinetics resembles the effect of elevating the temperature of fermentation broth (Fieschko, J. and Humphrey, A. E. (1983). “Effects of temperature and ethanol concentration on the maintenance and yield coefficient of Zymomonas mobilis,” Biotech. & Bioeng., 25(6), 1655-1660). Also, elevated temperature enlarges the inhibitory effect of ethanol (Lee, K. J., Skotnicki, M. L., Tribe, D. E. and Rogers, P. L. (1981). “The effect of temperature on the kinetics of ethanol production by strains of Zymomonas mobilis,” Biotech. Letters, 3(6), 291-296).

The oscillatory behavior of product-inhibited cultures cannot simply be described by a common inhibition term in the equation of biomass growth (Kurano, N., Kotera, S., Okazaki, M. and Miura, Y. (1984). “Oscillation of filamentous bacterium Sphaerotilus sp. in continuous culture,” J. Ferm. Tech., 62(5), 395-400; Wolf, J.; Sohn, H. Y.; Heinrich, R.; Kuriyama, H. Mathematical Analysis of a Mechanism for Autonomous Metabolic Oscillations in Continuous Culture of Saccharomyces cerevisiae. FEBS Lett. 2001, 499, 230). A better description necessitates the inclusion of an indirect (or delayed) effect of the product on the growth rate as was experimentally demonstrated by Kurano et al., 1984. Kurano et al. (1984) introduced a decay rate of μₘₐₓ caused by the accumulation of the inhibitory product pyruvic acid. Jobses (1986b) proposed a more mechanistic, structured model, in which μₘₐₓ is related to an internal key-compound (e). The inhibitory action of ethanol is realized by the inhibition of the formation of this key compound (Jobses et al. 1985; 1986a, Jobses, 1986b).

Mathematically these descriptions are equivalent, except that the key compound is washed out as a part of the biomass in continuous cultures, and the rate constant μₘₐₓ is not. The proposed indirect inhibition model provides qualitatively a good description of the experimental results. The quantitative description is, however, not optimal, as it was necessary to adapt some parameters values for the description of the oscillations at different dilution rates. A quantitatively adequate model, must probably also account for inhibition of the total fermentation (including growth rate independent metabolism) and dying off of the biomass at long contact times at high ethanol concentrations.

Jobses and coworkers (1985; 1986a; Jobses, I. M. L. (1986b). Modeling of anaerobic microbial fermentations: the production of alcohols by Zymomonas mobilis and Clostridium beijerincki. PhD Thesis, Delft University, Delft, Holland) studied the oscillatory behavior utilizing this model in which the synthesis of a cellular component “e” (which is essential for both growth and product formation) had a non-linear dependence on ethanol concentration. Hence, the inhibition by ethanol did not directly influence the specific growth rate of the culture, but its effect was indirect.

A base model for the fermentation system was chosen next. One of the most widely used models to model fermentation processes is the maintenance model (Pirt, S. J. (1965). “The maintenance energy of bacteria in growing cultures,” Proc. of the Royal Society of London, Series B: Biological Sciences, 163, 224-231), in which substrate consumption is expressed in the form:

$${r}_{s} = ( \frac{1}{{Y}_{SX}} ) {r}_{X} + {m}_{S} {C}_{X .} \qquad ( 1 )$$

The first term accounts for growth rate, and the second term accounts for the maintenance. The growth term and the maintenance factor have their classical definitions. J. E. Bailey and D. R. Ollis, Biochemical Engineering Fundamentals, McGraw Hill, N.Y. (1977). The rate of growth of biomass is usually given by:
r_{X}=μC_{X}. (2)

The Jobses et al., (1985; 1986a) and Jobses (1986b) is a relatively simple unsegregated-structured model based on introducing an internal key compound (e) of the biomass. The activity of this compound is expressed in terms of concentrations of substrate, product, and the compound (e) of the biomass itself. So, the rate of formation of the key compound (e) is given by
rₑ=ƒ(C_{S})ƒ(C_{P})Cₑ, (3)
where the substrate dependence function ƒ(C_{S}) is given by the Monod-type relation,

$$\backslash f ( {C}_{S} ) = \frac{{C}_{S}}{{K}_{S} + {C}_{S}} . \qquad ( 4 )$$

The experimental data of Jobses et al. (1985; 1986a) and Jobses (1986b) showed that the relation between alcohol concentration C_{P} and alcohol dependence function ƒ(C_{P}) is a second order polynomial in C_{P} having the following form
ƒ(C_{P})=k₁−k₂C_{P}+k₃C_{P}². (5)

The model developed by Jobses et al. (1985; 1986a) and Jobses (1986b) is a four-dimensional model with the concentrations of substrate (S), product (P), microorganism or biomass (X) and the internal key component (e).

Based on this base model, we modified the dynamic model representing the concentrations of three components: X, S and P, together with a mass ratio of components e and X. We defined

$$E = \frac{{C}_{e}}{{C}_{X}} ≡ \frac{kg e}{kg X }$$

(thus, E is the fraction of biomass that is component (e)). The factor p used by Jobses et al. is the maximum possible specific growth rate (μₘₐₓ) that would be obtained if E=1, i.e., the whole biomass was active. We replace the factor p used by Jobses et al. by μₘₐₓ, thus, the specific growth rate can be written as

$$μ = \frac{{C}_{S} E {μ}_{max}}{{K}_{S} + {C}_{S}} ,$$

and the modified dynamic model is given by the following set of ordinary differential equations (6-9).

$$\begin{matrix}\frac{ⅆ E}{ⅆ t} = ( \frac{{k}_{1}}{{μ}_{max}} - \frac{{k}_{2}}{{μ}_{max}} {C}_{P} + \frac{{k}_{3}}{{μ}_{max}} {C}_{P}^{2} ) μ - μ E & ( 6 ) \\ \frac{ⅆ {C}_{X}}{ⅆ t} = μ {C}_{X} + D ( {C}_{XO} - {C}_{X} ) & ( 7 ) \\ \frac{ⅆ {C}_{S}}{ⅆ t} = - ( \frac{1}{{Y}_{SX}} μ + {m}_{S} ) {C}_{X} + D ( {C}_{SO} - {C}_{S} ) & ( 8 ) \\ \frac{ⅆ {C}_{P}}{ⅆ t} = ( \frac{1}{{Y}_{PX}} + {m}_{P} ) {C}_{X} + D ( {C}_{PO} - {C}_{P} ) & ( 9 )\end{matrix}$$

It is interesting to point out that the balance equation (6) for the mass ratio of component e and X (denoted by E) is independent of the type of reactor used. The equation states that the rate of formation of E (represented by

$$[ ( \frac{{k}_{1}}{{μ}_{max}} - \frac{{k}_{2}}{{μ}_{max}} {C}_{P} + \frac{{k}_{3}}{{μ}_{max}} {C}_{P}^{2} ) μ ] )$$

must be at least the same as the dilution rate of E (represented by μE). In equations (6) to (9), the value of μₘₐₓ is taken to be equal to 1 hr⁻¹ (Jobses et al., 1986a). If needed, equation (6) can be replaced by a differential equation for component e concentration:

$$\frac{ⅆ {C}_{e}}{ⅆ t} = ( {k}_{1} - {k}_{2} {C}_{P} + {k}_{3} {C}_{P}^{2} ) ( \frac{{C}_{S} {C}_{e}}{{K}_{S} + {C}_{S}} ) + D ( {C}_{eo} - {C}_{e} )$$

to get the same results.

It should also be noted that

$$D = \frac{q}{V}$$

(dilution rate), where q is the constant flow rate into the fermentor and V is the active volume of the fermentor, and both were taken as constant in the present modeling.

For steady state solutions, the set of four differential equations (6-9) reduces to a set of four coupled non-linear algebraic equations which can only be solved simultaneously.

Jobses et al. (1985; 1986a, 1986b) used the above four-dimensional model to successfully simulate the oscillatory behavior of an experimental continuous fermentor (without ethanol removal) in the high feed sugar concentration region.

By contrast, in the present modeling, the above-described modified Jobses et al. model was used to explore the different possible complex static/dynamic bifurcation behavior of this system in the two-dimensional (D−C_{SO}) parameter space and to study the implications of these phenomena on substrate conversion and ethanol yield and productivity.

Presentation Techniques and Numerical Tools Used

The bifurcation diagrams were obtained using the software package AUTO97. Doedel, E. J., Champneys, A. R., Fairgrieve, T. F., Kuznetsov, Y. A., Sandstede, B., and Wang, X. J. (1997). AUTO97; Continuation and bifurcation software for ordinary differential equations. Department of Computer Science, Concordia University, Montreal, Canada. This package is able to perform both steady-state and dynamic bifurcation analysis, including the determination of entire periodic solution branches using the efficient continuation techniques. Kubaiecek, M. and Marek, M. (1983). Computational methods in bifurcation theory and dissipative structures, Springer Verlag, N.Y.

The DIVPAG subroutine available with IMSL Libraries for FORTRAN (with automatic step size to ensure accuracy for stiff differential equations) was used for numerical simulation of periodic as well as chaotic attractors. A FORTRAN program was written for plotting the Poincaré plots.

The classical time trace and phase plane for the dynamics were used. However, for high periodicity and chaotic attractors these techniques are not sufficient. Therefore, other presentation techniques were used. These techniques are based upon the plotting of discrete points of intersection (return points) between the trajectories and a hypersurface (Poincaré surface) chosen at a constant value of the state variable (C_{X}=1.55 kg/m³, in the present modeling). These discrete points of intersection are taken such that the trajectories intersect the hyperplane transversally and cross it in the same direction.

The return points were used to construct the following important diagrams:

1. Poincaré one parameter bifurcation diagram: A plot of one of the co-ordinates of the return points (e.g., C_{S}) versus a bifurcation parameter (e.g., D).

2. Return point histogram: A plot of one of the co-ordinates of the return points (e.g., C_{S}) versus time.

Two rounds of modeling were performed before experimental verification. The first round does not include ethanol removal. The second round includes results for ethanol removal.

First Round Modeling

A 4-dimensional model for the anaerobic fermentation process to simulate the oscillatory behavior of an experimental continuous stirred tank fermentor was utilized to explore the static/dynamic bifurcation and chaotic behavior of a fermentor, which was shown to be quite rich. The modeling was a prelude to the second round of modeling and to the experimental exploration of bifurcation and chaos in a membrane fermentor.

Dynamic bifurcation (periodic attractors), as well as period doubling sequences leading to different types of periodic and chaotic attractors, were uncovered. It was fundamentally and practically important to discover the fact that in some cases, periodic and chaotic attractors have higher ethanol yield and production rate than the corresponding steady states.

The present investigation showed the rich static/dynamic bifurcation behavior of an example ethanol fermentation system. It also showed that the oscillations can be complex, leading to chaotic behavior, and that these periodic and chaotic attractors can be useful. Using the above model, it was shown that the average conversion of sugar and average yield/productivity of ethanol is sometimes higher for periodic and chaotic attractors than for the corresponding steady states, despite the fact that during oscillations the values of the state variables fall below the average value of the oscillations for some time. Borzani, W. (2001). “Variation of ethanol yield during oscillatory concentrations changes in undisturbed continuous ethanol fermentation of sugar-can blackstrap mollases,” World J. of Micro. and Biotech., 17(3), 253-258.

The model was used to explore the different possible complex static/dynamic bifurcation behavior of the fermentation system in the two-dimensional (D−C_{SO}) parameter space and to study the implications of these phenomena on substrate conversion and ethanol yield and productivity.

The system parameters for one of the experimental runs of Jobses et al. (1986a; 1986b) showing oscillatory behavior were used as the base set of parameters in the present modeling and are given in Table 1.

TABLE 1
The base set of parameters used.

| Parameter | Value |
|---|---|
| k₁ (hr⁻¹) | 16.0 |
| k₂ (m³/kg · hr) | 4.97 × 10⁻¹ |
| k₃ (m⁶/kg² · hr) | 3.83 × 10⁻³ |
| m_{S} (kg/kg · hr) | 2.16 |
| m_{P} (kg/kg · hr) | 1.1 |
| Y_{SX} (kg/kg) | 2.44498 × 10⁻² |
| Y_{PX} (kg/kg) | 5.26315 × 10⁻² |
| K_{S} (kg/m³) | 0.5 |
| C_{XO} (kg/m³) | 0 |
| C_{PO} (kg/m³) | 0 |
| C_{eO} (kg/m³) | 0 |

Modeling Results and Discussion

The results of the bifurcation analysis are classified below in two different sections:
A) dilution rate (D) as the bifurcation parameter and
B) feed sugar concentration (C_{SO}) as the bifurcation parameter.
   The reason for choosing these two bifurcation parameters (D and C_{SO}) is that they are the easiest to manipulate during the operation of a laboratory or full-scale fermentor.

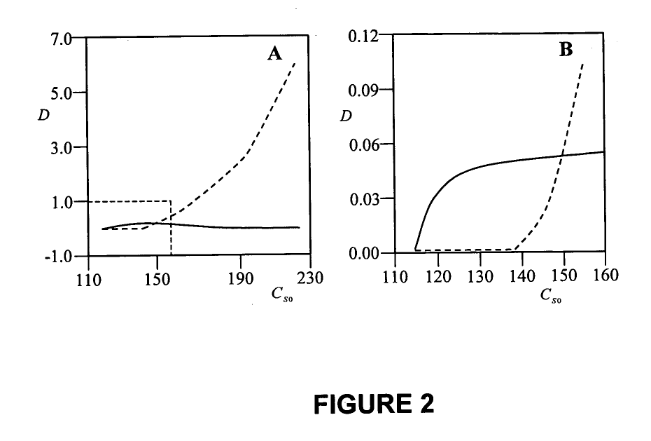

FIG. 2A is a two-parameter continuation diagram of D vs. C_{SO} showing the loci of static limit points (SLPs) and HB points. One-parameter bifurcation diagrams are constructed by 1) taking a fixed value of C_{SO} and constructing the D bifurcation diagrams then 2) taking fixed values of D and constructing the C_{SO} bifurcation diagrams. FIG. 2B is an enlargement of dotted box of FIG. 2A.

In order to evaluate the performance of the example fermentor as an alcohol producer, we calculated the conversion of substrate, the product (ethanol) yield, and its productivity (performance measurement parameters) according to the simple relations incorporated into the FORTRAN programs,

$$\begin{matrix}Substrate ( sugar ) conversion = {X}_{S} = \frac{{C}_{SO} - {C}_{S}}{{C}_{SO}} \\ Ethanol yield = {Y}_{P} = \frac{{C}_{P} - {C}_{PO}}{{C}_{SO}}\end{matrix}$$

Ethanol productivity (production rate per unit volume, kg/m³·hr)=P_{P}=C_{P} D

For the oscillatory and chaotic cases also, the average conversion \overline{X}_{S}, average yield \overline{Y}_{P}, and the average production rate \overline{P}_{P}, as well as the average ethanol concentration \overline{C}_{P} were computed. They are defined as

$${\overline{X}}_{S} = {\int }_{0}^{τ} {X}_{S} \frac{ⅆ t}{τ} , {\overline{Y}}_{P} = {\int }_{0}^{τ} {Y}_{P} \frac{ⅆ t}{τ} , {\overline{P}}_{P} = {\int }_{0}^{τ} {P}_{P} \frac{ⅆ t}{τ} , {\overline{C}}_{P} = {\int }_{0}^{τ} {C}_{P} \frac{ⅆ t}{ⅆ τ} ,$$

where the τ values in the periodic cases represent one period of the oscillations, and in the chaotic cases, they are taken to be long enough to be a reasonable representation of the “average” behavior of the chaotic attractor.
A) Dilution Rate D as the Bifurcation Parameter
Case (A-1): C_{SO}=140 kg/m³

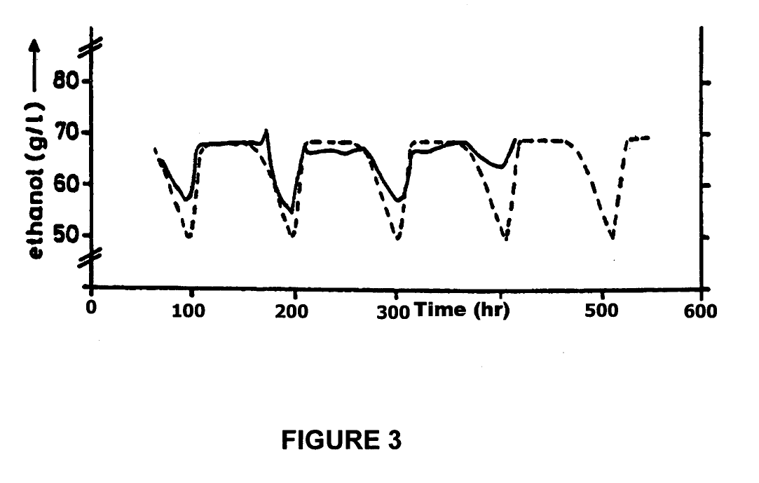

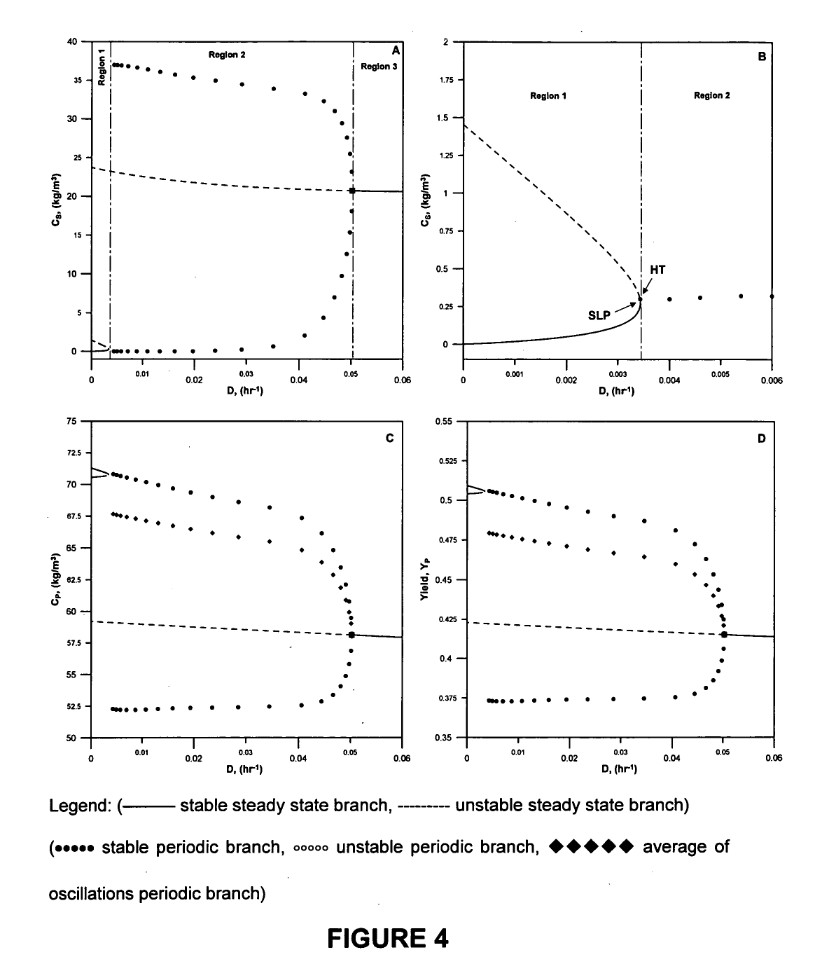

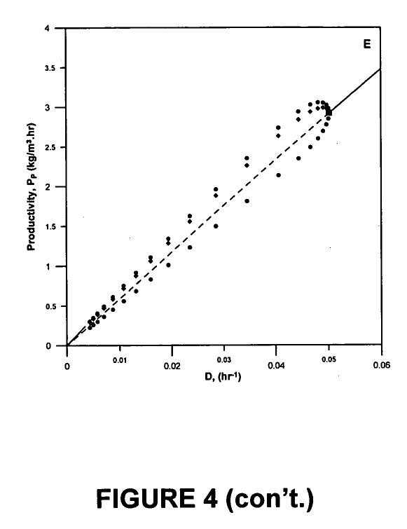

Jobses et al. (1986a; 1986b) used this value of C_{SO} in their experiments together with a dilution rate of D=0.022 hr⁻¹. An example of the comparison between the dynamic modeling and experimental results obtained by Jobses et al. (1986a) is shown in FIG. 3. Details of static and dynamic bifurcation behavior for this case are shown in FIGS. 4A-E, with the dilution rate D as the bifurcation parameter.

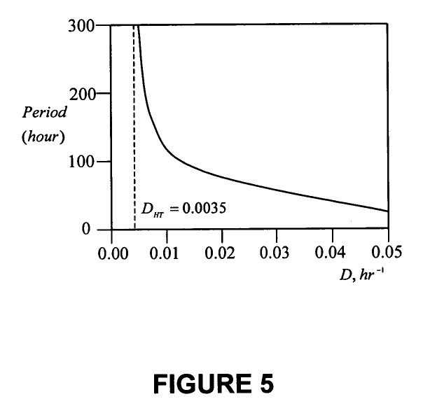

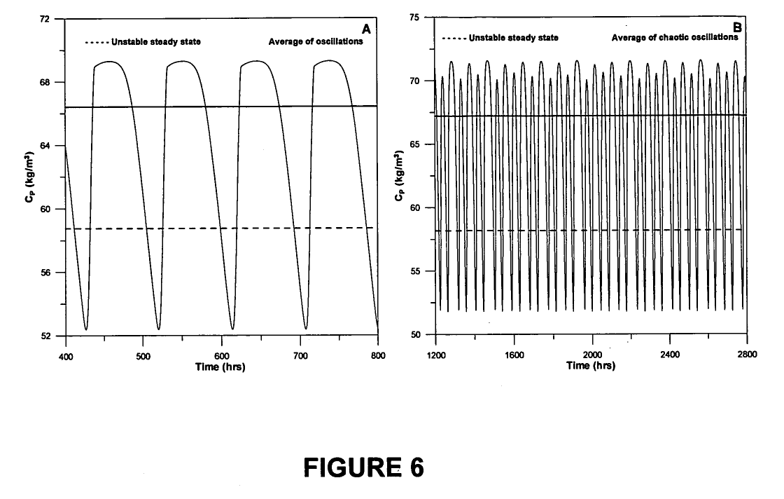

FIG. 4A shows the bifurcation diagram for substrate concentration (C_{S}) with clear demarcations between the different regions using dotted vertical lines. The bifurcation diagram has 3 regions. It is clear that the static bifurcation diagram is an incomplete S-shape hysteresis-ype with a static limit point (SLP) at very low value of D=0.0035 hr⁻¹. The dynamic bifurcation shows a Hopf bifurcation (HB) at D_{HB}=0.05 hr⁻¹ with a periodic branch emanating from it. The region in the neighborhood of the SLP is enlarged in FIG. 4B. The periodic branch emanating from HB terminates homoclinically (with infinite period) when it touches the saddle point very close to the SLP at D_{HT}=0.0035 hr⁻¹. FIG. 4C is the bifurcation diagram for the ethanol concentration (C_{P}). It is clear from FIG. 4C that the average ethanol concentrations for the periodic attractors are higher than those corresponding to the unstable steady states. FIGS. 4D and 4E show the bifurcation diagrams for ethanol yield (Y_{P}) and ethanol production rate (P_{P}), where the average yield (\overline{Y}_{P}) and production rate (\overline{P}_{P}) for the periodic branch are shown as diamond-shaped points. FIG. 5 shows the period of oscillations as the periodic branch approaches the homoclinical bifurcation point; the period tends to infinity indicating homoclinical termination of the periodic attractor at D_{HT}=0.0035 hr⁻¹. Keener, J. P. (1981). “Infinite period bifurcation and global bifurcation branches,” J. Appl. Math., 41, 127-144.

1. Region 1: This region has three point attractors (D<D_{HT}) and is characterized by the fact that two of them are unstable and only the steady state with the highest conversion is stable. The highest conversion (almost complete conversion) occurs in this region for the upper stable steady state. This steady state also gives the highest ethanol yield which is equal to 0.51 (FIG. 4D). On the other hand, this region has the lowest ethanol production rate (FIG. 4E) due to the low values of the dilution rate D (for a given fermentor active volume, it corresponds to very low flow rate).

2. Region 2: The region of D_{HB}>D>D_{HT} is characterized by a unique periodic attractor (surrounding the unstable steady state) which starts at the HB point and terminates homoclinically at a point very close to SLP as shown in FIGS. 4A and 4B.

It is clear that in this region, the average of the oscillations for the periodic attractor gives (as shown in FIGS. 4C-E) higher \overline{C}_{P}, \overline{Y}_{P}; and \overline{P}_{P} than that of the corresponding steady states, which means that the operation of the fermentor under periodic conditions is not only more productive but will also give higher ethanol concentrations by achieving higher sugar conversion. Comparison between the values of the static branch and the average of the periodic branch in this region (e.g., at D=0.045 hr⁻¹) shows that the percentage improvements are

| parameter | percentage improvement |
|---|---|
| \overline{C}_{P} | 9.34% |
| \overline{X}_{S} | 9.66% |
| \overline{Y}_{P} | 8.67% |
| \overline{P}_{P} | 9.84% |

Therefore, the best production policy for ethanol concentration, yield, and productivity for this case is a periodic attractor. In general, there is a trade-off between concentration and productivity, which requires economic optimization study to determine the optimum D.

The phenomenon of possible increase of conversion, yield, and productivity through deliberate unsteady state operation has been known for some time (Douglas, J. M. (1972). Process Dynamics and Control, Volume 2, Control System Synthesis. Prentice Hall, N.J., USA). Deliberate unsteady operation is associated with non-autonomous (externally forced) systems. In the present work, the unsteady state operation of the system (periodic operation) is an intrinsic characteristic of the system in certain regions of the parameters. Moreover, this system intrinsically shows not only periodic attractors but also chaotic attractors.

Static and dynamic bifurcation and chaotic behavior are due to the non-linear coupling of the system (Elnashaie and Elshishini, 1996). This non-linear coupling is the cause of all the phenomena including the possibility of higher conversion, yield, and productivity. Physically it is associated with the unequal excursion of the dynamic trajectory (periodic or chaotic) above and below the unstable steady state (FIG. 6). It is clear from FIG. 6 that the excursion above the unstable steady state (for both the periodic attractor in FIG. 6A and the chaotic attractor in FIG. 6B) is not only much higher than its excursion below it, but it is also for a longer time.

It is fundamentally and practically important to notice that conversion, yield, and productivity are very sensitive to D changes in the neighborhood of the HB point. This sensitivity is not only qualitative regarding the birth of oscillations for D<D_{HB}, but also quantitative comparing the conversion, yield, and productivity for D>D_{HB} and their average values for D<D_{HB}. The further decrease in D beyond D_{HB} causes the average values of conversion, yield, and productivity to increase, but not as sharp as in the neighborhood of D_{HB}.

3. Region 3: This region is characterized by the existence of a unique stable steady state having the conversion, yield, and productivity characteristics very close to those of the unstable steady state in Region 2.
Case (A-2): C_{SO}=149 kg/m³

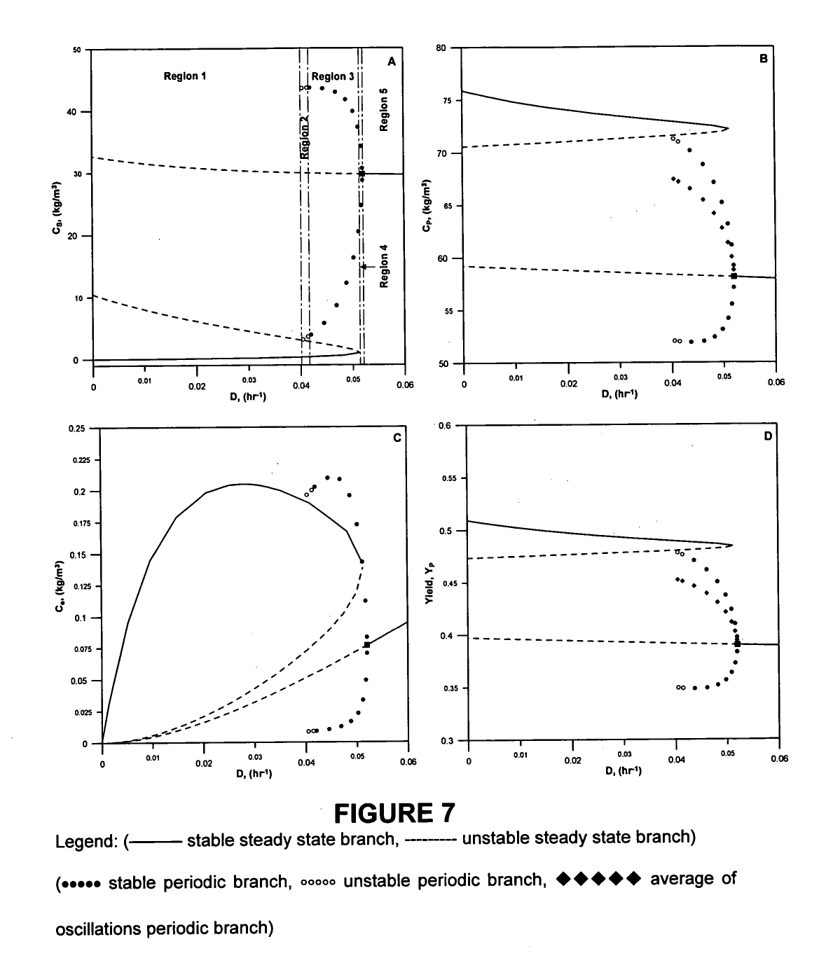

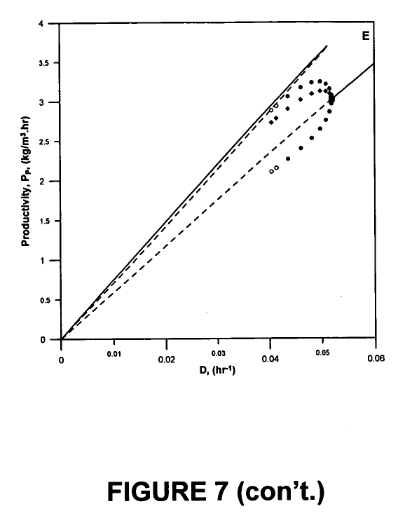

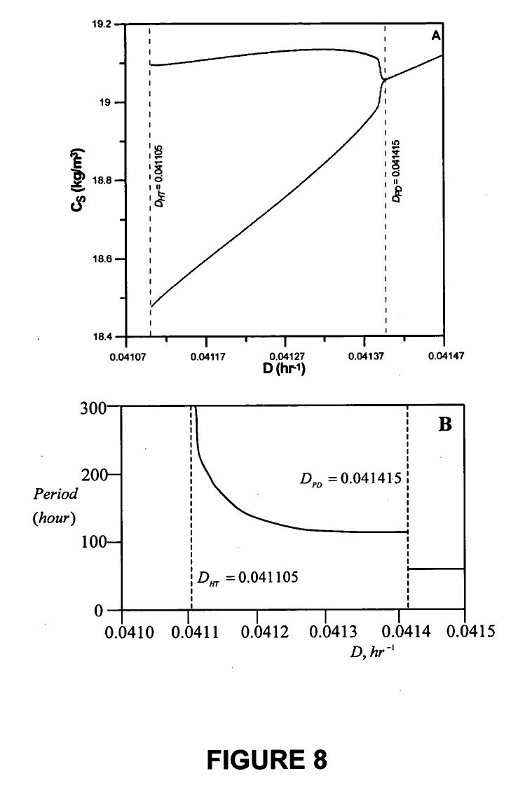

FIGS. 7A-7E show the bifurcation diagrams for this case with D as the bifurcation parameter. The bifurcation diagram is again an incomplete S-shaped hysteresis-type with the static limit point (SLP) shifted to much higher value of D_{SLP}=0.051 hr⁻¹ compared with the previous case. A unique periodic attractor exists between the Hopf bifurcation point at D_{HB}=0.0515 hr⁻¹ and D_{SLP} followed by a region of bistability characterized by stable periodic and point attractors between D_{SLP} and the first period doubling point at D_{PD}=0.041415 hr⁻¹. In this region, each of the two attractors will have its domain of attraction. This, of course, will have its important practical implications not only with regard to start-up, but also control policies. The amplitudes of the oscillations increase as D decreases, as shown in FIG. 7A. However, in this case, in contradiction to the previous case (A-1), prior to the HT a complex period doubling (PD) scenario starts. Period doubling (PD) occurs at D_{PD}, as shown by the Poincaré diagram in FIG. 8A and the period vs. D diagram in FIG. 8B. At this point the periodicity of the system changes from period one (P1) to period two (P2). FIG. 8A shows that as D decreases further, the periodic attractor (P2) grows in size until it touches the middle unstable saddle type steady state and the oscillations disappear homoclinically at D_{HT}=0.041105 hr⁻¹ without completing its Feigenbaum period doubling sequence to chaos (Feigenbaum. M. J. (1980). “Universal behaviour in nonlinear systems,” Los Alamos Sci., 1, 4-36).

The bifurcation diagram has 5 regions:

1. Region 1: Region 1, where D<D_{HT}, is characterized by the existence of three steady states. Two of these are unstable and only one steady state with very high conversion (the lowest branch in FIG. 7A which is the topmost branch in FIG. 7B) is stable. Ce shows a non-monotonic behavior. It initially increases as D decreases until it reaches a maximum value of 0.2 kg/m³ at D=0.025 hr⁻¹, then it decreases continuously towards zero. This non-monotonic behavior is due to the non-linear term ƒ(C_{P})=k₁−k₂ C_{P}+k₃ C_{P}², as in this region this function shows a non-monotonic behavior for the corresponding values of C_{P}.

2. Region 2: This region of D_{PD}>D>D_{HT} is characterized by bistability with its associated start-up and control considerations. There is a very high conversion stable static branch together with a stable period two (P2) branch.

3. Region 3: This region of D_{SLP}>D>D_{PD} is also characterized by bistability. There is a very high conversion stable static branch as well as a stable periodic branch. It is again clear that the average of \overline{X}_{S}, \overline{Y}_{P}, and \overline{P}_{P} values for the periodic branch are higher than the corresponding unstable steady states. Comparison between the values of the static branch and the average of the periodic branch at D=0.045 hr⁻¹ shows that the percentage improvements are

| parameter | percentage improvement |
|---|---|
| \overline{C}_{P} | 13.02% |
| \overline{X}_{S} | 13.33% |
| \overline{Y}_{P} | 13.02% |
| \overline{P}_{P} | 13.577% |

(FIGS. 7B, 7D and 7E).
4. Region 4: The very narrow Region 4 of D_{HB}>D>D_{SLP} has a unique periodic attractor with period one (P1) which emanates from the HB point at which the stable static branch looses its stability and becomes unstable as D decreases (FIG. 7A).
5. Region 5: Region 5 of D>D_{HB} has a unique stable static attractor (FIG. 7A).
Case (A-3): C_{SO}=150.3 kg/m³

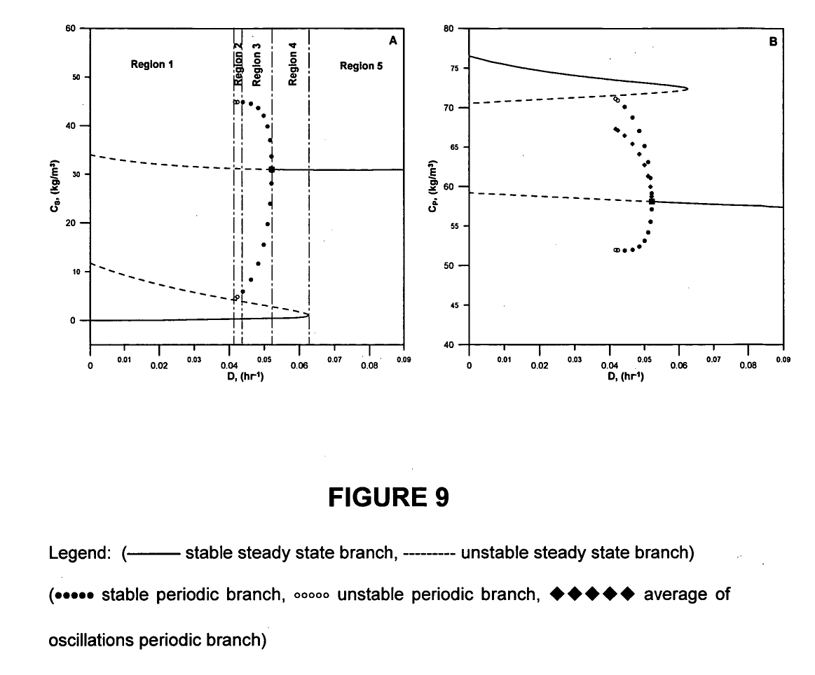

FIGS. 9A and 9B show the static and dynamic bifurcation diagrams with the dilution rate D as the bifurcation parameter for this higher sugar feed concentration. The bifurcation diagram has 3 regions. The bifurcation diagram is again an incomplete S-shaped hysteresis-type with a static limit point (SLP) at D_{SLP}=0.062 hr⁻¹. The dynamic bifurcation shows a Hopf bifurcation (HB) with a periodic branch emanating from it at D_{HB}=0.052 hr⁻¹, where the amplitudes of the oscillations increase as D decreases.

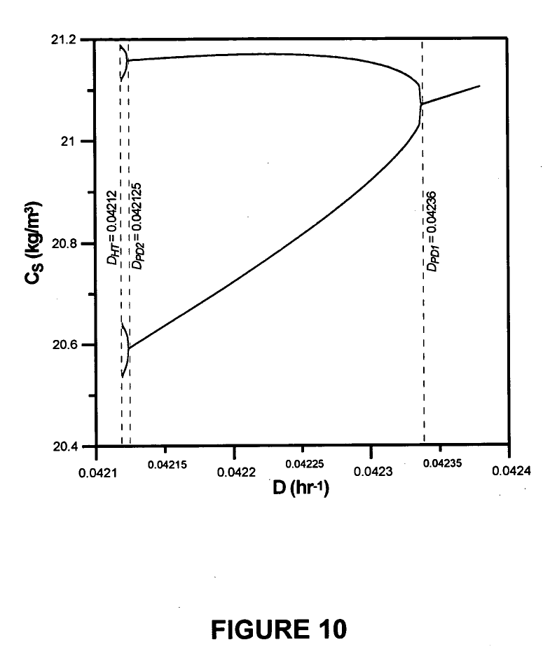

The Poincare bifurcation diagram (FIG. 10) shows that the periodicity of the system changes from period one (P1) to period two (P2) to period four (P4) in a sequence of incomplete Feigenbaum period doubling to chaos. The first (PD1) point is at D=0.04236 hr⁻¹, and the second point (PD2) at D=0.042125 hr⁻¹; the periodic attractor terminates homoclinically with periodicity four (P4) at D=0.04212 hr⁻¹ with infinite period.

1. Region 2: Region 2 of D_{PD1}>D>D_{HT} is characterized by bistability where there is a very high conversion stable static branch as well as stable periodic branches of different periodicities (P2, P4). This region is the characteristic region of this case due to the presence of an incomplete period doubling sequence of P2 to P4, as shown in FIG. 10.

2. Region 3: Region 3 of D_{HB}>D>D_{PD1} is also characterized by bistability where there is a very high conversion stable static branch as well as a stable periodic branch with period one (P1). Comparison between the values of average of a periodic attractor and the corresponding unstable steady state at D=0.045 hr⁻¹ shows an improvement with the following percentages:

| parameter | percentage improvement |
|---|---|
| \overline{C}_{P} | 14.099% |
| \overline{X}_{S} | 13.776% |
| \overline{Y}_{P} | 14.099% |
| \overline{P}_{P} | 13.973% |

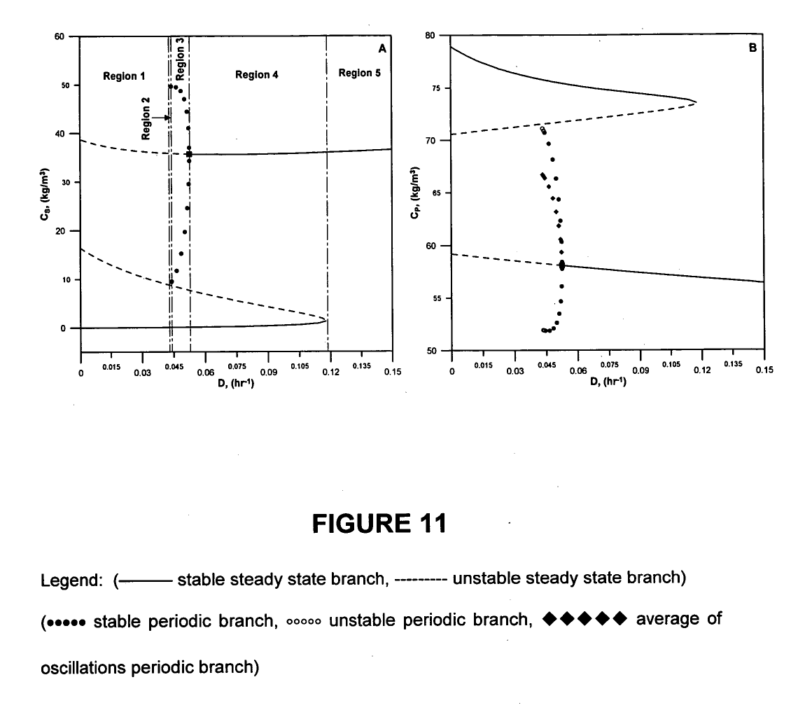

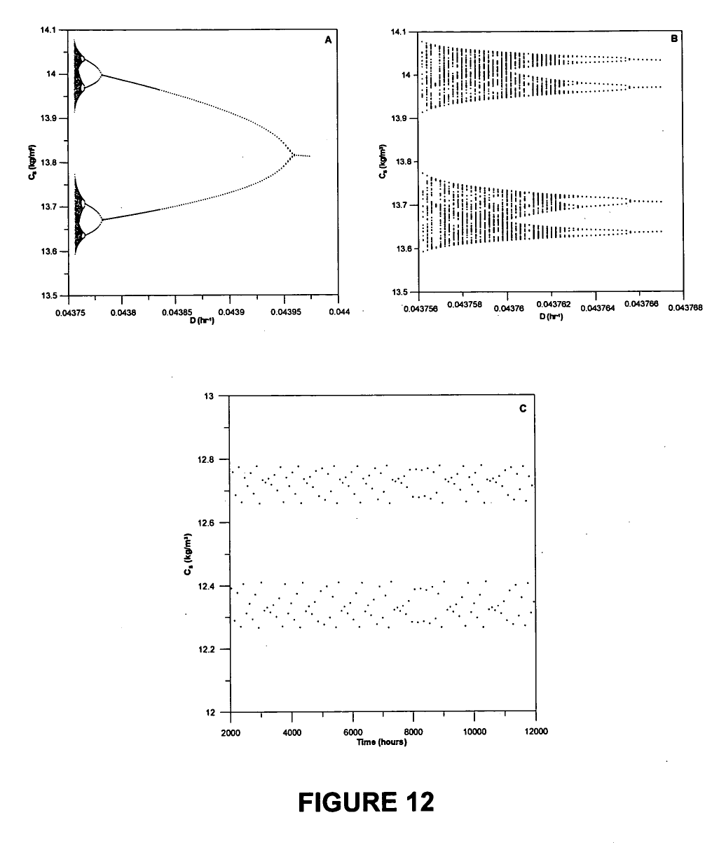

The behavior of this case (A-4) is qualitatively very similar to the previous case (A-3) as shown in FIGS. 11A and 11B. The bifurcation diagram has 5 regions. The main difference between this case and case (A-3) is that the period doubling sequence completes its Feigenbaum sequence to banded chaos as shown in FIG. 12. This particular case is characterized by the existence of banded chaos which terminates homoclinically.

1. Region 2: Region 2 of D_{PD1}>D>D_{HT} is, therefore, the characteristic region of this case due to the presence of period doubling to the banded chaos (two bands); the sequence being P1→P2→P4→P8→•••→ Banded Chaos, which terminates homoclinically at D_{HT}=0.043755 hr⁻¹, as shown in FIG. 12A. The chaotic region is enlarged in FIG. 12B where the two bands of chaos and period doubling sequence are clearly shown. FIG. 12C is the return point histogram for variable C_{S} at D=0.04376 hr⁻¹. The return points are taken where the trajectories cross a certain hypothetical plane (Poincaré surface, here, C_{X}=1.55 kg/m³) transversally and in the same direction. Comparison of the values of the average of chaotic oscillations and corresponding steady state at D=0.04385 hr⁻¹ shows the following percentage improvements:

| parameter | percentage improvement |
|---|---|
| \overline{C}_{P} | 14.471% |
| \overline{X}_{S} | 14.52% |
| \overline{Y}_{P} | 14.383% |
| \overline{P}_{P} | 16.561% |

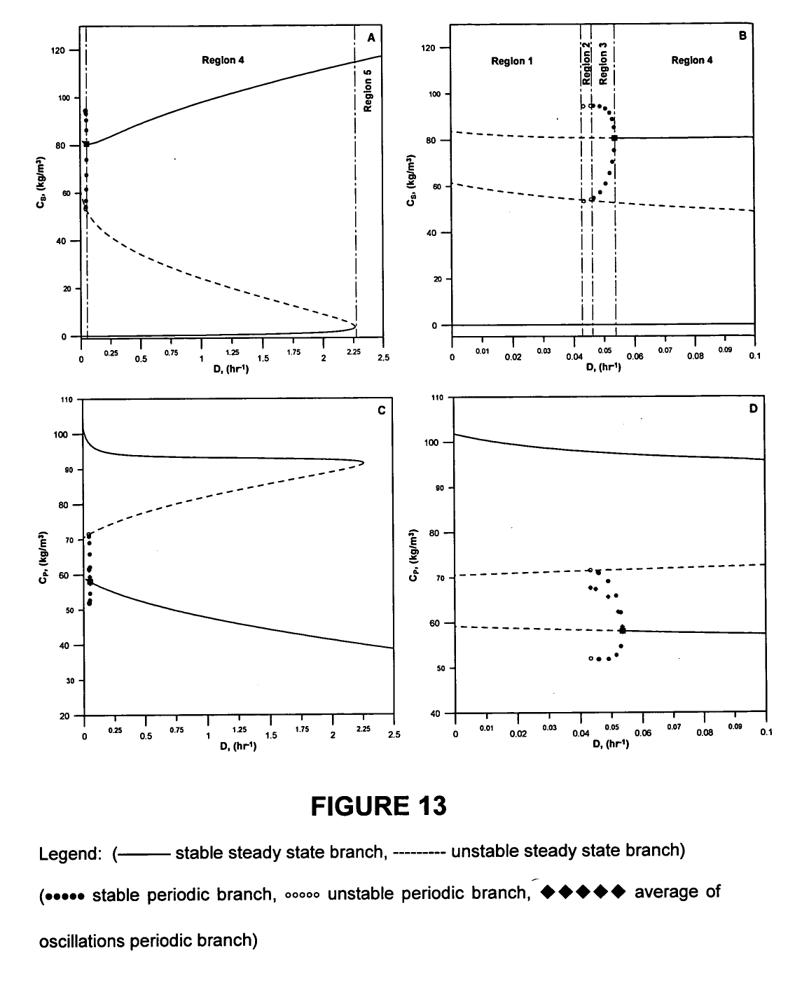

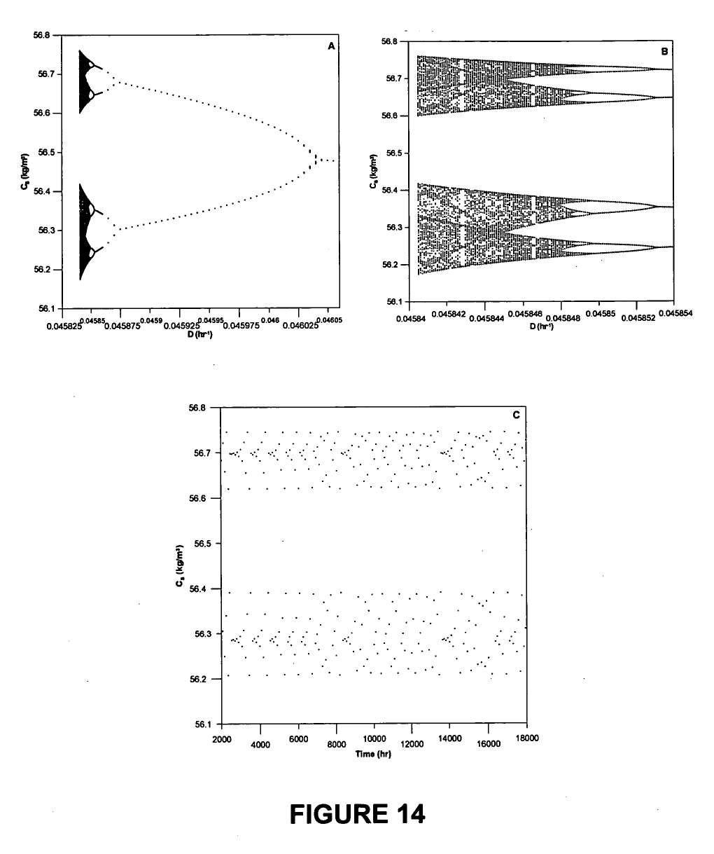

This is a case with a very high feed sugar concentration. FIGS. 13A-13D show the static and dynamic bifurcation diagrams with the dilution rate (D) as the bifurcation parameter, and the corresponding enlargement of the chaotic region. The bifurcation diagram has 5 regions. This case is characterized by the existence of fully developed chaos in Region 2. This region is the characteristic region of this case due to the presence of period doubling to the fully developed chaos (two bands); sequence is P1→P2→P4→P8→•••→ Fully Developed Chaos, which terminates homoclinically at D_{HT}=0.045835 hr⁻¹ (FIG. 13B). FIG. 14A (one-dimensional Poincaré diagram) is enlarged in FIG. 14B, where the two bands of chaos and the period doubling sequence are clearly shown. FIG. 13C is the return point histogram (with the Poincaré surface at C_{X}=1.55 kg/m³) for variable C_{S} at D=0.04584 hr⁻¹.

B) Feed Sugar Concentration (C_{SO}) as the Bifurcation Parameter
Case (B-1): Dilution rate D=0.05 hr⁻¹

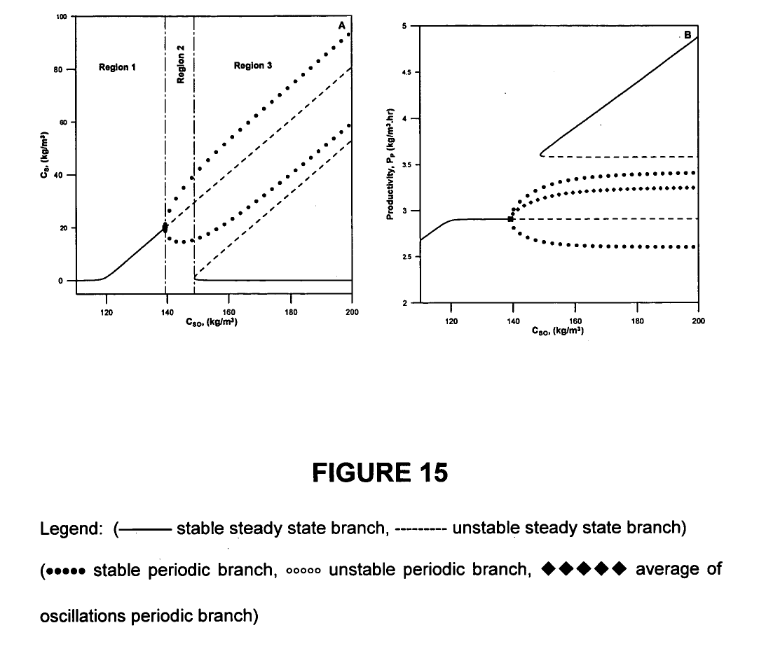

FIGS. 15A and 15B show the static and dynamic bifurcation diagram with the substrate feed concentration C_{SO} as the bifurcation parameter for a fixed value of the dilution rate (D=0.05 hr⁻¹). The bifurcation diagram has 3 regions. For this case, there is a static limit point (SLP) at a relatively high value of feed sugar concentration of C_{SO SLP}=148 kg/m³. The dynamic bifurcation shows a Hopf bifurcation point (HB) at C_{SO HB}=140 kg/m³ after which sustained stable oscillations are observed with increasing amplitudes as C_{SO} increases (the periodic attractor does not terminate homoclinically within the given physically realistic range of the bifurcation parameter C_{SO}).

1. Region 1: Region 1 of C_{SO}<C_{SO HB} has only one stable steady state (FIGS. 15A and 15B). In this region, as C_{SO} increases, the substrate concentration C_{S} slightly increases from 0.079 to 0.239 kg/m³ initially (in the range 110<C_{SO}<115.87, this is due to the fact that the sugar fed is consumed totally by the microorganisms). After this point, the value of sugar concentration increases steadily to 20.014 kg/m³ (FIG. 15A).

2. Region 2: Region 2 of C_{SO SLP}>C_{SO}>C_{SO HB} is characterized by a unique periodic attractor with period one. Again, like the previous cases, it is observed that the average values of the oscillations are higher than the corresponding unstable steady state values. Comparison of the values of average of the oscillations and corresponding steady state at C_{SO}=160 kg/m³ shows an improvement of the following percentages:

| parameter | percentage improvement |
|---|---|
| \overline{C}_{P} | 9.989% |
| \overline{X}_{S} | 10.281% |
| \overline{Y}_{P} | 9.982% |
| \overline{P}_{P} | 9.989% |

(FIG. 15B).
Case (B-2): Dilution rate D=0.045 hr⁻¹

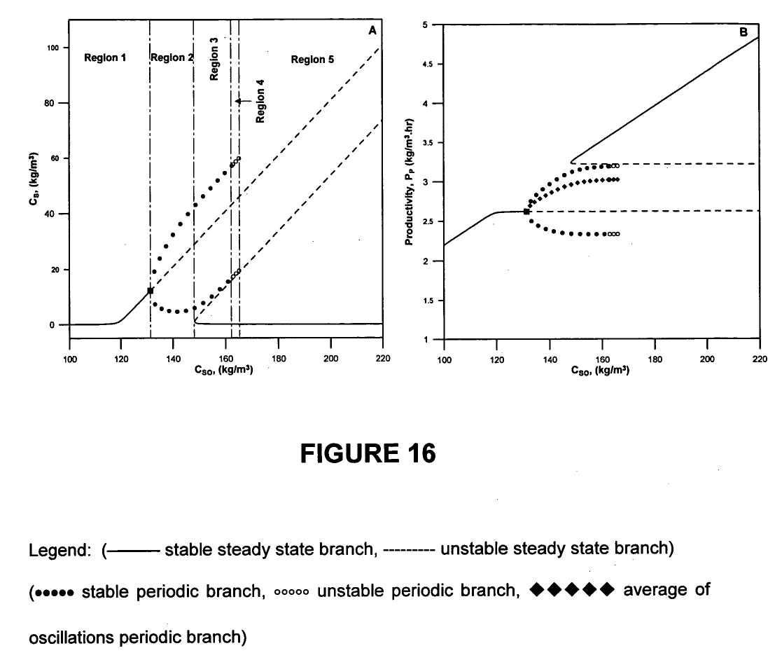

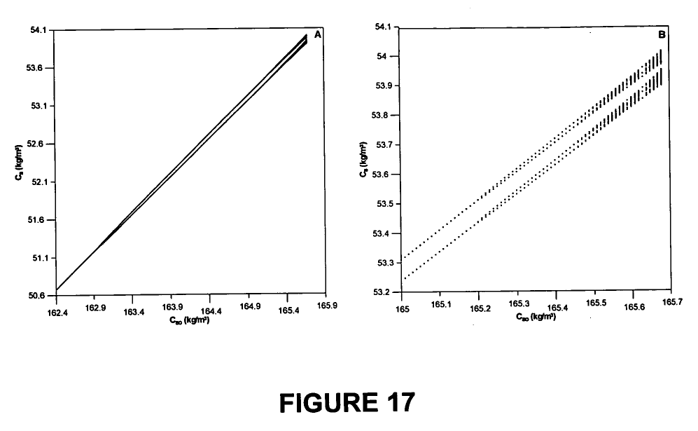

This case is characterized by the presence of period doubling route to banded chaos and subsequent homoclinical termination of this chaotic attractor as shown in FIGS. 16 and 17.

The bifurcation diagram has 5 regions.

Region 4 of C_{SO PD}<C_{SO}<C_{SO HT} (i.e., 163.07<C_{SO}<165.7) is the characteristic region of this case having bistability with a periodic/chaotic attractor and a stable high conversion static attractor (FIGS. 16A and 16B). The periodic branch in this region changes its periodicity in a period doubling sequence leading to chaos, and the chaotic attractor terminates homoclinically at C_{SO HT}=165.7 kg/m³ as shown in FIGS. 17A (FIG. 17B is the enlargement of the chaotic region of FIG. 17A). Comparison of the values of average of the periodic oscillations and corresponding steady state at C_{SO}=160 kg/m³ shows the following percentage improvements:

| parameter | percentage improvement |
|---|---|
| \overline{C}_{P} | 15.064% |
| \overline{X}_{S} | 15.376% |
| \overline{Y}_{P} | 15.15% |
| \overline{P}_{P} | 15.064% |

(FIG. 16B).
Conclusions and Recommendations

The model for the anaerobic fermentation process developed and used by Jobses et aL. (1985; 1986a; 1986b) to simulate the oscillatory behavior of an experimental fermentor was utilized in this preliminary modeling of the non-linear dynamics of the system to study the steady state as well as the dynamic oscillations in an experimental fermentor with Zymomonas mobilis at the high sugar concentration range. This non-linear dynamics investigation was a prelude to a second round modeling and an experimental study to verify the findings.

The present modeling revealed the rich static and dynamic bifurcation behavior of this four-dimensional system, which includes bistability, incomplete period doubling cascade, period doubling to banded chaos, and homoclinical (infinite period) bifurcation for periodic as well as chaotic attractors. The investigation concentrated on the effect of the different values of the dilution rate and substrate feed concentration on the bifurcation/chaotic behavior of the system. Special emphasis was given to the implication of these phenomena on the sugar conversion, ethanol yield, and productivity of the fermentation process.

It is well known from the dynamical system theory that these experimentally observed (and mathematically simulated) oscillations must start and end at certain critical points. Therefore, the model was used to investigate the rich static and dynamic bifurcation behavior of this example experimental fermentor over a wide range of parameters. The bifurcation parameters chosen in this investigation were the dilution rate (D) and the feed sugar concentration (C_{SO}). This was not only because of their importance, but also because they are the easiest to manipulate in an experimental or industrial setup.

Two parameter continuation diagrams (TPCD) were constructed for the loci of static limit points (SLP) and Hopf bifurcation (HB) points with D and C_{SO} as the two parameters. Vertical and horizontal sections were taken on the TPCD at chosen values of D and C_{SO}, and one-parameter bifurcation diagrams were constructed for all system variables as well as conversion, ethanol concentration, and ethanol production rate.

At relatively low substrate concentration of feed, the periodicity of the periodic attractor is P1 which (at some value of bifurcation parameter) terminates homoclinically by touching the saddle. Increasing the substrate concentration of the feed makes the oscillatory behavior of the system double its periodicity once to period two, or twice to period four, or three times to period eight, depending upon the feed concentration. Further increase in C_{SO} gives small-banded chaos, leading ultimately to two fully developed bands of chaos.

In all of these cases, the periodic branch emanated from a Hopf bifurcation point and terminated homoclinically. Table 2 shows the location of the Hopf bifurcation points, the homoclinical termination point, the static limit point, and the type of the periodic attractor before the homoclinical termination with respect to the dilution rate in five different cases.

TABLE 2
Conclusion table for different cases investigated.
With dilution rate (D) as bifurcation parameter

| | | | | Type of periodic |
|---|---|---|---|---|
| | | | | attractor before |
| C_{SO} | D_{HB} | D_{SLP} | D_{HT} | homoclinical |
| (kg/m³) | (hr⁻¹) | (hr⁻¹) | (hr⁻¹) | termination (HT) |
| 140 | 5.00 × 10⁻² | 3.60 × 10⁻³ | 3.50 × 10⁻³ | Period I |
| 149 | 5.15 × 10⁻² | 5.10 × 10⁻² | 4.11 × 10⁻² | Period II |
| 150.3 | 5.20 × 10⁻² | 6.20 × 10⁻² | 4.21 × 10⁻² | Period IV |
| 155 | 5.30 × 10⁻² | 1.18 × 10⁻¹ | 4.3755 × 10⁻² | Banded Chaos |
| 200 | 5.40 × 10⁻² | 2.25 | 4.5835 × 10⁻² | Fully Developed Chaos |

With feed sugar concentration (C_{SO}) as bifurcation parameter

| D | C_{SO HB} | C_{SO SLP} | C_{SO HT} | |
|---|---|---|---|---|
| (hr⁻¹) | (kg/m³) | (kg/m³) | (kg/m³) | |
| 0.05 | 140.0 | 148.0 | — | No HT |
| 0.045 | 132.0 | 147.0 | 165.7 | Banded Chaos |

As shown in Table 2, when C_{SO} increases (with D as the bifurcation parameter), the positions of HB and SLP move to the right (increasing D), but the speed of movement of SLP is greater than that of HB. This prevents the formation of ordinary fully developed chaos because the distance between the HB point (where the periodic branch emanates from) and the saddle (where the periodic branch terminates at) is not sufficient to produce fully developed chaos. The same observation is true when we take C_{SO} to be the bifurcation parameter.

In the ranges which include periodic and chaotic attractors, the operation of the reactor under these periodic/chaotic conditions give higher average sugar conversion, ethanol yield, and productivity than those of the corresponding unstable steady states.

The results are fundamentally and practically important. They can be summarized in the following points:

1. The system showed static bifurcation (multiplicity of the steady state) over a wide range of parameters.

2. In the simplest cases, a HB point existed on one of the static branches and the periodic branch emanating from it terminated homoclinically at an infinite period bifurcation (HT point) when the periodic attractor touched the saddle type steady state in the multiplicity region.

3. In more complex cases, the periodic branch showed an incomplete period doubling sequence which did not develop into chaos because the higher periodicity attractors touched the saddle type steady state and terminated homoclinically before it had completed the well-known Feigenbaum period doubling sequence to chaos.

4. In other more complex cases, the period doubling sequence completed its route to chaos, giving a region of chaotic behavior.

5. Analysis of the periodic and chaotic regions showed that in these regions the average sugar conversion, ethanol yield, and production rate of the periodic and chaotic attractors can be higher than for corresponding unstable steady state values.

The extension of this four-dimensional model to a higher dimensional model incorporating continuous ethanol removal (membrane fermentors) to overcome the product inhibition is discussed next in the SECOND ROUND MODELING section. The experimental results below confirm the validity of the model for the accurate description of the chaotic behavior, as was confirmed by Jobses et al. for static and periodic operation.

Second Round Modeling

We used the model discussed above to explore the behavior of an example fermentor system for a wide range of physically realistic parameters. We showed the rich static/dynamic bifurcation behavior of this system. The extensive quantitative and qualitative analysis of the fermentor confirmed the presence of bifurcation/chaotic phenomena over a wide range of parameters. The analysis also showed that these oscillations can be complex leading to chaotic behavior and that these periodic and chaotic attractors can be useful. It was shown that operating the system at periodic and chaotic states gives higher ethanol productivity/yield and sugar conversion as compared to the operation at the corresponding steady state. It was shown that the average conversion of sugar and average yield/ productivity of ethanol is sometimes higher for periodic and chaotic attractors than for the corresponding steady states despite of the fact that during oscillations, the values of the state variables fall below the average value of the oscillations for some time. W. Borzani, “Variation of ethanol yield during oscillatory concentrations changes in undisturbed continuous ethanol fermentation of sugar-can blackstrap mollases,” World J. Microbiol. Biotechnol. 17(3), 253-258 (2001).

Furthermore, the effect of introducing an ethanol selective membrane was investigated and new phenomena discovered. It was shown that the ethanol removal membrane acts as a stabilizing controller for the fermentor.

The mathematical model used predicted the experimental oscillations and other many complicated phenomena in certain regions of the parameters. More importantly from the non-linear dynamics point of view, these simple oscillations bifurcate into more complex phenomena like chaos with change in the values of some parameters.

Integrating these phenomena of non-linear dynamics with membrane science (i.e., using a permselective membrane to remove product ethanol) gave even higher yield and productivity of ethanol and also stabilized the fermentor (thus, acting as a controller to eliminate instabilities).

In-situ Ethanol Removal

Since ethanol produced is an inhibitor for the microorganisms used as biocatalysts, it is important for efficient production to use a suitable technique for continuous removal of product ethanol. Continuous ethanol removal from fermentation broths has been accomplished by vacuum distillation (B. L. Maiorella, H. W. Blanch and C. R. Wilke, Lawrence Berkeley Lab., Berkeley, Calif., USA. “Vacuum ethanol distillation technology,” Energy Res. Abst. Abstr. No.29317, 8(12), 166 pp (1983); J. Sundquist, H. W. Blanch and C. R. Wilke, “Vacuum fermentation,” Bioprocess. Technol. 11(Extr. Bioconversion), 237-258 (1991)), solvent extraction (M. Minier and G. Goma, “Production of ethanol by coupling fermentation and solvent extraction,” Biotechnol. Let. 3(8), 405-408 (1981); F. Kollerup and A. J. Daugulis, “Ethanol production by extractive fermentation-solvent identification and prototype development,” Can. J. Chem. Eng. 64(4),598-606 (1986); M. T. B. Nomura and S. Nakao, “Selective Ethanol Extraction from Fermentation Broth using a Silicate Membrane,” Sep. Purif. Technol. 27, 59-66 (2002)), and membrane pervaporation (Y. Mori and T. Inaba, “Ethanol production from starch in a pervaporation membrane bioreactor using Clostridium thermohydrosulfuricum,” Biotechnol. Bioeng. 36(8), 849-853 (1990); Y. Shabtai, S. Chaimovitz, A. Freeman, E. Katchalski-Katzir, C. Linder, M. Nemas, M. Perry and O. Kedem, “Continuous ethanol production by immobilized yeast reactor coupled with membrane pervaporation unit,” Biotechnol. Bioeng. 38(8), 869-876 (1991); W. J. Groot, M. R. Kraayenbrink, R. H. Waldram, R. G. J. M. van der Lans and C. A. M. Luyben, “Ethanol production in an integrated process of fermentation and ethanol recovery by pervaporation,” Bioprocess Eng. 8(3-4), 99-111 (1992); Y. Shabtai and C. Mandel, “Control of ethanol production and monitoring of membrane performance by mass-spectrometric gas analysis in the coupled fermentation-pervaporation of whey permeate,” App. Microbiol. Biotechnol. 40(4),470-476 (1993); T. Ikegami, H. Yanagishita, D. Kitamoto, K. Karaya, T. Nakane, H. Matsuda, N. Koura and T. Sano, “Production of Highly Concentrated Ethanol in a Coupled Fermentation/Pervaporation Process using Silicate Membranes,” Biotechnol. Tech. 11(12), 921-924 (1997)).

We have chosen membrane pervaporation. There are many well-developed, stable, highly selective and permeable membranes available for the continuous removal of ethanol from the fermentation process. Y. S. Jeong, W. R. Vieth and T. Matsuura, “Transport and Kinetics in Sandwiched Membrane Bioreactors,” Biotechnol. Prog. 7, 130-139 (1991); D. J. O'Brien and J. C. Craig Jr., “Ethanol production in a continuous fermentation/membrane pervaporation system,” Appl. Microbiol. Biotechnol. 44(6), 699-704 (1996); S. H. Yuk, S. H. Cho, and H. B. Lee, “Composite membrane for high ethanol permeation,” Eur. Polym. J. 34(34), 499-501 (1998). Pervaporation is probably the most promising technique for the efficient continuous removal of ethanol from the fermentation mixture for the efficient breaking of the ethanol inhibition barrier. The development of pervaporation technology began in the 1950s. R. C. Binning, J. F. Jennings and E. C. Martin, “Separation of liquids by permeation through a membrane,” U.S. Pat. No. 2,985,588, issued May 23, 1961. Excellent discussion of pervaporation theory and applications (H. L. Fleming, “Consider membrane pervaporation,” Chem. Eng. Prog. 88(7), 46-52 (1992); K. Belafi-Bako, A. Kabiri-Badr, N. Dormo and L. Gubicza, “Pervaporation and its applications as downstream or integrated process,” Hung. J. Ind. Chem. 28(3), 175-179 (2000); N. Wynn, “Pervaporation comes of age,” Chem. Eng. Prog. 97(10), 66-72 (2001)) and selective permeation of organics including ethanol (D. Beaumelle, M. Marin and H. Gibert, “Pervaporation with organophilic membranes: state of the art,” Food Bioprod. Process. 71(C2), 77-89 (1993); May-Britt Hagg, “Membranes in chemical processing. A review of applications and novel developments,” Sep. Purif. Methods 27(1), 51-168 (1998); S. K. Sikdar, J. Burckle and L. Rogut, “Separation methods for environmental technologies,” Environ. Prog. 20(1), 1-11 (2001)) are available.

Membrane separation of ethanol produced in the fermentor involves the use of a membrane that has some selectivity for a specific product (ethanol in our case) within a reaction environment with gas/liquid “sweep stream” on the non-reaction side to remove product away from the membrane surface. This approach has been used to remove inhibitory product (ethanol) in situ.

For the experimental fermentor we considered for modeling, we took into consideration the permselective membrane used by Jeong et al., 1999.

Model Variation

Instead of the 4-dimensional model described above, a 5-dimensional model was used for the second round modeling. Most of the equations are the same. A few variations are added and the E term is not used. The equation are as follows:

$$\begin{matrix}{r}_{S} = ( \frac{1}{{Y}_{SX}} ) {r}_{X} + {m}_{S} {C}_{X} & ( 1 ) \\ {r}_{X} = μ {C}_{X} & ( 2 ) \\ {r}_{e} = f ( {C}_{S} ) f ( {C}_{P} ) {C}_{e} , & ( 3 )\end{matrix}$$

where the substrate dependence function ƒ(C_{S}) is given by the Monod-type relation,

$$f ( {C}_{S} ) = \frac{{C}_{S}}{{K}_{S} + {C}_{S}} . \qquad ( 4 )$$

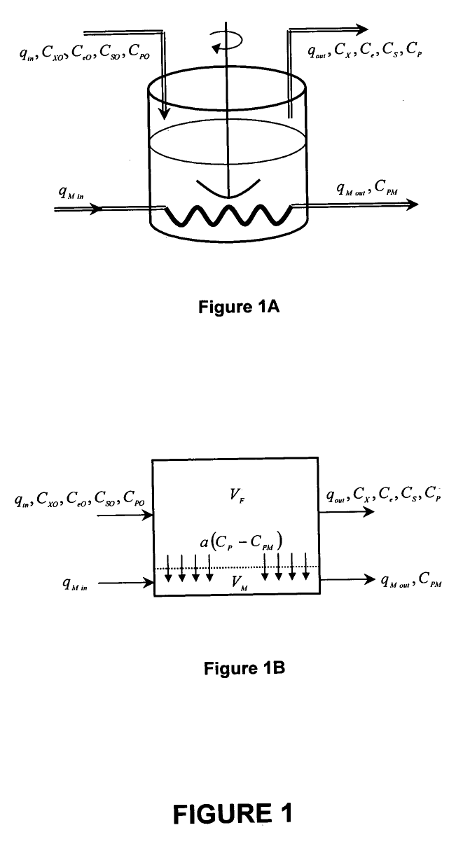

The relation between alcohol concentration C_{P} and alcohol dependence function ƒ(C_{P}) is
ƒ(C_{P})=k₁−k₂ C_{P}+k₃ (C_{P})². (5)
Based on the above, the dynamic model for the four components e, X, S and P is given by the following set of ordinary differential equations. FIGS. 1A-1B show the schematic diagrams of the fermentor and in-situ ethanol removal membrane module setup with all the flow rates and concentrations shown.

$$\begin{matrix}\frac{ⅆ {C}_{e}}{ⅆ t} = ( {k}_{1} - {k}_{2} {C}_{P} + {k}_{3} {C}_{P}^{2} ) ( \frac{{C}_{S} {C}_{e}}{{K}_{S} + {C}_{S}} ) + {D}_{in} {C}_{eO} - {D}_{out} {C}_{e} & ( 6 ) \\ \frac{ⅆ {C}_{X}}{ⅆ t} = ( \frac{{C}_{S} {C}_{e}}{{K}_{S} + {C}_{S}} ) + {D}_{in} {C}_{XO} - {D}_{out} {C}_{X} & ( 7 ) \\ \frac{ⅆ {C}_{S}}{ⅆ t} = ( \frac{- 1}{{Y}_{SX}} ) ( \frac{{C}_{S} {C}_{e}}{{K}_{S} + {C}_{S}} ) - {m}_{S} {C}_{X} + {D}_{in} {C}_{SO} - {D}_{out} {C}_{S} & ( 8 ) \\ \begin{matrix}\frac{ⅆ {C}_{P}}{ⅆ t} = ( \frac{1}{{Y}_{PX}} ) ( \frac{{C}_{S} {C}_{e}}{{K}_{S} + {C}_{S}} ) + {m}_{P} {C}_{X} + {D}_{in} {C}_{PO} - {D}_{out} {C}_{P} - \\ ( \frac{a}{{V}_{F}} ) ( {C}_{P} - {C}_{PM} )\end{matrix} & ( 9 )\end{matrix}$$

Note that equation (9) contains a term (the last term on the right hand side) for ethanol removal by membrane. The membrane differential equation is given below as equation (10). It should also be noted that

$$D = \frac{q}{V}$$

(dilution rate), where q is the constant flow rate into the fermentor, V_{F} is the active volume of fermentor, and V_{M} is the active volume inside the membrane module.

In Jobses and co-workers work, there was no membrane. In our new extended five-dimensional model, the membrane corresponds to an area of permeation A_{M}=0 corresponding to a=0 in equation (9).

The membrane-side equation (assuming perfect mixing in the membrane side, in order to simplify the preliminary analysis) is

$$\begin{matrix}\frac{ⅆ {C}_{PM}}{ⅆ t} = ( \frac{a}{{V}_{M}} ) ( {C}_{P} - {C}_{PM} ) + {D}_{M in} {C}_{PMO} - {D}_{M out} {C}_{PM} , \\ where & ( 10 ) \\ {D}_{M out} = {D}_{M in} + \frac{a ( {C}_{P} - {C}_{PM} )}{{V}_{M} ( rho )} & ( 11 ) \\ {D}_{out} = {D}_{in} - \frac{a ( {C}_{P} - {C}_{PM} )}{{V}_{F} ( rho )} & ( 12 ) \\ a = {A}_{M} P & ( 13 )\end{matrix}$$

The above-described model (consisting of ODEs (6)-(10) and algebraic equations (11)-(13)) was used to explore the different possible complex static/dynamic bifurcation behavior of this system firstly in the two-dimensional (D−C_{SO}) parameter space (for no membrane configuration) and, then later, in A_{M} parameter space (for ethanol removal membrane configuration). The model was also used to study the implications of these phenomena on physically important values of substrate conversion and ethanol yield and production rate.

The system parameters for one of the experimental runs of Jobses et al., 1985 and Jobses et al., 1986 showing oscillatory behavior were used as the base set of parameters in the second round modeling and are given in Table 3.

TABLE 3
The base set of parameters used.

| Parameter | Value |
|---|---|
| k₁ (hr⁻¹) | 16.0 |
| k₂ (m³/kg · hr) | 4.97 × 10⁻² |
| k₃ (m⁶/kg² · hr) | 3.83 × 10⁻² |
| m_{S} (kg/kg · hr) | 2.16 |
| m_{P} (kg/kg · hr) | 1.1 |
| Y_{SX} (kg/kg) | 2.44498 × 10⁻² |
| Y_{PX} (kg/kg) | 5.26315 × 10⁻² |
| K_{S} (kg/m³) | 0.5 |
| P (m/hr) | 0.1283 |
| D_{M in} (hr⁻¹) | 4.0 |
| C_{XO} (kg/m³) | 0 |
| C_{PO} (kg/m³) | 0 |
| C_{eO} (kg/m³) | 0 |
| V_{F} (m³) | 0.003 |
| V_{M} (m³) | 0.0003 |
| rho (kg/m³) | 789 |

In order to evaluate the performance of the fermentor as an alcohol producer, we calculated the conversion of substrate, the product (ethanol) yield, and its production rate according to the simple relations incorporated into the FORTRAN programs,

$$Substrate ( sugar ) conversion = {X}_{s} = \frac{{C}_{SO} {D}_{in} - {C}_{S} {D}_{out}}{{C}_{SO} {D}_{in}}$$

$$Ethanol yield = {Y}_{P} = \frac{{C}_{P} {D}_{out} {V}_{F} + {C}_{PM} {D}_{M out} {V}_{M} - {C}_{PO} {D}_{in} {V}_{F}}{{C}_{SO} {D}_{in} {V}_{F}}$$

Ethanol production rate per unit volume (kg/m³·hr) of the fermentor=

$${P}_{P} = {C}_{P} {D}_{out} + {C}_{PM} {D}_{Mout} ( \frac{{V}_{M}}{{V}_{F}} )$$

For the oscillatory and chaotic cases also, the average conversion \overline{X}_{S}, average yield \overline{Y}_{P}, and the average production rate \overline{P}_{P}, as well as the average ethanol concentration \overline{C}_{P}, were computed. These were defined as

$$\begin{matrix}{\overline{X}}_{S} = {\int }_{0}^{τ} {X}_{S} \frac{ⅆ t}{τ} , {\overline{Y}}_{P} = {\int }_{0}^{τ} {Y}_{P} \frac{ⅆ t}{τ} , \\ {\overline{P}}_{P} = {\int }_{0}^{τ} {P}_{P} \frac{ⅆ t}{τ} , {\overline{C}}_{P} = [ {\int }_{0}^{τ} {C}_{P} \frac{ⅆ t}{τ} + {\int }_{0}^{τ} {C}_{PM} \frac{ⅆ t}{τ} ] ,\end{matrix}$$

where the τ values in the periodic cases represent one period of the oscillations, and in the chaotic cases, they are taken long enough to be reasonable representation of the “average” behavior of the chaotic attractor.

For the cases without the membrane, we simply took the area of permeation to be zero (A_{M}=0) which gave us the differential equations governing the fermentor system without ethanol selective membrane.

Results and Discussion

The results are classified in two different sections:
I. fermentation without ethanol removal and
II. fermentation with continuous ethanol removal.

For the cases of “fermentation without ethanol removal” (I), the bifurcation analysis was carried out for two different bifurcation parameters—Dᵢₙ (dilution rate) and C_{SO} (influent feed substrate concentration).

For the cases of “fermentation with continuous ethanol removal” (II), the bifurcation parameter used was the area of permeation for ethanol (A_{M}) for a particular set of values of Dᵢₙ, C_{SO} and permeability of the membrane (P). Area of permeation (A_{M}) was chosen to show the effect of ethanol removal rate on substrate conversion and ethanol yield/productivity, as well as the stability of the attractors.

The reason for choosing these three bifurcation parameters (Dᵢₙ, C_{SO} and A_{M}) is that they are the easiest ones to manipulate experimentally during the operation of a laboratory or full-scale fermentor.

I. Fermentation Without Ethanol Selective Membrane (Area of Permeation A_{M}=0)

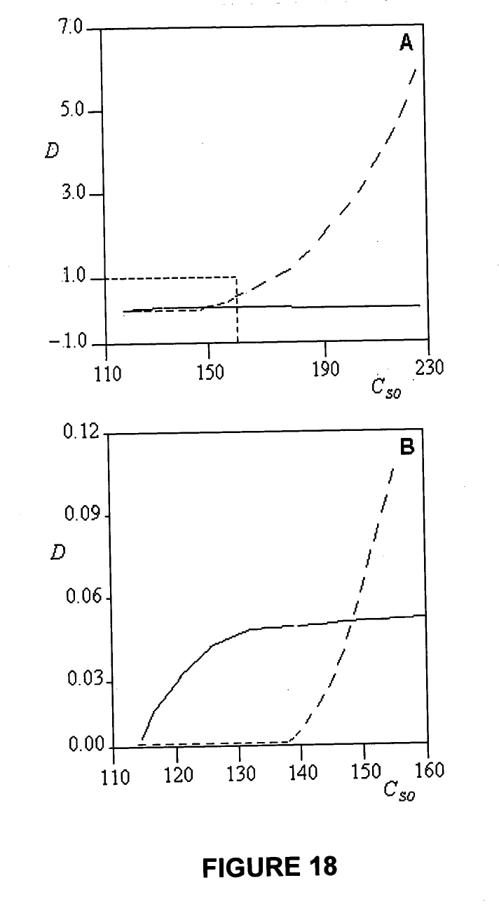

FIG. 18A is a two-parameter continuation diagram of Dᵢₙ vs. C_{SO} showing the loci of static limit points (SLPs) and HB points. One parameter bifurcation diagrams were constructed by taking fixed value of C_{SO} and constructing the Dᵢₙ bifurcation diagrams, then taking fixed values of Dᵢₙ and constructing the C_{SO} bifurcation diagrams. This diagram basically shows the corresponding location of HB and SLP points for different combinations of C_{SO} and Dᵢₙ. FIG. 18B is an enlargement of box (i) of FIG. 18A.

A) Dilution Rate Dᵢₙ as the Bifurcation Parameter
Case (A-1): C_{SO}=140 kg/m³

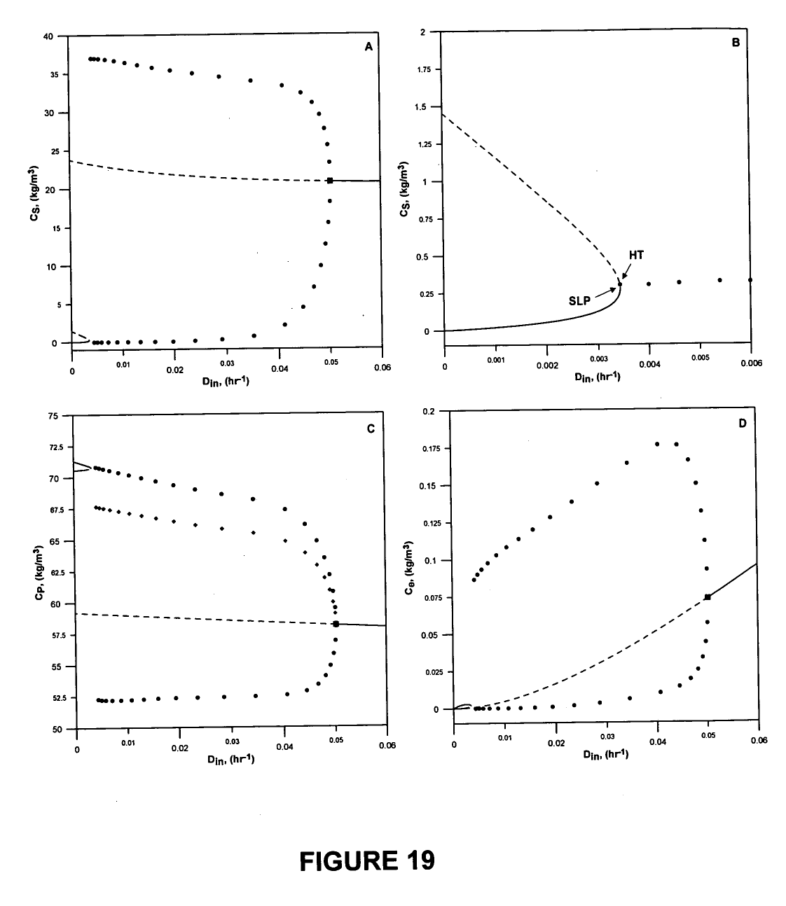

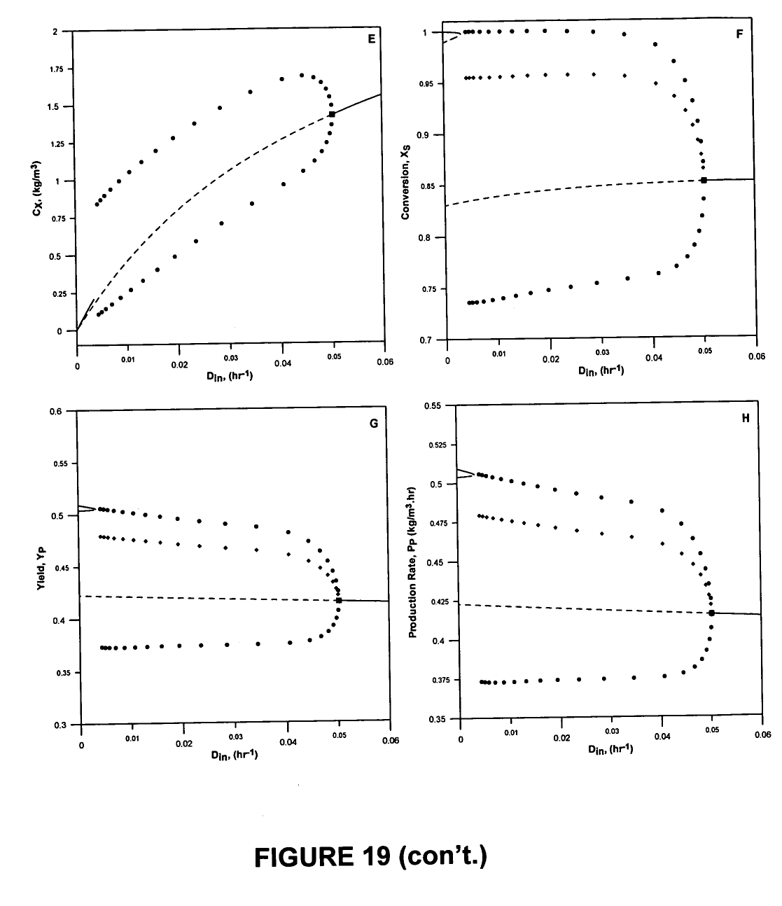

Jobses and co-workers used in their experiments this value of C_{SO} together with a dilution rate Dᵢₙ=0.022 hr⁻¹. Details for static and dynamic bifurcation behavior for this case are given in FIGS. 19A-19H, with the dilution rate Dᵢₙ as the bifurcation parameter.

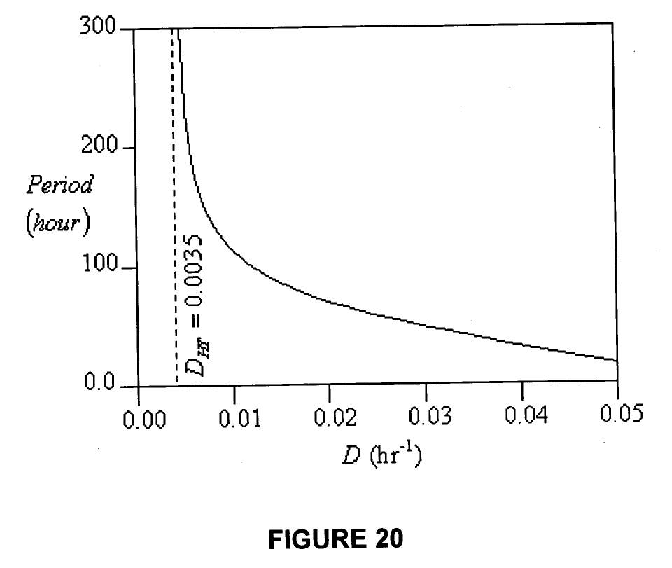

FIG. 19A shows the bifurcation diagram for substrate concentration (C_{S}). It is clear that the static bifurcation diagram is an incomplete S-shape hysteresis-type with a static limit point (SLP) at very low value of Dᵢₙ=0.0035 hr⁻¹. The dynamic bifurcation shows a Hopf bifurcation point (HB) at Dᵢₙ=0.05 hr⁻¹ with a periodic branch emanating from it. The region in the neighborhood of the SLP was enlarged in FIG. 19B. It is clear that the periodic branch emanating from HB terminates homoclinically (with infinite period) when it touches the saddle point very close to the SLP at Dᵢₙ=0.0035 hr⁻¹. FIGS. 19D and 19E are the bifurcation diagrams for the internal key component e concentration (Cₑ) and biomass concentration (C_{X}), respectively. FIG. 19C is the bifurcation diagram for the ethanol concentration (C_{P}). It is clear from FIG. 19C that the average ethanol concentrations for the periodic attractor are higher than the corresponding unstable steady states. FIGS. 19F-19H show the bifurcation diagrams for substrate conversion (X_{S}), ethanol yield (Y_{P}), and ethanol production rate (P_{P}). The average conversion (\overline{X}_{S}), yield (\overline{Y}_{P}), and production rate (\overline{P}_{P}) for periodic branch are shown as diamond-shaped points in FIGS. 19F-19H. FIG. 20 shows the period of oscillations as the periodic branch approaches homoclinical bifurcation point; the period tends to infinity indicating homoclinical termination of the periodic attractor at Dᵢₙ=0.0035 hr⁻¹. J. P. Keener, “Infinite period bifurcation and global bifurcation branches,” J. Appl. Math. 41, 127-144 (1981).

The bifurcation diagram in this case can be divided into three regions:

1. First region: It includes the range of Dᵢₙ>D_{in HB}, where D_{in HB}=0.05 hr⁻¹. In this region there is a unique stable point attractor. At D_{HB}=0.05 hr⁻¹ sugar conversion is X_{S}=0.85 and sugar concentration is C_{S}=20.997 kg/m³. C_{S} decreases (while the conversion increases) slightly with Dᵢₙ increase as shown in FIGS. 19A and 19F. The yield of ethanol is Y_{P}=0.415, and the ethanol concentration is C_{P}=58.035 kg/m³ at D_{in HB}, and they decrease slowly with the increase in Dᵢₙ as shown in FIGS. 19C and 19G. The production rate is P_{P}=2.85 kg/m³·hr at D_{in HB} and increases with the increase in Dᵢₙ (FIG. 19H).

2. Second region: This region includes the range of D_{in HB}>Dᵢₙ>D_{in HT}, (i.e., 0.05>Dᵢₙ>0.0035). In this region there is a unique periodic attractor (surrounding the unstable steady state) which starts at the HB point and terminates homoclinically at a point very close to SLP at Dᵢₙ=0.0035 hr⁻¹, as shown in FIGS. 19A-19H.

For the unstable steady state branch, the sugar concentration (C_{S}) in this region increases with the decrease of Dᵢₙ from 20.997 to 23.992 kg/m³, as shown in FIG. 19A. Similarly, the yield of ethanol (Y_{P}) increases from 0.415 to 0.425 as shown in FIG. 19G. Ethanol concentration (C_{P}) increases slightly from 58.035 to 59.235 kg/m³, and the production rate of ethanol (P_{P}) decreases from 2.85 to 0.3 kg/m³·hr as shown in FIGS. 19C and 19H, respectively.

For the periodic branch, the amplitudes of the oscillations are quite large for all state variables. The average sugar conversion varies between 0.85 and 0.878 (shown as the diamond-shaped points in FIG. 19F). The average ethanol concentration (\overline{C}_{P}) varies between 59.315 and 61.532 kg/m³ (FIG. 19C), while the average ethanol yield (\overline{Y}_{P}) values vary in this region between 0.447 and 0.42 (FIG. 19G). Similarly, the average productivity (\overline{P}_{P}) values vary between 2.9 and 0.3 kg/m³·hr as seen in FIG. 19H.

It is clear that in this region, the average of the oscillations for the periodic attractor gives (as shown in FIGS. 19C and 19F-19H) higher \overline{C}_{P}, \overline{X}_{S}, \overline{Y}_{P} and than that of the corresponding steady states, which means that the operation of the fermentor under periodic conditions in this region is not only more productive, but will also give higher ethanol concentrations by achieving better sugar conversion. Comparison between the values of the static branch and the average of the periodic branch at Dᵢₙ=0.045 hr⁻¹ shows that the percentage improvements are as follows:

| parameter | percentage improvement |
|---|---|
| \overline{C}_{P} | 9.34% |
| \overline{X}_{S} | 9.66% |
| \overline{Y}_{P} | 8.67% |
| \overline{P}_{P} | 9.84% |

3. Third region: For Dᵢₙ<0.0035 hr⁻¹, there are three steady states, two of them are unstable and only the steady state with the highest conversion is stable. The highest conversion (almost complete conversion) occurs in this region for the high conversion stable steady state, and it also gives the highest ethanol yield, which is equal to 0.51 (FIGS. 19F and 19G). On the other hand, this region has the lowest ethanol production rate (FIG. 19H) due to the low values of the dilution rate Dᵢₙ (for a given fermentor active volume, it corresponds to very low flow rate).

The upper steady state (in the multiplicity region) gives the highest ethanol concentration and yield as compared with all other steady states (including the average of periodic attractors (FIGS. 19C and 19G)).
However, it occurs at a very narrow region at very low Dᵢₙ (i.e., very low

$$( i . e . , very low \frac{{q}_{in}}{{V}_{M}} ) ,$$

thus, its productivity P_{P} (ethanol production rate per unit volume of fermentor) is drastically low (FIG. 19H). Therefore, the best production policy for ethanol concentration, yield, and productivity is the periodic attractor.

In general, there is a trade-off between concentration and productivity, which requires economic optimization study to determine the optimum Dᵢₙ. However, such an optimization study will have to take into consideration the fact that some periodic attractors have higher ethanol yield and production rate than the corresponding steady states.
Case (A-2): C_{SO}=200 kg/m³

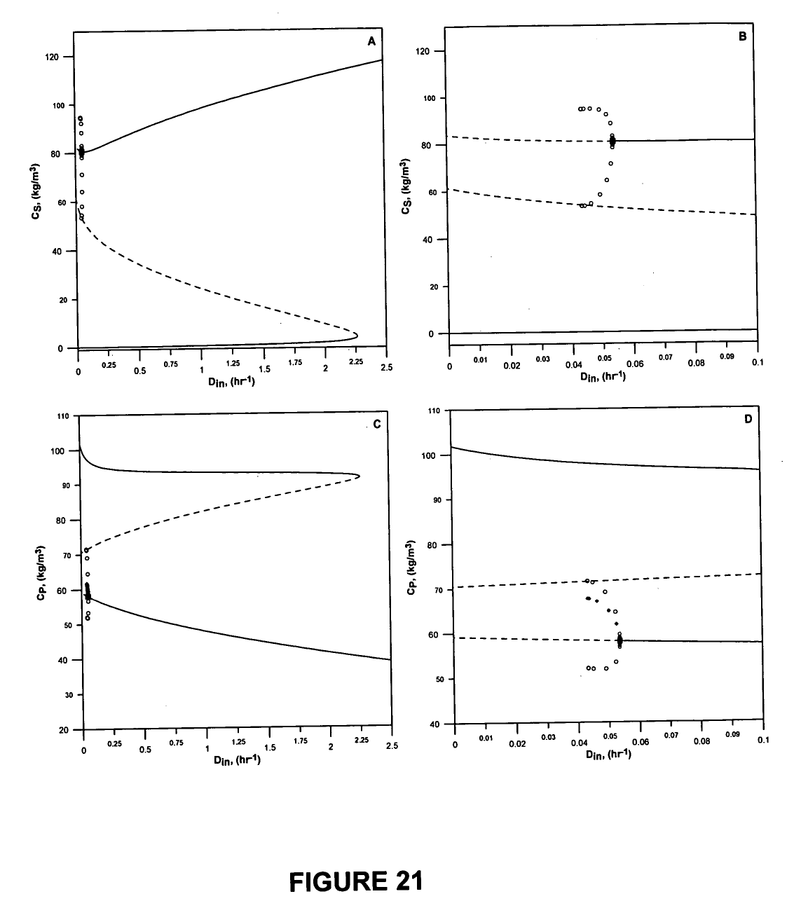

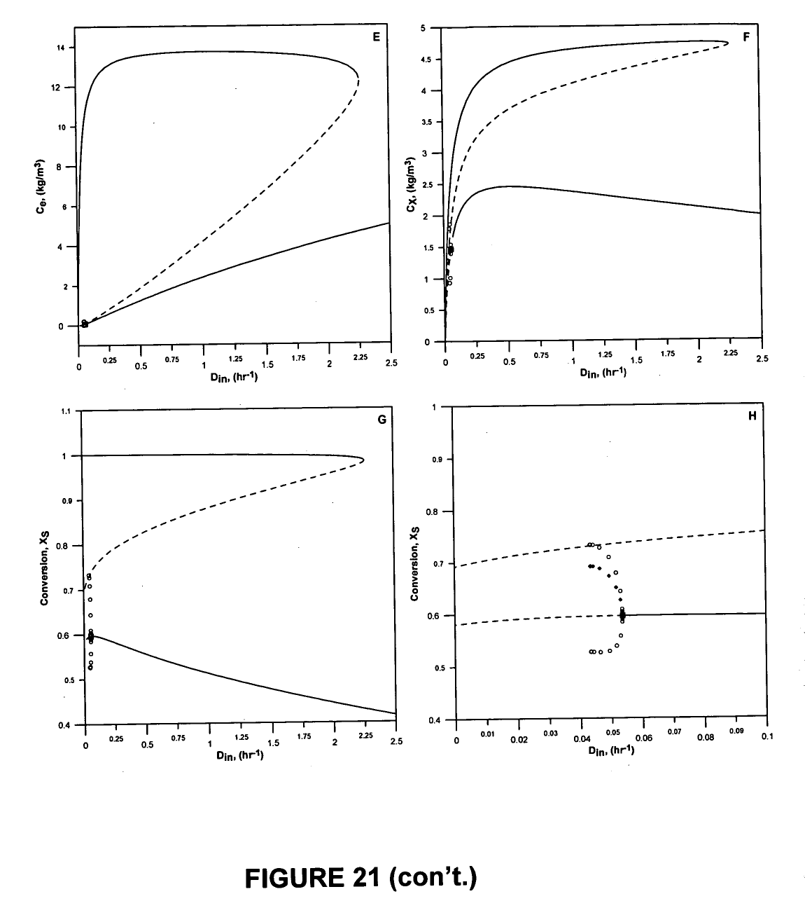

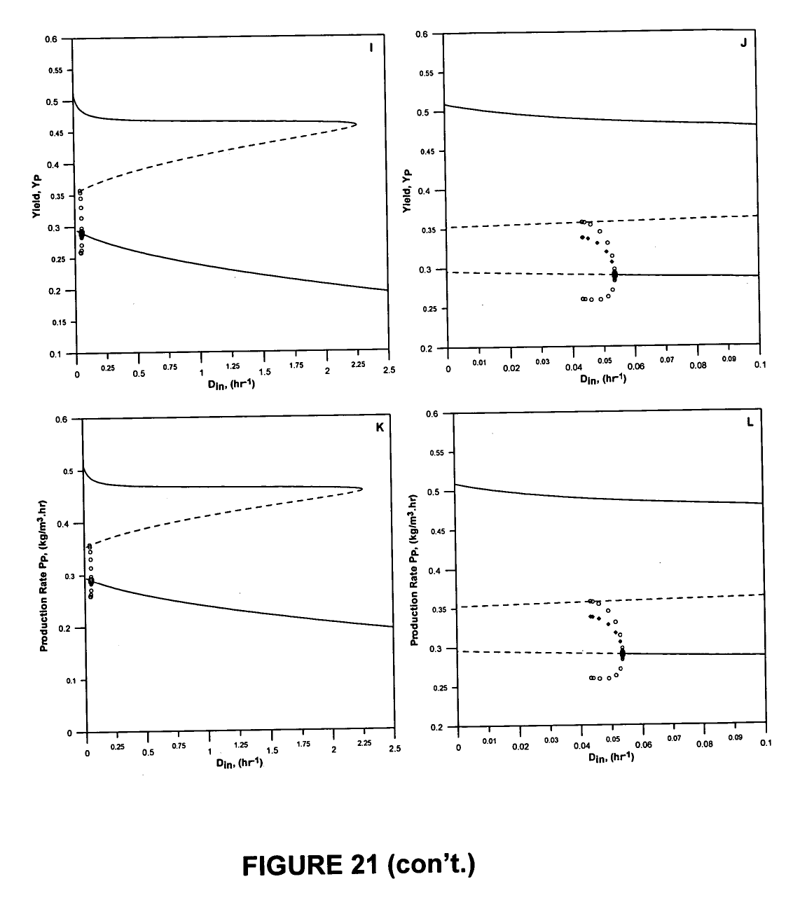

This is a case with a very high feed sugar concentration. FIGS. 21A-21F show the static and dynamic bifurcation diagrams with the dilution rate (Dᵢₙ) as the bifurcation parameter and the enlargement of the chaotic region. FIGS. 21G-21L show the bifurcation diagrams for the substrate conversion X_{S}, the ethanol production rate P_{P}, and ethanol yield Y_{P}, with the enlargement of the chaotic region. The highest conversion can be achieved on the upper branch in the range of Dᵢₙ<2.25 hr⁻¹ (almost complete conversion, FIG. 21G), Y_{P} decreases as Dᵢₙ increases (FIG. 21I) and P_{P} increases with increasing Dᵢₙ (FIG. 21K), as also seen in the previous case. This case is characterized by the existence of chaos.

The bifurcation diagram in this case can be divided into five regions:

1. First region: For Dᵢₙ>D_{in SLP}, where D_{in SLP}=2.25 hr⁻¹, unique static attractors exist on the low conversion branch. The maximum values of X_{S}=0.18 and Y_{P}=0.28 are in this region and their value decrease with increasing Dᵢₙ while the value of P_{P} increases to P_{P}=80 with increase in Dᵢₙ.

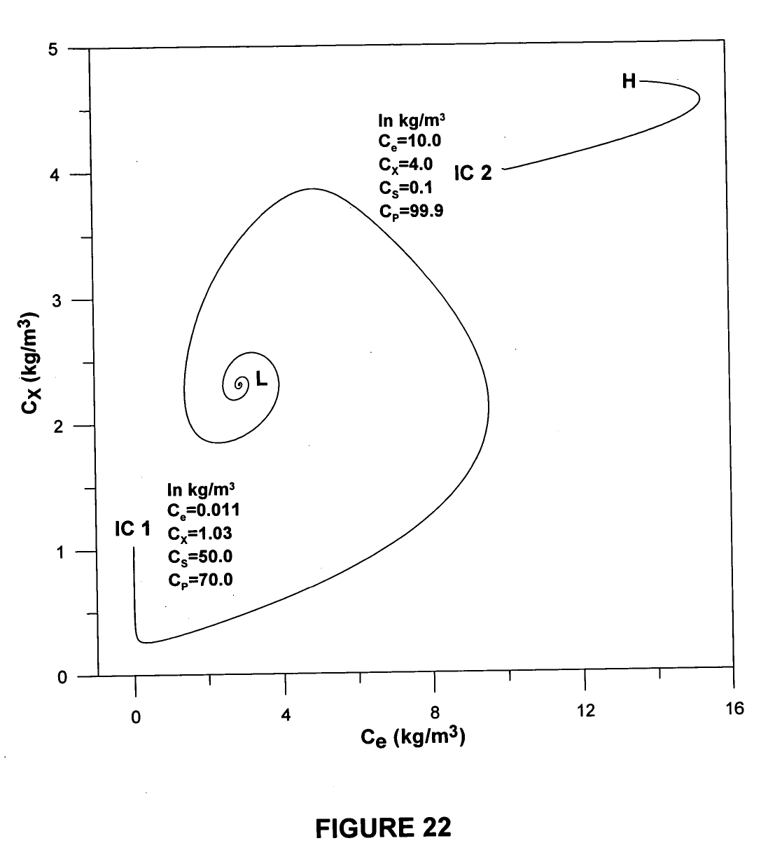

2. Second region: This region includes the range of D_{in SLP}>Dᵢₙ>D_{in HB} (i.e., 2.25>Dᵢₙ>0.054). Bistability exists where there is a very high conversion stable static branch as well as a low conversion stable static branch. Also, a saddle type unstable steady state exists (FIGS. 21A, 21C, 21E and 21F). In this region on this low conversion stable branch, the value of C_{S} increases from 89.66 to 114.22 kg/m³ while the value of C^{P} decreases from 51.79 to 40.043 kg/m³ with increasing Dᵢₙ. For the high conversion stable static branch, the value of C_{S} varies from 0.0072 to 3.15 kg/m³ while the value of C^{P} decreases slightly from 97.251 to 91.647 kg/m³ with increasing Dᵢₙ. A comparison between the values of the low and high conversion stable static branch at Dᵢₙ=1.5 hr⁻¹ shows that the high conversion branch achieves an improvement of 109.99% for X_{S}, 110.96% for Y_{P}, and 120.26% for P_{P} (FIGS. 21G-21L). The bistability is depicted in FIG. 22 where different initial conditions lead to either the low conversion or the high conversion stable steady state. This bistability behavior plays an important role in the start-up policy as a wrong start-up can eventually lead to unwanted lower conversion steady state.

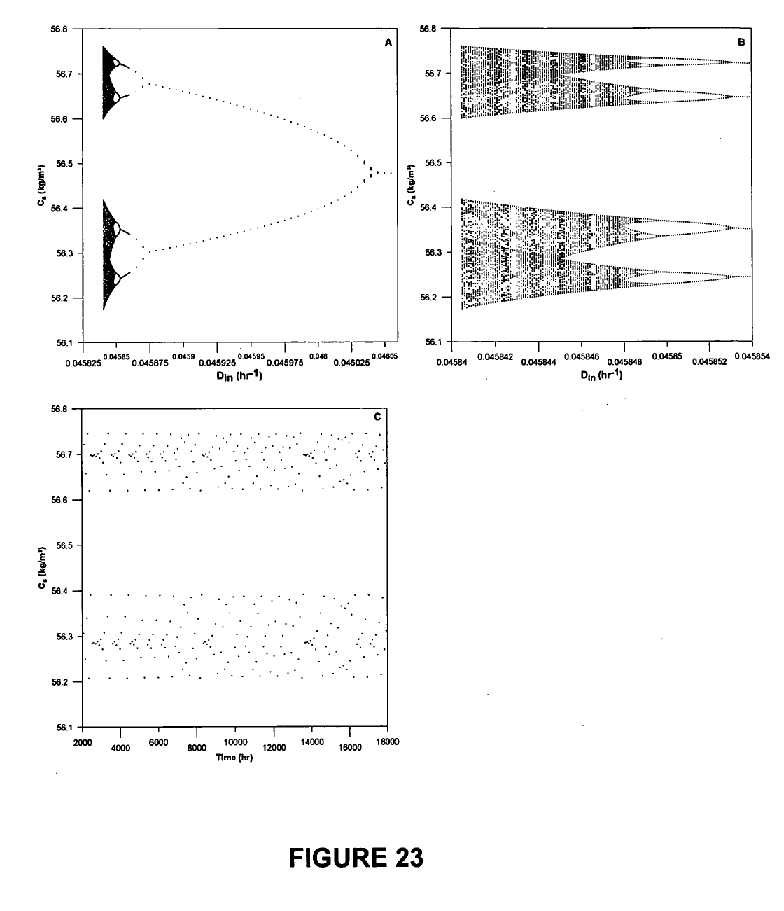

3. Third region: This region includes the range of D_{in HB}>Dᵢₙ>D_{in PD1} (i.e., 0.054>Dᵢₙ>0.04604). Bistability exists where there is a very high conversion stable static branch as well as a stable periodic branch with P1 (FIGS. 21B, 21D and 23A-23B). Comparison of the values of average of the oscillations and corresponding steady state at Dᵢₙ=0.045 hr⁻¹ (as was discussed in the previous case) shows an improvement of the following order: \overline{C}_{P} 15.434%, \overline{X}_{S} 12.01%, \overline{Y}_{P} 15.434% and \overline{P}_{P} 16.277%. But still, the average values are much less as compared to the high conversion stable static branch which has the following values at Dᵢₙ=0.045 hr⁻¹: C_{S}=0.089 kg/m³, C_{P}=93.88 kg/m³, X_{S}=0.999, Y_{P}=0.468, and P_{P}=44.265 kg/m³·hr.

4. Fourth region: This region includes the range of D_{in HT}<Dᵢₙ<D_{in PD1} (i. e., 0.045835<Dᵢₙ<0.04604). Bistability exists where there is a very high conversion stable static branch as well as a stable periodic (chaotic) branch. This region is the characteristic region of this case due to the presence of period doubling to the banded chaos (two bands); sequence is P1→P2→P4→P8→•••→ Banded Chaos, which terminates homoclinically at D_{in HT}=0.045835 hr⁻¹.

FIG. 23A is enlarged in FIG. 23B, where the two bands of chaos and the period doubling sequence are clearly shown. FIG. 23C is the return point histogram for variable C_{S} at Dᵢₙ=0.04584 hr⁻¹. The return points are taken where the trajectories cross a certain hypothetical plane (Poincaré surface, here, C_{X}=1.55 kg/m³) transversally and in the same direction.

5. Fifth region: For Dᵢₙ<0.045835 hr⁻¹, there are three steady states, two of them are unstable and only the steady state with high conversion is stable. As Dᵢₙ decreases in this region, the substrate concentration C_{S} decreases towards zero from 3.15 kg/m³ and C_{P} increases towards its maximum value of 101.848 from 91.647 kg/m³. Moreover, C_{X} and Cₑ (unlike in the previous cases where Cₑ showed a non-monotonic behavior in this range) decrease continuously towards zero (FIGS. 21E and 21F). In this region, we have the highest conversion (almost complete conversion) and highest ethanol yield which is equal to 0.509, as compared to the previous four regions. On the other hand, this region has the lowest ethanol production rate due the low value of dilution rate Dᵢₙ (FIGS. 21H, 21J and 21L).

It should be noted that upon increasing substrate feed concentration beyond 200 kg/m³, there is no change in the shape of chaos.

B) Feed Sugar Concentration (C_{SO}) as the Bifurcation Parameter
Case (B-1): Dilution rate Dᵢₙ=0.05 hr⁻¹

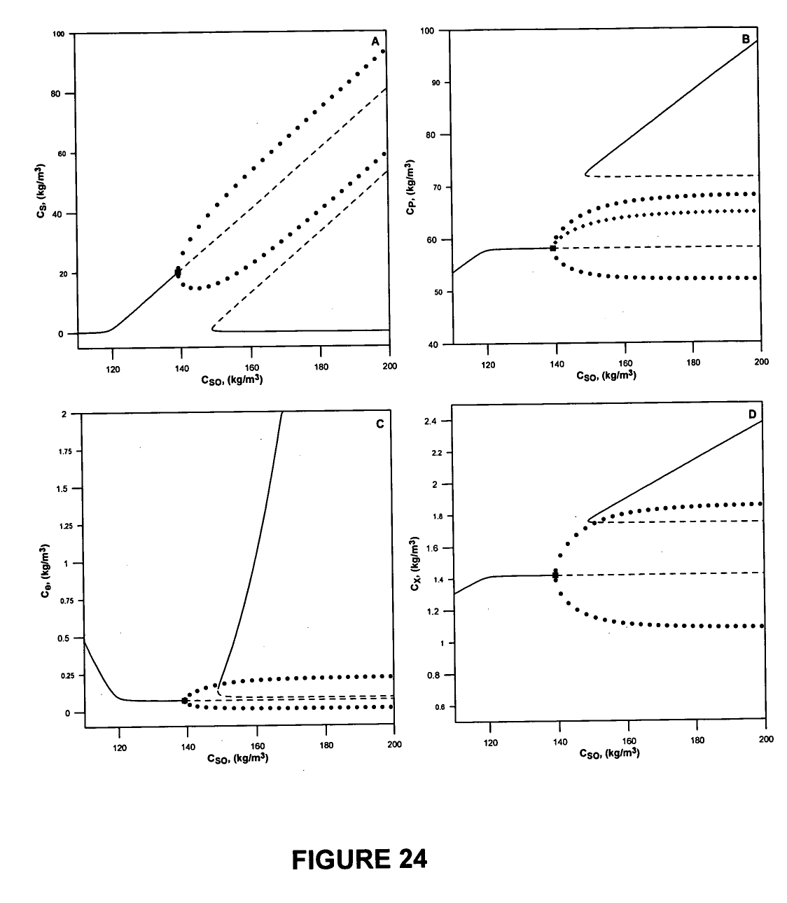

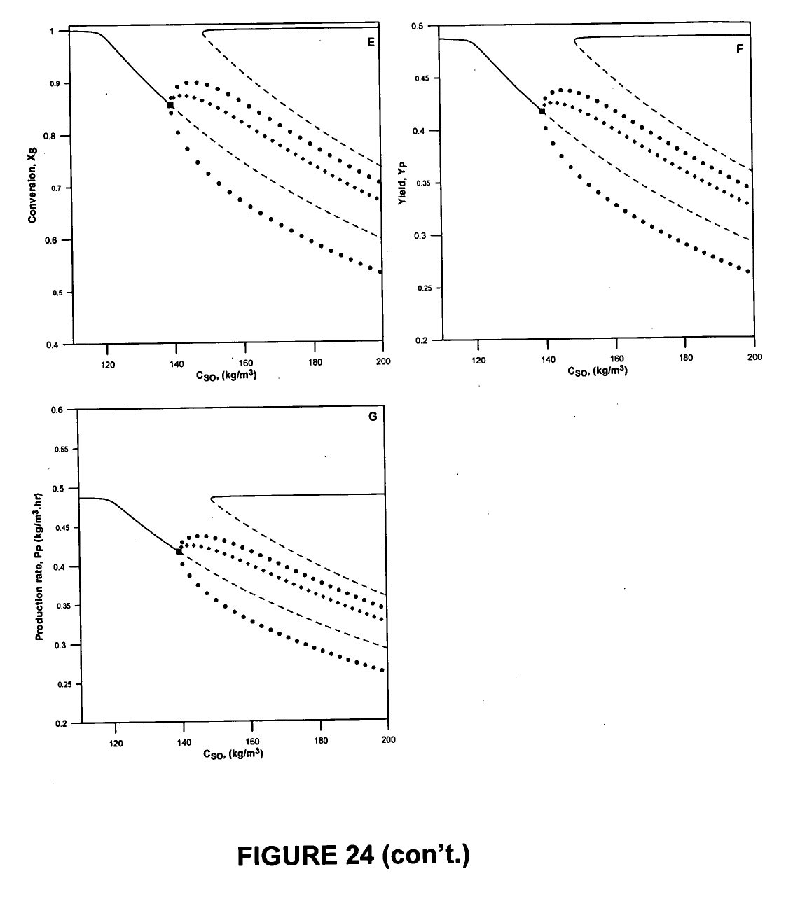

FIGS. 24A-24D show the static and dynamic bifurcation diagram with the substrate feed concentration C_{SO} as the bifurcation parameter for a fixed value of the dilution rate (Dᵢₙ=0.05 hr⁻¹). For this case, there is a static limit point (SLP) at a relatively high value of feed sugar concentration of C_{SO SLP}=148 kg/m³. The dynamic bifurcation shows a Hopf bifurcation point (HB) at C_{SO HB}=140 kg/m³ after which sustained stable oscillations are observed with increasing amplitudes with increase in C_{SO} (the periodic attractor does not terminate homoclinically within the given physically realistic range of the bifurcation parameter C_{SO}). The bifurcation diagram in this case can be divided into three regions:

1. First region: For C_{SO}>C_{SO SLP}, where C_{SO SLP}=148 kg/m³, bistability exists due to the presence of one periodic attractor, associated with one static attractor (for highest conversion branch). The stable high conversion static branch has an almost complete conversion with X_{S}=0.999 together with an almost unchanging yield value of Y_{P}=0.487. The production rate corresponding to this branch increases with C_{SO} from 3.614 to 4.875 kg/m³·hr. The stable static branch gives higher values of conversion, yield, and production rate when compared with the average of the oscillations. Corresponding to C_{SO}=180 kg/m³, it is seen that the stable static branch achieves an improvement of 35.332% for X_{S}, 35.709% for Y_{P}, and 35.672% for P_{P} over the average values of the periodic branch (FIGS. 24E-24G).

2. Second region: This region includes the range of C_{SO SLP}>C_{SO}>C_{SO HB} (i.e., 148>C_{SO}>140), where there is a unique periodic attractor with period one. Again, like the previous cases, it is observed that the average values of the oscillations are higher than the corresponding steady state values. Comparison of the values of average of the oscillations and corresponding steady state at C_{SO}=160 kg/m³ shows an improvement of the following:

| parameter | percentage improvement |
|---|---|
| \overline{C}_{P} | 9.989% |
| \overline{X}_{S} | 10.281% |
| \overline{Y}_{P} | 9.982% |
| \overline{P}_{P} | 9.989% |

(FIGS. 24B, 24E-24G).
3. Third region: This region includes the range of C_{SO}<C_{SO HB} (C_{SO}<140 kg/m³), where there is only one stable steady state (FIGS. 24A-24D). In this region, as C_{SO} increases, the substrate concentration C_{S} increases from 0.079 to 0.239 kg/m³ initially (in the range 110<C_{SO}<115.87, this is due to the fact that the sugar fed is consumed totally by the microorganisms). After this point, the value of sugar concentration increases steadily to 20.014 kg/m³ (FIG. 24A). Similar behavior is observed for C_{P}; it increases towards 58.132 from 53.583 kg/m³ (FIG. 24B). Cₑ decreases from 0.478 to 0.072 kg/m³ (FIG. 24C), and C_{X} increases from 1.307 to 1.418 kg/m³ (FIG. 24D). Conversion and ethanol yield decrease while the production rate increases with increasing C_{SO} (FIGS. 24E-24G).
Case (B-2): Dilution rate Dᵢₙ=0.045 hr⁻¹

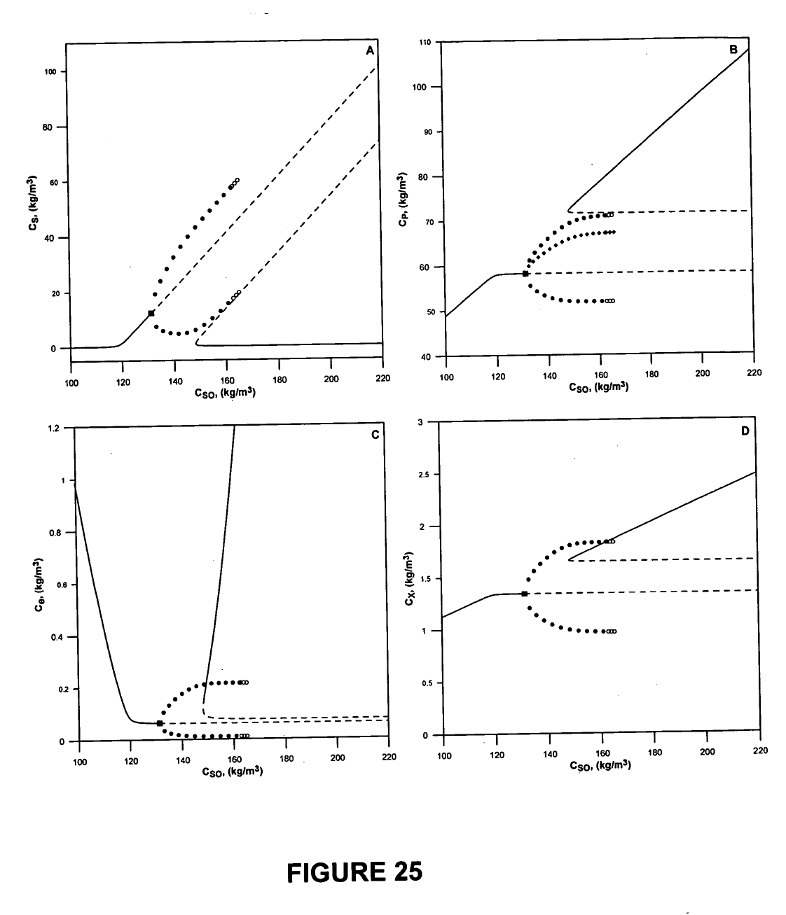

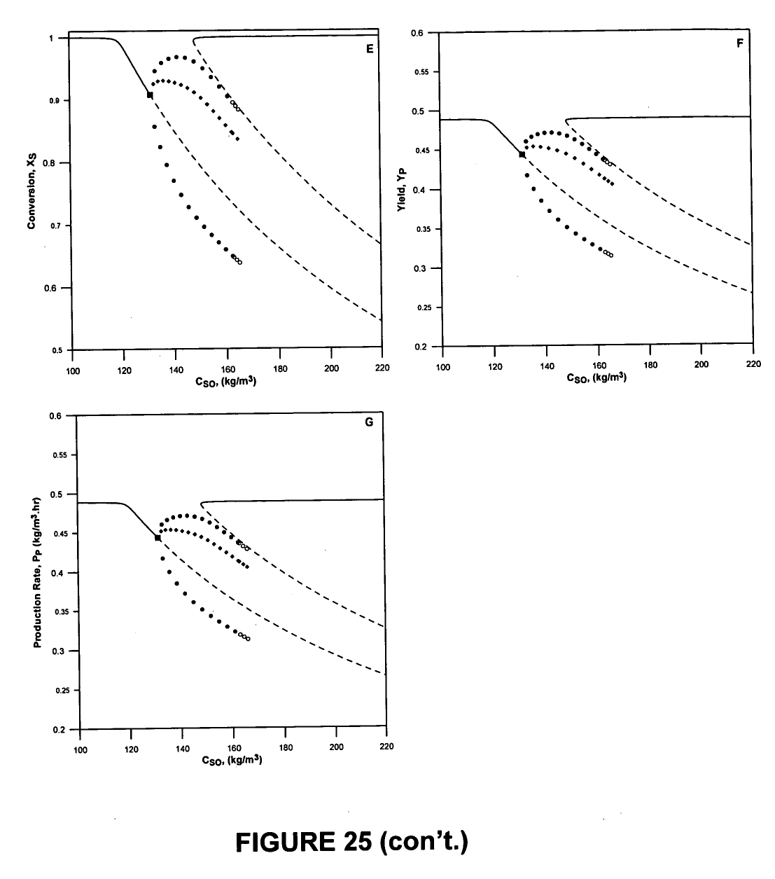

FIGS. 25A-25D show the static and dynamic bifurcation diagram with the substrate feed concentration C_{SO} as the bifurcation parameter. This case is characterized by the presence of period doubling route to banded chaos and subsequent homoclinical termination of this chaotic attractor. The bifurcation diagram in this case can be divided into the following four regions:

1. First region: For C_{SO}>C_{SO HT}, where C_{SO HT}=165.7 kg/m³, there are three steady states, two of them are unstable and only the steady state with the highest conversion is stable (FIG. 25A-25E). In this region, the value of conversion X_{S} and ethanol yield Y_{P} remain almost constant at 0.999 and 0.488, respectively (FIGS. 25E-25F). The value of production rate P_{P} increases from 3.632 to 4.84 kg/m³ hr (FIG. 25G).

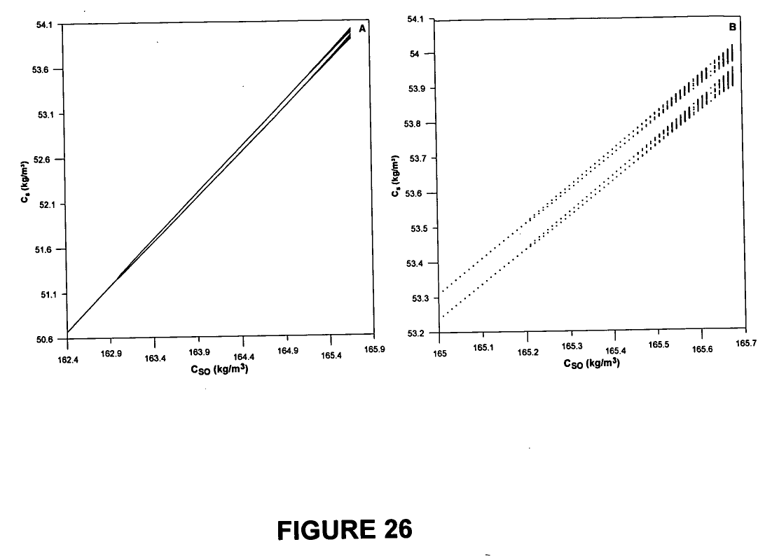

2. Second region: For C_{SO SLP}<C_{SO}<C_{SO HT} (i.e., 147<C_{SO}<165.7), bistability exists due to the presence of a periodic attractor together with a stable static attractor (the highest conversion branch). The periodic branch in this region changes its periodicity in a period doubling sequence leading to chaos, and the chaotic attractor terminates homoclinically at C_{SO HT}=165.7 kg/m³, as shown in FIGS. 25A-25D and 26A. Comparison of the values of the average of the oscillations and corresponding steady state at C_{SO}=160 kg/m³ shows an improvement of the following:

| parameter | percentage improvement |
|---|---|
| \overline{C}_{P} | 15.064% |
| \overline{X}_{S} | 15.376% |
| \overline{Y}_{P} | 15.15% |
| \overline{P}_{P} | 15.064% |

(FIGS. 25B, 25E-25G). But, it should be noted that the stable static branch still has higher values of conversion, yield, and production rate compared to the average of oscillations.
3. Third region: This region includes the range of C_{SO SLP}>C_{SO}>C_{SO HB} (i.e., 147>C_{SO}>132) where there is a unique periodic attractor with period one. In this region, the average value of the period one oscillations for ethanol concentration \overline{C}_{P} increases from 58.217 to 65.498 kg/m³ with an increase in C_{SO} (FIG. 25B). Similarly, the average conversion \overline{X}_{S} decreases from 0.907 to 0.914 (FIG. 25E), average yield \overline{Y}_{P} decreases from 0.445 to 0.443 (FIG. 25F), and average production rate \overline{P}_{P} increases from 2.619 to 2.947 kg/m³·hr (FIG. 25G).
4. Fourth region: This region includes the range of C_{SO}<C_{SO HB} (i.e., C_{SO}<132 kg/m³). There is a unique stable static attractor in this region. In this region, as C_{SO} increases, the substrate concentration C_{S} increases very slightly from 0.027 to 0.3 kg/m³ initially (in the range 100<C_{SO}<116.778, this is due to the fact that the sugar fed is consumed totally by the microorganisms). After this point, the value of sugar concentration increases steadily to 12.17 kg/m³ (FIG. 25A). Similar behavior is observed for C_{P}; it increases towards 58.271 from 48.696 kg/m³ (FIG. 25B). Cₑ decreases from 0.986 to 0.062 kg/m³ (FIG. 25C), and C_{X} increases from 1.12 to 1.34 kg/m³ (FIG. 25D). Conversion and ethanol yield decrease while as the production rate increases with increase in C_{SO} (FIG. 25E-25G).

FIG. 26A is a one-dimensional Poincare bifurcation diagram for the state variable C_{S} with C_{SO} as the bifurcation parameter which shows the period doubling route to chaos. FIG. 26A is enlarged in FIG. 26B, where the two bands of chaos and the period doubling sequence are clearly shown.

II. Fermentation with Ethanol Selective Membrane

C) Bifurcation Analysis Using Area of Permeation (A_{M}) as Bifurcation Parameter

Bifurcation analysis of the 4-dimensional system (without continuous ethanol removal) (above) was carried out based on two different bifurcation parameters, namely, dilution rate (Dᵢₙ, hr⁻¹) and feed sugar concentration (C_{SO}, kg/m³). To improve the productivity and yield, continuous removal of ethanol was then incorporated into the analysis.

A bifurcation study was carried out for such a system having the area of permeation (A_{M}, m²) as the bifurcation parameter. A_{M} was chosen as the bifurcation parameter because the membrane module used for ethanol removal can be easily modified to change the area of permeation, leading to a change in the total permeation rate of ethanol across the membrane. Furthermore, change in area gives a good visualization of how the multiplicity (and, hence, chaotic or complex attractors) give way to a stable unique steady state with relatively high production rate. Thus, the membrane acts as a controller (or stabilizer) which reduces and eventually eliminates the chaotic and oscillatory steady states (or instabilities).

Bifurcation analysis was done for two different cases having fixed values of C_{SO} and Dᵢₙ. The two new cases correspond to the cases (A-1) and (A-2) above.
Case (C-1): C_{SO}=140 kg/m³ and Dᵢₙ=0.02 hr⁻¹

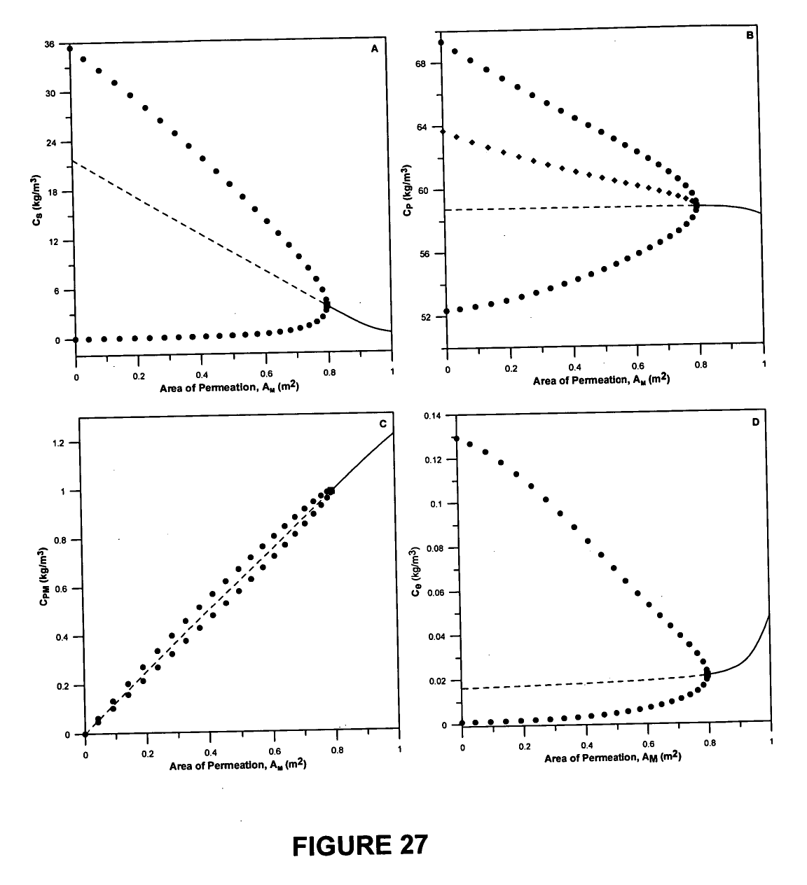

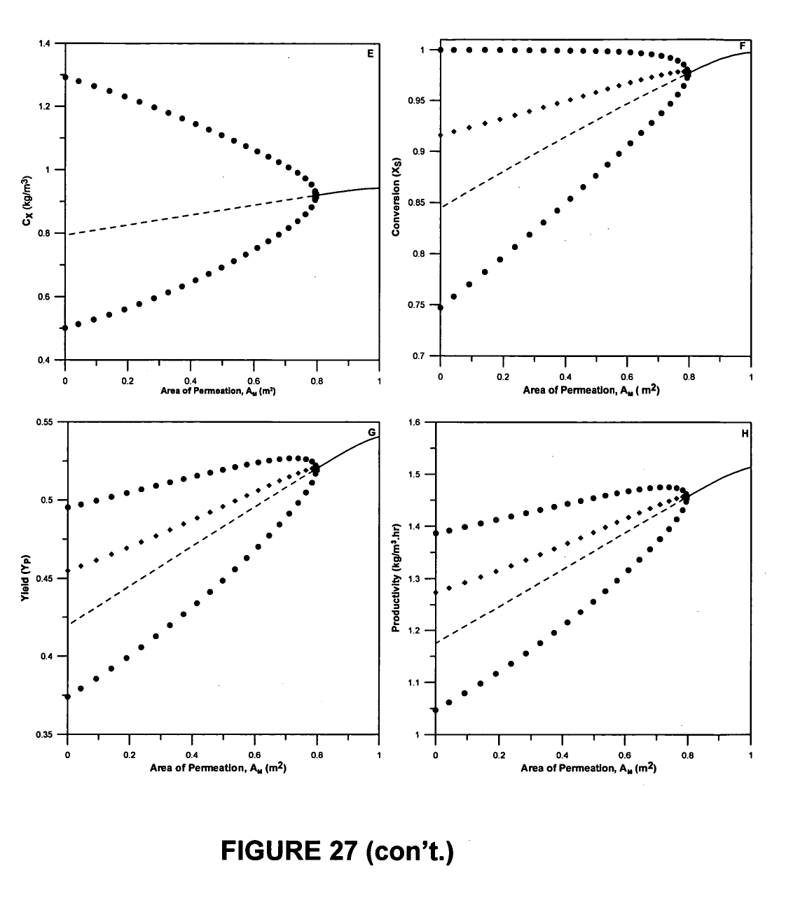

This case has a feed sugar concentration C_{SO} equal to 140 kg/m³ and a dilution rate Dᵢₙ equal to 0.02 hr⁻¹. This case corresponds to the case (A-1) above. The bifurcation diagrams for C_{S}, C_{P}, C_{PM}, Cₑ, and C_{X} are shown in FIGS. 27A-27E where area of permeation (A_{M}) is the bifurcation parameter. It can be observed that for A_{M}=0 (which corresponds to the case with no continuous ethanol removal), there is one stable periodic attractor surrounding an unstable static steady state. It is also seen that the dynamic bifurcation shows a Hopf bifurcation point (HB) at about A_{M}=0.8 m².

The complete bifurcation diagram in this case can be divided into two regions:

1. First region: This region includes the range of A_{M}>A_{M HB} (A_{M HB}=0.8 m²). In this region, there is only one unique stable steady state, i.e., where the value of C_{S} decreases from 3.8 kg/m³ to almost zero with an increase in the bifurcation parameter (FIG. 27A); correspondingly, the value of C_{P} remains almost constant at about 58.7 kg/m³ and then slightly decreases to 58.2 kg/m³ (FIG. 27B) while the value of C_{PM} increases from 0.96 kg/m³ to 1.21 kg/m³ (FIG. 27C). The decrease in C_{P} and corresponding increase in C_{PM} is due the ethanol produced continuously permeating across the membrane to be swept away by the sweep liquid. Cₑ and C_{X} also show increase in their values in this range (FIGS. 27D and 27E). In the same region with an increase in A_{M}, conversion X_{S} increases from 0.97 to 0.997 (FIG. 27F); yield increases from 0.515 to 0.537 (FIG. 27G); and the productivity increases from 1.46 to 1.52 kg/m³·hr (FIG. 27H). It is observed that increasing the area of permeation beyond 1.0 m² does not effect the conversion (which is almost equal to 1.0).

2. Second region: It includes the range of A_{M}<A_{M HB} (A_{M HB}=0.8 m²). In this region, there is a stable periodic attractor (surrounding the unstable steady state) with increasing amplitude of oscillation with decrease in A_{M}, as shown in FIGS. 27A-27H. It is observed that the unstable steady state ethanol concentration (C_{P}) remains almost constant at about 58.7 kg/m³ despite the increase in the area of permeation due to the ethanol produced permeating across the membrane, thus, leading to an increase in membrane side ethanol concentration (C_{PM}). The characteristic feature of this region is the average ethanol concentration, conversion, yield, and productivity (\overline{C}_{P}, \overline{X}_{S}, \overline{Y}_{P}, and \overline{P}_{P}, as shown in FIGS. 27C, 27F-27H) are higher than the corresponding unstable steady state. Comparison between the values of the static branch and the average of the periodic branch at A_{M}=0.2 m² shows that the percentage improvements are as follows:

| parameter | percentage improvement |
|---|---|
| \overline{C}_{P} | 5.91% |
| \overline{X}_{S} | 9.31% |
| \overline{Y}_{P} | 5.41% |
| \overline{P}_{P} | 5.36% |

FIGS. 27F-27H show the effect of increase in area of permeation to the ethanol production rate (P_{P}), ethanol yield (Y_{P}), and the substrate conversion (X_{S}). It is evident that the average values for the oscillatory attractor are greater than the corresponding values attained by static attractor (as shown by the diamond-shaped points in FIGS. 27F-27H). It can be safely concluded that operating the fermentor at the oscillatory state will eventually give a better ethanol production rate, yield, and conversion.

Another important conclusion is that the membrane acts as a controller for the fermentation process. As seen in FIGS. 27A-27H, with the increase in the area of permeation (leading to increase in removal rate of ethanol), the amplitude of the periodic attractor decreases. Further increase in the area of permeation finally leads to complete elimination of oscillations, thus, stabilizing the fermentation process.
Case (C-2): C_{SO}=200 kg/m³ and Dᵢₙ=0.04584 hr⁻¹

This case corresponds to case A-2 where a chaotic attractor is present. For the investigation with area of permeation to be the bifurcation parameter, the value of C_{SO} and Dᵢₙ were taken such that for a case of A_{M}=0.0 m², there is a chaotic attractor.

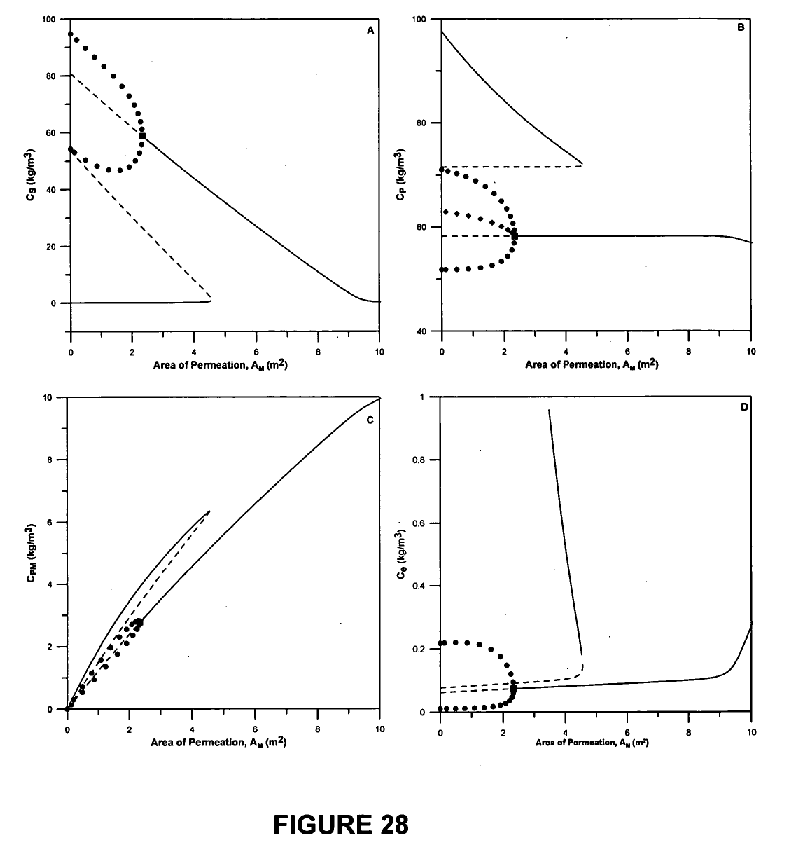

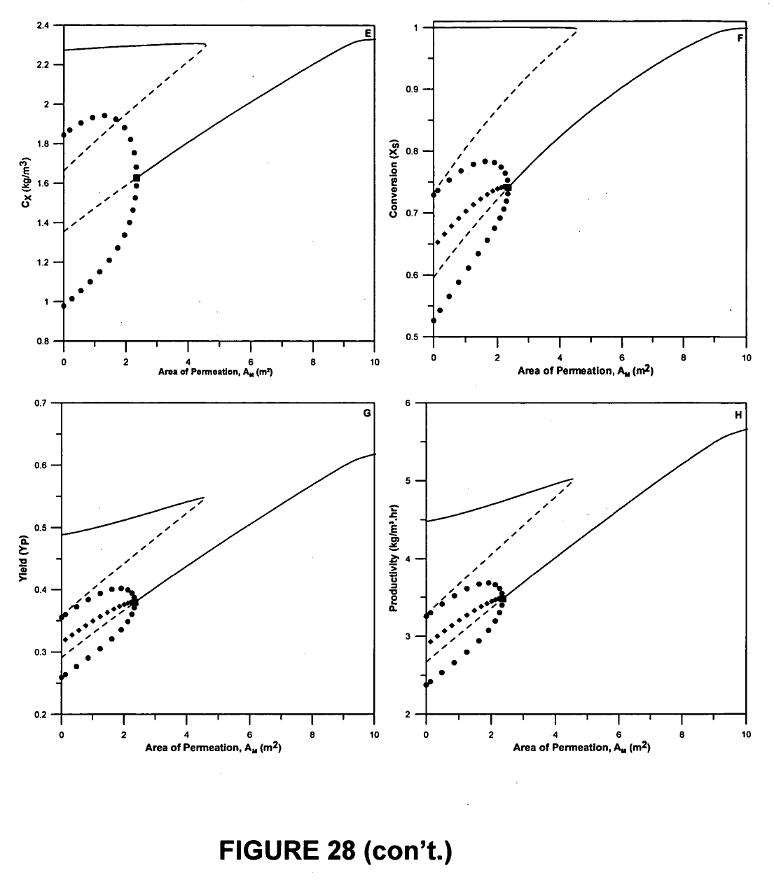

For this case, there is a static limit point at A_{M SLP}=4.556 m² and a Hopf bifurcation point at A_{M HB}=2.34686 m², as seen in FIGS. 28A-28E.

The bifurcation diagram can be divided into three regions:

1. First region: For A_{M}>A_{M SLP} (i.e., A_{M}>4.556 m²), there is a unique stable static attractor where, with an increase in A_{M}, C_{S} decreases from 39.2 to 0.27 kg/m³ (FIG. 28A); C_{P} remains almost constant at 58.24 kg/m³ (FIG. 28B) due to the ethanol produced permeating to the sweep liquid, thus, causing the C_{PM} value increase from 5.12 to 9.95 kg/m³ (FIG. 28C). Values of Cₑ and C_{X} increase with an increase in A_{M} (FIGS. 28D-28E). As expected, with an increase in A_{M}, conversion increases from 0.84 until it reaches almost complete conversion at about A_{M}=9.08 m² (FIG. 28F). Yield increases from 0.45 to 0.62 (FIG. 28G), and the productivity increases from 4.18 to 5.66 kg/m³·hr (FIG. 28H).

2. Second region: This is the range between A_{M HB}<A_{M}<A_{M SLP} (i.e., 2.34686<A_{M}<4.556). In this region there are multiple steady states (FIGS. 28A-28E). Bistability exists with a very high conversion (almost complete conversion), high yield and high productivity stable steady state co-exists with a lower conversion stable static attractor (FIGS. 28F-28H). This lower or moderate conversion stable static steady state has increasing values of conversion, yield, and productivity with increasing A_{M}. Physical significance of this region plays an important role during the start-up policy of the fermentor, as the lower conversion steady state needs to be avoided.

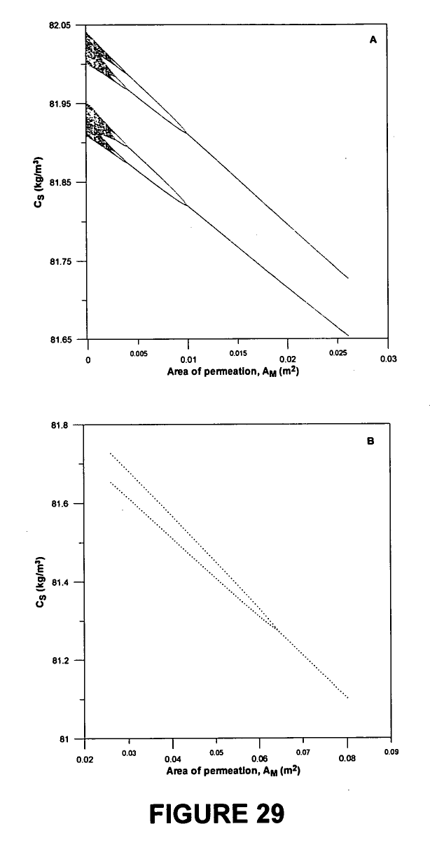

3. Third region: This region has the values of A_{M}<A_{M HB} (i.e., A_{M}<2.34686 m²). In this region there are multiple steady states, one is a stable periodic attractor surrounding an unstable static attractor, and the other is a stable static state branch (FIGS. 28A-28E). With an increase in A_{M}, this chaotic branch stabilizes to give a stable periodic attractor of periodicity one, as shown in the one dimensional Poincaré diagram (FIG. 29). It is seen that at A_{M}=0 (which corresponds to case A-1) we have two-banded chaos. This two-banded chaos loses its chaotic behavior by period halfing route with increase in A_{M} (FIG. 29A). At about A_{M}=0.0636 m², there is a period one stable attractor (FIG. 29B). Again, the average of the oscillations for the chaotic and periodic attractor gives (as shown in FIGS. 28C and 28F-28H) higher \overline{C}_{P}, \overline{X}_{S}, \overline{Y}_{P}, and \overline{P}_{P} than that of the corresponding steady states. Comparison between the values of the static branch and the average of the periodic branch at A_{M}=1.0 m² shows that the percentage improvements are:

| parameter | percentage improvement |
|---|---|
| \overline{C}_{P} | 6.32% |
| \overline{X}_{S} | 6.02% |
| \overline{Y}_{P} | 5.99% |
| \overline{P}_{P} | 5.99% |

For the region of A_{M}>A_{M SLP}, there is only one high conversion (complete conversion) stable static steady state present. But for A_{M}<A_{M SLP}, two stable steady states exist; one of which is high conversion stable steady state while the other one is an oscillatory state where the conversion, production rate, and yield increase with increase in area of permeation (where the average of oscillatory state is higher than corresponding unstable steady state), finally giving rise to a unique stable steady state with high conversion.

This reconfirms that the membrane (which results in continuous removal of ethanol from the fermentation broth) acts as a controller for the process. The oscillations are reduced and finally eliminated, thus, stabilizing the process (FIGS. 28A-28H and 29).

Another important observation is that the values of sugar conversion, ethanol yield, and productivity drop for certain values of A_{M} (4.556<A_{M}<9.1 for X_{S}, 4.556<A_{M}<6.6 for Y_{P}, and 4.556<A_{M}<7.5 for P_{P}) as the value of A_{M} is increased beyond A_{M SLP} (FIGS. 28F-28H). Thus, it can be inferred that increasing the area of permeation A_{M} can lead to lower/inferior conversion, yield, and productivity within a certain range of A_{M}. Physical significance of this finding is important while designing an experimental/industrial membrane fermentor, as this inferior conversion and yield/productivity region should be avoided.

Conclusions and Recommendations

The investigation revealed the rich static and dynamic bifurcation behavior of this five-dimensional system, which includes bistability, incomplete period doubling cascade, period doubling to banded chaos, and homoclinical (infinite period) bifurcation for periodic as well as chaotic attractors. Special emphasis was given to the implication of these phenomena on the sugar conversion, ethanol yield, and production rate of the fermentation process.

It is well known from dynamical system theory that these experimentally observed (and mathematically simulated) oscillations must start and end at certain critical points. The bifurcation parameters chosen in this investigation were the dilution rate (Dᵢₙ), feed sugar concentration (C_{SO}), and area of permeation (A_{M}). This was not only because of their importance, but also because they are the easiest to manipulate in the experimental setup.

Two parameters continuation diagrams (TPCD) were constructed for the loci of static limit points (SLP) and Hopf bifurcation (HB) points, with Dᵢₙ and C_{SO} as the two bifurcation parameters. Vertical and horizontal sections were taken on the TPCD at chosen values of Dᵢₙ and C_{SO}, and one-parameter bifurcation diagrams were constructed for all system variables as well as conversion, ethanol concentration, and ethanol production rate.

With Dᵢₙ as bifurcation parameter, the periodic branch emanates from a Hopf bifurcation point and terminates homoclinically. Table 4 reveals the location of the Hopf bifurcation points, the homoclinical termination point, the static limit point, and the type of the periodic attractor before the homoclinical termination with respect to the dilution rate in both cases.

TABLE 4
Conclusion table for different cases investigated.
Case A

| | | | | Type of periodic attractor |
|---|---|---|---|---|
| C_{SO} | D_{in HB} | D_{in SLP} | D_{in HT} | before homoclinical termination |
| (kg/m³) | (hr⁻¹) | (hr⁻¹) | (hr⁻¹) | (HT) |
| 140 | 5.00 × 10⁻² | 3.60 × 10⁻³ | 3.50 × 10⁻³ | Period I |
| 200 | 5.20 × 10⁻² | 2.25 | 4.5835 × 10⁻² | Developed Banded Chaos |

Case B

| Dᵢₙ | C_{SO HB} | C_{SO SLP} | C_{SO HT} | |
|---|---|---|---|---|
| (hr⁻¹) | (kg/m³) | (kg/m³) | (kg/m³) | |
| 0.05 | 140.0 | 148.0 | — | No HT |
| 0.045 | 132.0 | 147.0 | 165.7 | Developed Banded Chaos |

Case C

| C_{SO} | Dᵢₙ | A_{M HB} | A_{M SLP} | |
|---|---|---|---|---|
| (kg/m³) | (hr⁻¹) | (m²) | (m²) | |
| 140.0 | 0.02 | 0.8 | — | No HT |
| 200.0 | 0.04584 | 2.34686 | 4.556 | No HT |

As shown in Table 4, when C_{SO} increases (with Dᵢₙ as the bifurcation parameter), the positions of HB and SLP move to the right (increasing Dᵢₙ), but the speed of movement of SLP is greater than that of HB. This phenomenon prevents the formation of ordinary fully developed chaos because the distance between the HB point (where the periodic branch emanates from) and the saddle (where the periodic branch terminates at) is not sufficient to produce fully developed chaos. The same observation is true if C_{SO} is the bifurcation parameter. While using A_{M} as the bifurcation parameter, no homoclinical termination is observed as the increase in A_{M} stabilizes the periodic and chaotic attractors leading to the elimination of the fluctuations.

The results are fundamentally and practically important. They can be summarized in the following points:
1. The system showed static bifurcation (multiplicity of the steady state) over a wide range of parameters.
2. In the simplest cases, a HB point existed on one of the static branches, and the periodic branch emanating from it terminated homoclinically at an infinite period bifurcation (HT point) when the periodic attractor touched the saddle type steady state in the multiplicity region.
3. In more complex cases, the periodic branch showed an incomplete period doubling sequence which did not develop into chaos because the higher periodicity attractors touched the saddle type steady state and terminated homoclinically before it had completed the well-known Feigenbaum period doubling sequence to chaos.
4. In other more complex cases, the period doubling sequence completed its route to chaos, giving a region of chaotic behavior.
5. The chaotic region showed banded chaos that does not develop into fully developed chaos because before that happens, the chaotic attractor touches the saddle-type steady state and terminates homoclinically (crisis) leaving only static states (fixed point attractors).
6. Analysis of the periodic and chaotic regions shows that in these regions the average sugar conversion, ethanol yield and production rate of the periodic and chaotic attractors can be higher than their corresponding steady state values.
7. Using a membrane fermentor, sugar conversion, ethanol yield, and productivity increase as the ethanol inhibition barrier is overcome.
8. Membranes acts as stabilizing controllers for the fermentor, thus, removing the instabilities.
9. There exists a region where ethanol yield/productivity fall with the increase in area of permeation.

Experimental verification of these findings using an experimental fermentor with and without ethanol selective membrane was performed. See Examples.

The invention includes an ethanol fermentor operating in the chaotic region to improve product yield and productivity over fermentors operating at “optimum” steady states. Chaotic conditions are controllable (and the yield can be optimized) through controlling various system parameters, such as dilution rate and substrate feed concentration. Effectively produces fuel ethanol from a wide variety of sugars, including difficult to ferment sugars produced by the hydrolysis of cellulosic materials (e.g., xylose, arabinose, etc.). Addition of an ethanol selective membrane removes ethanol and increases yield (presence of ethanol causes reaction inhibition). Membranes for ethanol removal act like a controller, exhibiting a favorable stabilizing effect on the system. Such a bioreactor could have different configurations, such as continuous stirred tank (CSTR) or immobilized packed bed (IPB) fermentor configurations.

Nomenclature Used

S=Substrate (Sugar)
P=Product (Ethanol)
X=Biomass (Microorganisms)
e=Internal Key Compound (Related to X)
F=Fermentor
M=Membrane
Cᵢ=concentration of component i (kg/m³)
D=dilution rate (hr⁻¹)
E=fraction of component e in biomass (kg e/kg X)
k₁=empirical constant (hr⁻¹)
k₂=empirical constant (m³/kg·hr)
k₃=empirical constant (m⁶/kg²·hr)
K_{S}=Monod constant (kg/m³)
m_{S}=maintenance factor based on substrate requirement (kg/kg·hr)
m_{P}=maintenance factor based on product formation (kg/kg·hr)
rᵢ=production rate of component i (kg/m³·hr)
P_{P}=production rate of ethanol (kg/m³·hr or kg/hr)
\overline{P}_{P}=average production rate of ethanol (kg/m³·hr or kg/hr)
X_{S}=substrate conversion
\overline{X}_{S}=average substrate conversion
Y_{P}=yield of product (ethanol)
\overline{Y}_{P}=average yield of product (ethanol)
Y_{SX}=yield factor of biomass on substrate (kg/kg)
Y_{PX}=yield factor of biomass on product (kg/kg)
μ=specific growth rate (hr⁻¹)
τ=period of oscillation (hr)
e0=influent key component of biomass
P0=influent product (ethanol) to fermentor
S0=influent substrate (sugar)
X0=influent biomass
IP=infinite period
HB=Hopf bifurcation
HT=homoclinical termination
PD=period doubling
PDi=the i^{th} period doubling point
Pi=periodicity i of the periodic orbit
SLP=static limit point
TPCD=two parameter continuation diagram
   A. Biocatalysts, Compositions, and Equipment

Various microorganisms can be used in the bioreactor systems of the present invention. Commercial natural or genetically-modified (or recombinant) organisms can be used. Newly isolated natural or genetically-modified organisms can be produced and used according to standard methods known in the art. The choice of microorganism(s) to use in the system can be decided by one of ordinary skill in the art using conventional techniques. The amount of culture to use can be determined by one of ordinary skill in the art.

There are a variety of microorganisms used for the fermentation of ethanol. For example, conventional strains, such as yeasts (S. cerevisiae, S. uvarum, etc.) and bacteria (Z. mobilis, C. thermocellum, etc.), can be used. Also, recombinant strains (genetically engineered) have been used. Example strains which are capable of fermenting “difficult” sugars are recombinant Z. mobilis for xylose and arabinose fermentation developed by Zhang, et al. (U.S. Pat. No. 5,843,760) and recombinant S. cerevisiae strain 1400 (pLNH33) for glucose and xylose fermentation developed by Dr. Nancy Ho and Dr. George Tsao (U.S. Pat. No. 5,789,210).

Alternatively, enzymes or other biological catalysts can be used apart from whole organisms. Choice of biocatalyst can be determined by one of ordinary skill in the art.

Various substrates can be used in a method of the invention. Substrates that can be used for the fermentation of ethanol, for example, include various “simple” sugars (e.g., glucose, xylose, etc.) and “difficult sugars” (e.g., arabinose, xylose). The choice of substrate can be determined by one of ordinary skill in the art.

Additional compositions can be added to the system such as micronutrients, co-substrates, and the like. One of ordinary skill in the art can determine appropriate compositions that can be added to the bioreactor system.

Products (and by-products) produced by the invention are dependent on the microorganisms, substrates, and reaction conditions used in the process and are known or readily determined by one of ordinary skill in the art.

Equipment for use in the present invention includes conventional bioreactor and related equipment. Bioreactors include, for example, fermentors. The bioreactors can take a variety of configurations, for example, CSTRs or various bed type reactors, such as a fixed/packed bed of immobilized organisms. One of ordinary skill in the art can determine the appropriate equipment for the desired system and application.

The bioreactors are preferably operated in a continuous mode.

Associated equipment of the bioreactor typically includes pumps, tanks, and the like.

An apparatus of the invention can include a fermentor and a product specific membrane. The apparatus can further include a control system.

B. Methods

It has been found that a method of the current invention can provide average conversion, yield or productivity higher than conventional systems/methods by using periodic/chaotic attractors.

A preferred method of the invention utilizes removal of an inhibitory product to improve conversion, yield, and/or productivity.

Membrane introduction to a method of the invention can minimize product inhibition thereby increasing conversion, yield, and productivity.

Inhibitory product removal, such as ethanol in the case of the preferred method, can stabilize the system thereby reducing or eliminating oscillation.

An example fermentor with continuous ethanol removal had results of ethanol production rate increased by ˜57% and conversion of sugar reaches ˜100%. Both substrate and product inhibition were overcome. In this fermentor unstable steady state and periodic/chaotic attractors subsequently become a point attractor. Since multiplicity exists, better control strategies are required. There also exists a region where membrane causes lower conversion, yield, and productivity.

A method of the invention can utilize bifurcation analysis which can lead to better control and optimization strategies for the process.

A method of the invention can use control of chaos to increase the yield and productivity, such as for ethanol.

It is believed that the techniques of the invention can be utilized spatio-temporally to improve systems as well.

C. Utility

The methods/systems/apparatuses of the present invention are expected to be useful in most any bioreactor application, for example, ethanol production, environmental applications, pharmaceutical applications, and the like.

The exploitation of the higher ethanol production under autonomous or non-autonomous periodic and chaotic operation can then be achieved on a commercial scale using this verified model as reliable design equations. The possibility of using “control of chaos” (Tamura, T., Inaba, N. and Miyamichi, J. (1999). “Mechanism for taming chaos by weak harmonic perturbations,” Phys. Rev. Letters, 83(19), 3824-3827; Ajbar, A. (2001). “Stabilization of chaotic behavior in a two-phase autocatalytic reactor,” Chaos, Solitons and Fractals, 12(5), 903-918) theory to generate attractors with higher ethanol productivity than those associated with autonomous systems will also be explored.

### EXAMPLES

The following examples are put forth so as to provide those of ordinary skill in the art with a complete disclosure and description of how the compounds, compositions, articles, devices, and/or methods described and claimed herein are made and evaluated and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Experimental Model Verification

Experimental investigation was undertaken as an extension of the bifurcation studies (described above) on a structured-unsegregated model for continuous sugar fermentation to ethanol using Zymomonas mobilis.

The above modeling utilized bifurcation analysis as a tool for evaluating the transient model of the continuous fermentation process for the production of ethanol. Bifurcation analysis utilizing the model equations was used to locate steady-state solutions, periodic solutions, and bifurcation points where the static and dynamic behavior changes drastically. Qualitative and quantitative changes were represented in the form of bifurcation diagrams. The diagrams were used to determine the static and dynamic accuracy of the model as compared to the experimental results.

The qualitative properties of a nonlinear dynamical system can change significantly as a result of small variations in model parameters, unlike the behavior in a linear dynamical system. Multiplicity of steady states, stability of steady states, onset and existence of periodic or oscillatory states, and more complex strange nonchaotic or chaotic attractors are some examples of these complex nonlinear qualitative properties.

The mathematical results are presented and then verified by laboratory experiments in the next section.

Case 1: C_{SO}=140 q/L and Dilution Rate D is Used as the Bifurcation Parameter.

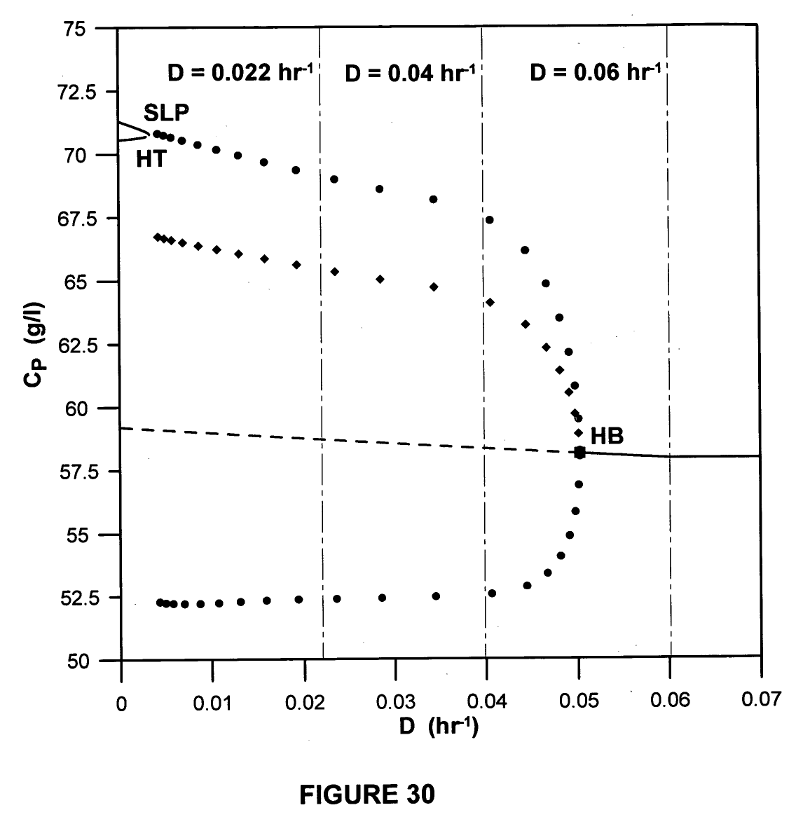

Details of the static and dynamic bifurcation behavior for this case are shown in FIG. 30, with dilution rate D as the bifurcation parameter for product concentration (C_{P}). Dotted vertical lines show the locations of the dilution rates at which the experiments were performed. It is clear that the static bifurcation diagram is an incomplete S-shaped hysteresis-type with a static limit point (SLP) at the very low value of D_{SLP}=0.0035 h⁻¹. The dynamic bifurcation shows a Hopf bifurcation (HB) at D_{HB}=0.05 h⁻¹. The periodic branch emanating from the HB terminates homoclinically (i.e., reaches a homoclinic termination, HT) when it touches the saddle point very close to the SLP at D_{HT}=0.0035 h⁻¹.

The region of interest in this case is D_{HB}>D>D_{HT}, which is characterized by a unique periodic attractor (surrounding the unique unstable steady state). It is clear that in this region the average of the oscillations for the periodic attractor gives a higher ethanol concentration than that of the corresponding steady states. The average concentrations were calculated by taking the average of the concentrations over one period of oscillation. The operation of the fermentor under periodic conditions was productive and also gave higher ethanol concentrations.

The best production policy in terms of the ethanol concentration, yield, and productivity for this case is a periodic attractor. In general, there is a tradeoff between concentration and productivity, which requires an economic optimization study to determine the optimum value of D.

Case 2: C_{SO}=200 g/L and Dilution Rate D is Used as the Bifurcation Parameter.

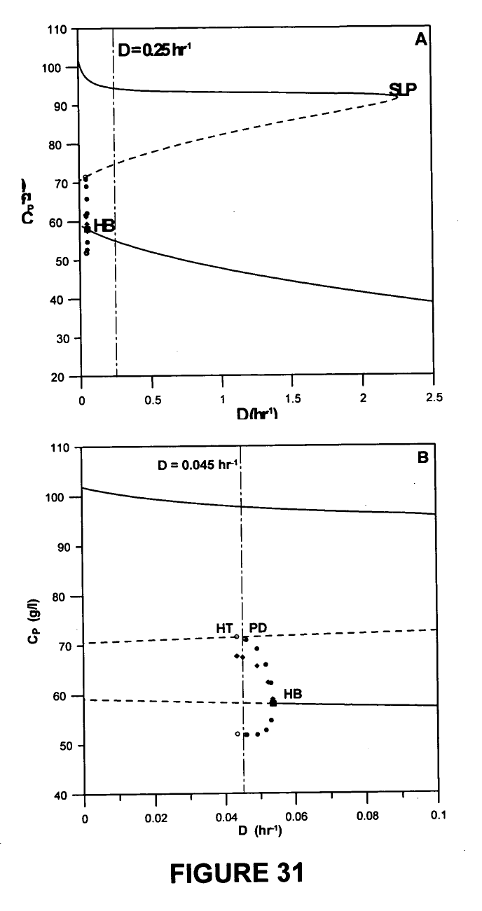

This is a case with a very high feed sugar concentration. FIG. 31 shows the static and dynamic bifurcation diagrams for this case. This case is characterized by the existence of fully developed chaos because of the period doubling to fully developed chaos (FIG. 31B); the sequence is P1→P2→P4→P8→ . . . → fully developed chaos, which terminates homoclinically at D_{HT}=0.045835 h⁻¹.

Experiments were carried out at two dilution rates (0.25 and 0.045 hr⁻¹), identified by dotted vertical lines in FIG. 31A and 31B.

In the region that includes the range of D_{SLP}>D>D_{HB} (i.e., 2.25>D>0.054), bistability exists, with a high-conversion stable static branch (conversion values in the range 0.975-1.0), as well as a low-conversion (conversion values in the range 0.42-0.595) stable static branch (FIG. 31A). A comparison between the values of the low- and high-conversion stable static branches at D=1.5 h⁻¹ showed that the high-conversion branch achieved an improvement of 120.26% in ethanol productivity over the low-conversion branch.

In the range of D_{HB}>D>D_{PD} (i.e., 0.054>D>0.04604), bistability again exists, with a high-conversion stable static branch as well as a stable periodic attractor with periodicity 1 (FIG. 31 B).

Experimental Setup

Batch and continuous runs were conducted to experimentally verify some of the characteristics of the fermentation processes at different parameter values. Most of the experimental runs were conducted in continuous mode because the prime objective of the experiments was to verify the continuous system modeled, investigated, and analyzed above.

Microorganism and Fermentation Medium

Zymomonas mobilis strain ATCC 10988 obtained from ATCC was used for the experimental runs. The strain was kept on agar dishes containing 20 g/L glucose and 10 g/L yeast extract in a refrigerator and was transferred every 2-4 weeks. The strain was also preserved at −20° C. in Eppendoff tubes containing 15% (w/v) glycerol. The cultivation medium consisted of 50 g/L glucose, 1 g/L KH₂PO₄, 2 g/L NH₄Cl, 0.49 g/L MgSO₄.7H₂O, 5 mg/L calcium panthothenate, 5 mg/L FeSO₄.7H₂O, 7.2 mg/L ZnSO₄.7H₂O, 1.5 mg/L CaCl₂.2H₂O, 4.2 mg/L MnSO₄.H₂O, 2.0 mg/L CuSO₄.5H₂O, 1.6 mg/L CoSO₄.7H₂O, 50 mg/L NaCl, and 50 mg/L KCl.

Experimental Setup and Operation

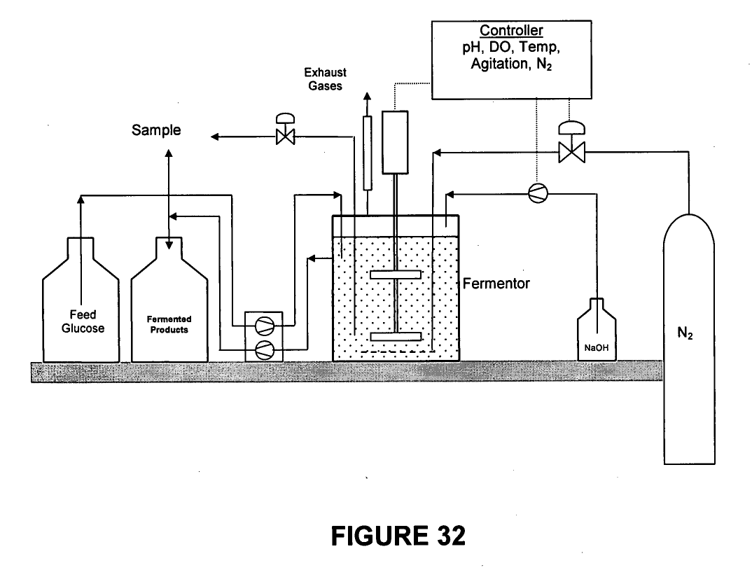

The medium of inoculum consisted of 20 g/L glucose and 10 g/L yeast extract. The cultures were seeded with 150 mL of inoculum, and the culture pH was kept at 5.0 by an automatic pH controller using 1 M NaOH. Steady states in continuous cultures were assumed to be established after 6-8 times the residence time. Samples were taken at an interval of 3 or 6 h for the continuous mode of operation. A schematic diagram of the experimental fermentor with a working volume of 2.8 L operating in continuous mode is shown in FIG. 32.

Analytical Methods

Glucose and ethanol were determined by HPLC using a Bio-Rad Aminex HPX-87H column. Glucose was also monitored with a YSI 2300 glucose/lactate analyzer (YSI Co., Yellow Springs, Ohio). The optical density of the fermentation broth was noted at a wavelength of 600 nm using a Gilford 250 spectrophotometer. The dry weight of the biomass (dry cell mass, DCM) was determined by centrifugation. The biomass was washed first with saline water and then mixed, centrifuged, washed twice with deionized water, and dried at 85° C. until reaching a constant weight.

Experimental Results and Discussion

Batch Experiments

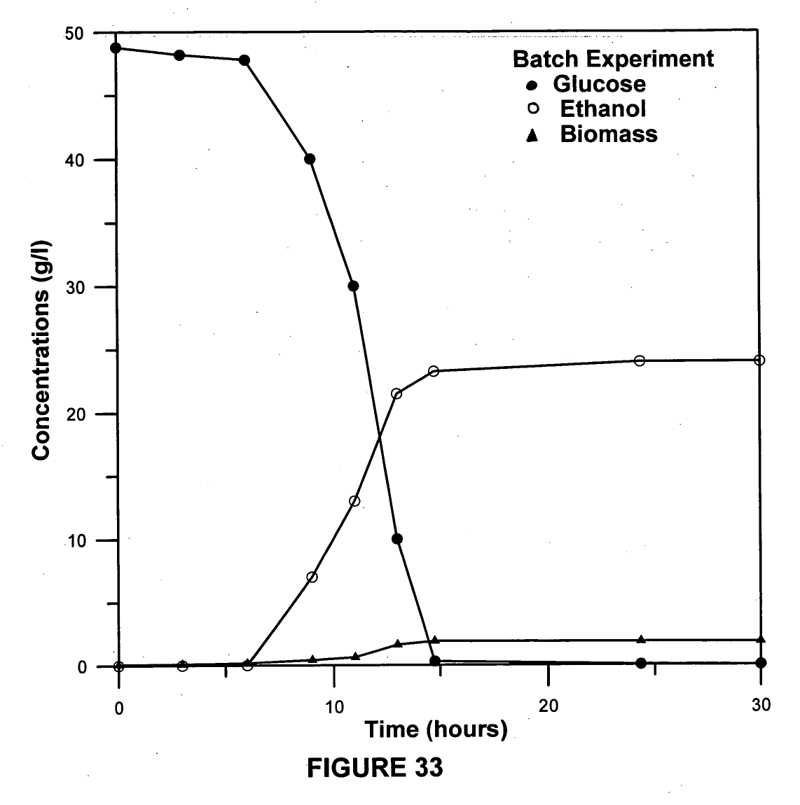

A few batch experiments were conducted for different initial sugar concentrations. The purpose of conducting the batch experiments was to formulate a growth curve that could be used to predict the inoculation time for the continuous experiments. Typical results from one of the batch fermentation runs are shown in FIG. 33. The initial glucose concentration in this batch experiment was 48.8 g/L, and the ethanol concentration was 0.002 g/L.

It can be seen in FIG. 33 that the glucose, ethanol, and biomass concentrations remain almost constant during the first 6-8 h of the batch operation. After this lag phase, an exponential phase was observed in which the biomass concentration increased sharply and, in doing so, consumed a great deal of glucose to produce ethanol. Thus, an exponential increase in the biomass and ethanol concentrations was observed while the glucose concentration dropped to almost zero. After the exponential phase, a stationary phase was observed, and it was seen that, after some time, the biomass concentration started to decrease slightly, as no more glucose was available for consumption, and growth of active microorganism stops.

Continuous Experiments

Several runs of the continuous fermentation experiments (initially starting in batch fashion and then switching to continuous mode) were conducted to verify the complex nonlinear behavior of the fermentation process discovered and explained above. No provision for ethanol removal by any means was incorporated in the experimental setup in this work.

The continuous fermentation experiments were conducted with two different feed sugar concentrations: 140 and 200 g/L. These feed concentrations correspond to cases 1 and 2, respectively, discussed above.

Feed Sugar Concentration C_{SO}=140 g/L

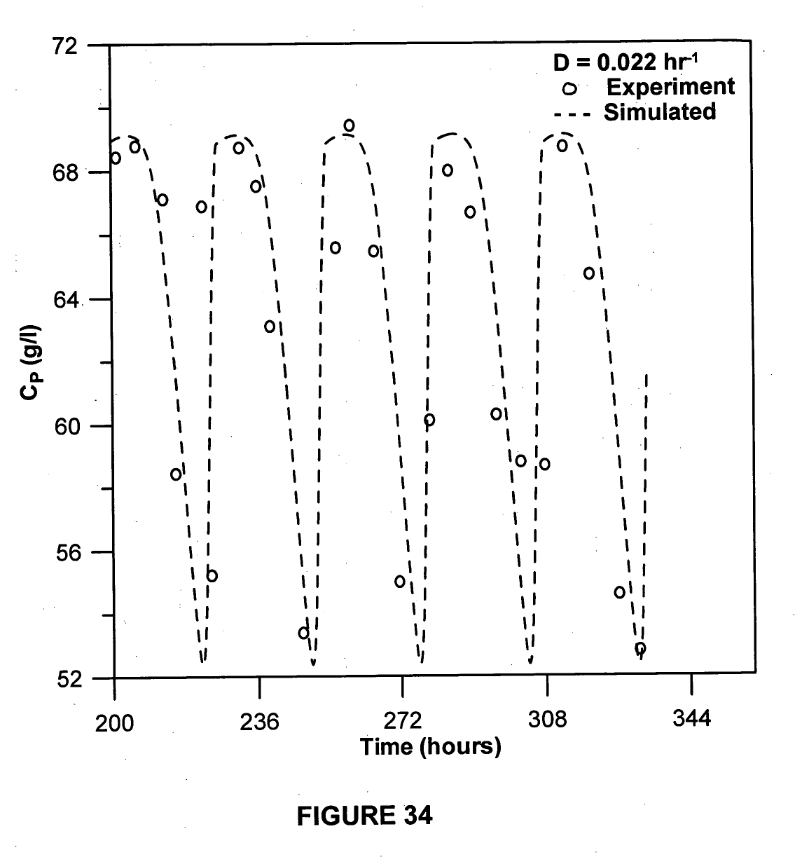

Results of the continuous runs are presented in FIGS. 34-36. These experimental runs were carried out with an inlet feed glucose concentration of 140 g/L. The aim of these experiments was the validation of the periodic behavior shown in FIG. 30.

The stream coming out of the fermentor was continuously collected in a reservoir placed in an ice bath so that any further action of microorganism with the remaining glucose was prevented. The outlet stream was collected for a certain period of time (84 h) over an ice bath and mixed well so that an analysis could be performed to determine the average concentrations of ethanol, glucose, and microorganisms.

FIGS. 34-36 show a comparison of the simulated and experimental results at three different dilution rates: 0.022, 0.04, and 0.06 h⁻¹. After the continuous-mode fermentation was started, the system was allowed to stabilize such that the initial transients were “washed out”, and then the samples were analyzed to record the data.

A dilution rate of 0.022 h⁻¹ was used for the results presented in FIG. 34 (dotted line=results of the dynamic simulation obtained from the model; small circles=experimental values). According to the nonlinear analysis above, the average ethanol concentration should be equal to 65.3 g/L. The experimental ethanol concentration over time is fairly consistent with the simulated result. The average experimental ethanol concentration was determined to be 63.89 g/L. The small difference between the average ethanol concentrations in the simulation and experimental run might be due to the fact that some of the ethanol might have escaped in vapor form from the fermentor, thus, reducing the overall ethanol concentration in the fermentation broth. Moreover, the experimental average was calculated using a finite number of points, which can exclude the maxima and minima of the oscillations.

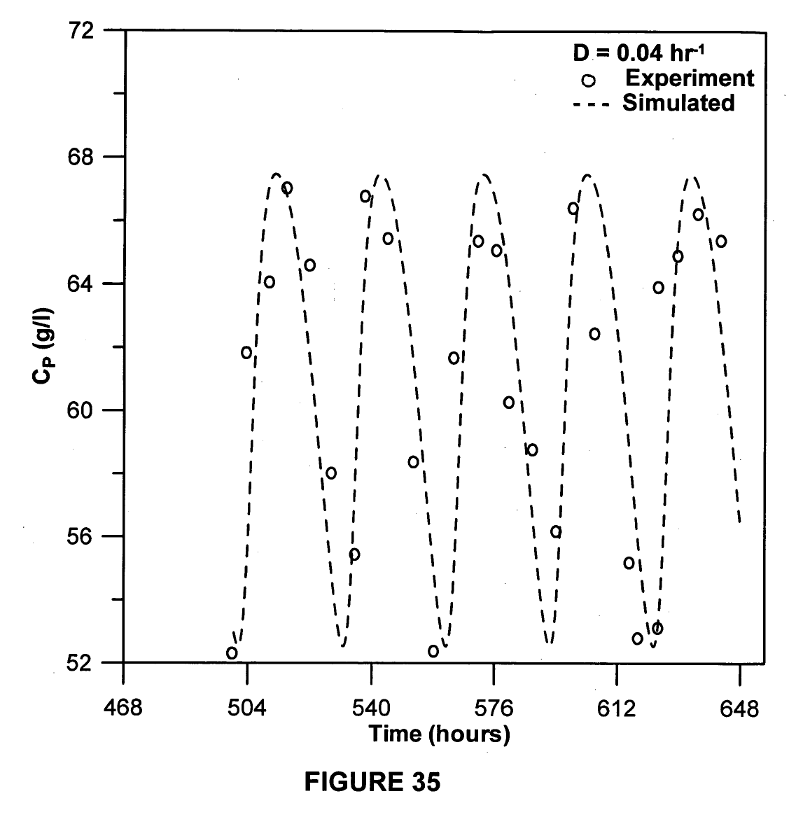

FIG. 35 shows the same data for a dilution rate of 0.04 h⁻¹, and it also corresponds to a stable periodic attractor. Again, it is observed that the experimental and simulated concentrations closely match each other. The simulated average ethanol concentration in this case was 63.4 g/L, and the experimental average ethanol concentration was slightly lower at 61.93 g/L, which might be due to the same reason mentioned above.

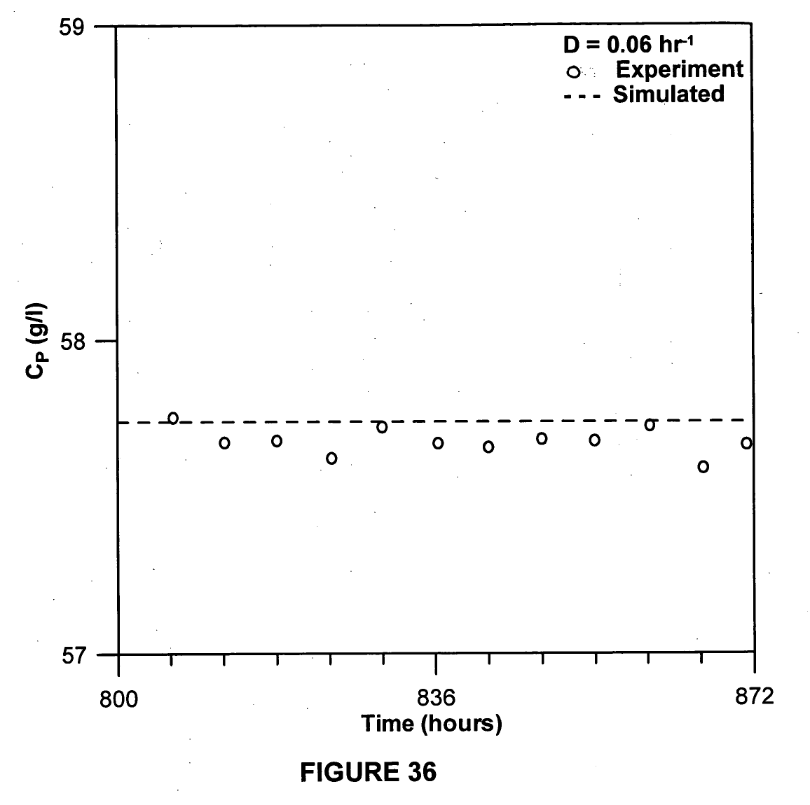

FIG. 36 depicts the system behavior at a dilution rate value of 0.06 h⁻¹. At this high dilution rate, the system had crossed the Hopf bifurcation point (FIG. 30) and had only a unique stable attractor (point attractor), which means that the state variables did not change with time. In FIG. 36 the ethanol concentration was fairly constant with very little variation over time. The average value of the ethanol concentration over a long period of time for this dilution rate was equal to 57.33 g/L, whereas the simulated value was 57.9 g/L.

Feed Sugar Concentration C_{SO}=200 g/L

For the second case, continuous fermentation experiments were carried out with the feed sugar concentration of 200 g/L. The main purpose of these experiments was to validate experimentally the existence of the multiplicity phenomenon (FIG. 31) in the model. Three different experimental runs were completed, each starting in batch mode and then being switched to continuous mode. The experiments were started in batch mode and were run to achieve certain glucose, ethanol, and microorganism concentrations to simulate different initial conditions; later, the continuous feed of pure glucose and product removal at the same flow rate were started to switch to continuous operation mode.

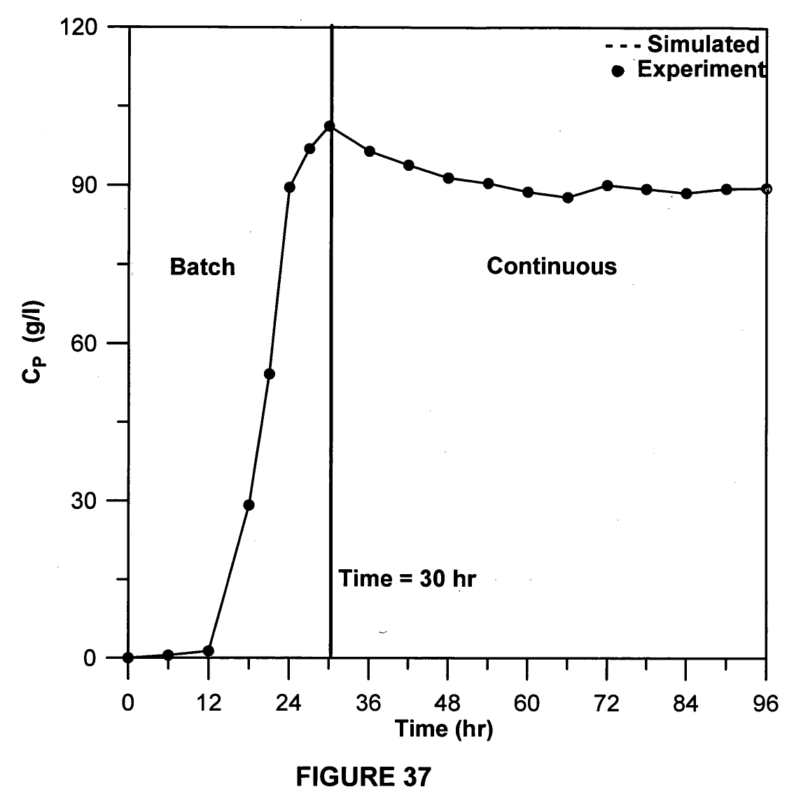

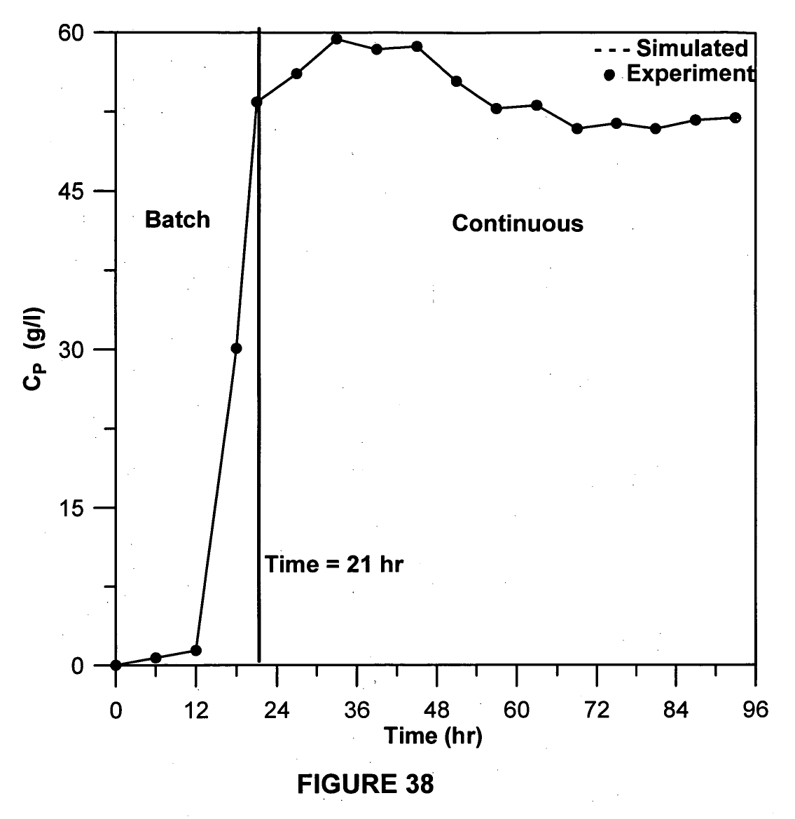

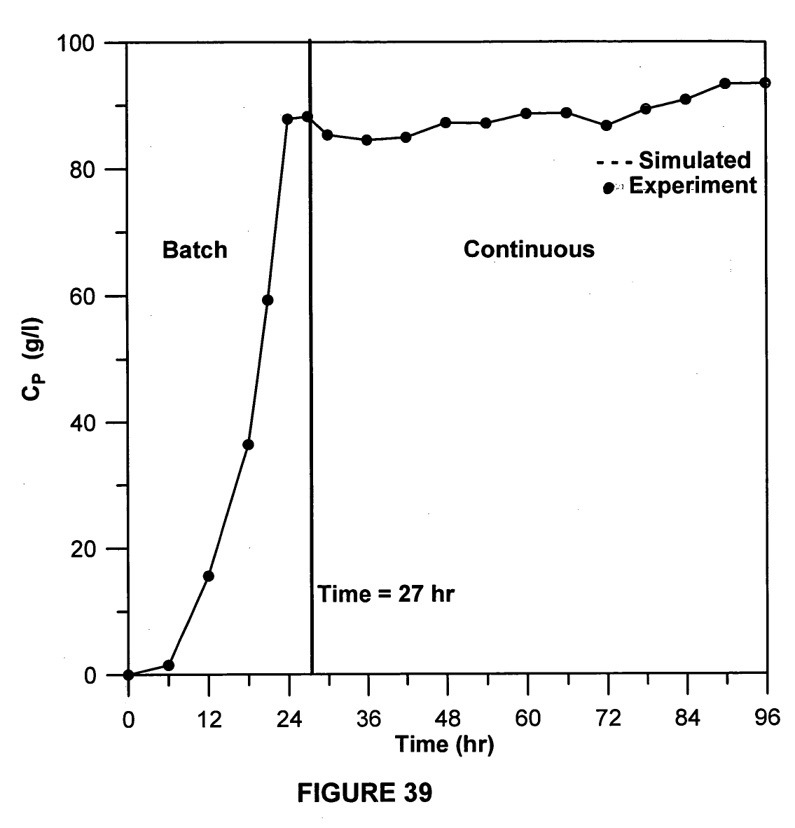

Two dilution rates were used: The first was D=0.25 h⁻¹, for which two different initial conditions were tested. It is clear that the system settled to different steady states (FIGS. 37 and 38) because of the multiplicity phenomenon occurring at this dilution rate. The second dilution rate was D=0.045 h⁻¹, for which another steady state was achieved (FIG. 39).

FIG. 37 shows the trajectory of the ethanol concentration with time. The portion on the left side of the bold vertical line depicts the batch mode of operation, and the portion on right side of line depicts the continuous mode. The experiment was run in batch mode to achieve an ethanol concentration close to the value corresponding to the high-ethanol- concentration branch of about 100 g/L, after which it was switched to continuous mode.

It is seen that the ethanol concentration decreased slightly with time and finally reaches a stable value of about 89.6 g/L. The expected value of the stable steady state for this case from the model was 95 g/L. This discrepancy can be attributed to some loss of ethanol due to evaporation and calls for further improvement in the model parameters.

Similarly to FIG. 37, FIG. 38 shows the trajectory of the ethanol concentration leading to the lower-ethanol-concentration branch. This time, the continuous operation was started when the ethanol concentration was about 54 g/L during batch operation. The ethanol concentration finally settled at a value of 51.1 g/L, which is slightly lower than the model-expected value of 55 g/L. From FIGS. 37 and 38, the multiplicity phenomenon was confirmed, as the final ethanol concentration is dependent on the initial conditions of the process for identical parameter values.

A lower dilution rate (0.045 h⁻¹) was used for FIG. 39. The lower dilution rate gave a final ethanol concentration of 93.4 g/L, whereas the simulated value was about 98 g/L. Despite the use of different initial conditions, the system could not settle at the periodic attractor as expected from the bifurcation diagram (FIG. 31B). This might be due to the fact that the region of attraction for this stable periodic attractor is very small as compared with the region of attraction of the stable steady state.

Experimental Conclusions

An extensive nonlinear investigation of the continuous fermentation process for producing ethanol from sugar was carried out. Bifurcation analysis provided insight into the possible utilization of periodic attractors to enhance the conversion, yield, and productivity of the fermentation process. Experimental verification of the mathematical investigation followed.

The continuous experiments experimental values of the state variables closely match the simulated values, thus, confirming that the simplified structured-unsegregated model is suitable for the description of the present fermentation process.

Experiments were carried out to show that a change in bifurcation parameter (dilution rate, D h⁻¹) results in sustained oscillations. Moreover, when the dilution rate is above the Hopf bifurcation value, the oscillations disappear to give a steady-state value. Experiments were also carried out to show the existence of multiple steady states (multiplicity) by starting the experiments at different initial conditions.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A method for producing a fermented product comprising:
selecting a desired fermentation process with oscillatory process characteristics;
providing a fermentor and a biocatalyst;
feeding a substrate to the fermentor;
fermenting under chaotic conditions to produce the fermented product; and
wherein the biocatalyst and substrate correspond to the desired fermentation process.

2. The method of claim 1 wherein the biocatalyst is a microorganism.

3. The method of claim 1 wherein the biocatalyst is an enzyme.

4. The method of claim 1 further comprising modeling the desired fermentation process and performing bifurcation analysis to determine the chaotic conditions.

5. The method of claim 1 wherein fermenting underchaotic conditions comprises operating the process at its periodic/chaotic attractors.

6. The method of claim 1 further comprising providing a membrane selective for the product to remove inhibition of the product.

7. The method of claim 1 further comprising separating the product from the fermentation broth using pervaporation membrane separation.

8. The method of claim 1 further comprising controlling the conditions to operate the process under chaotic conditions.

9. The method of claim 8 wherein the controlling is by control of dilution rate.

10. The method of claim 8 wherein the controlling is by control of substrate feed concentration.

11. The method of claim 8 wherein the controlling is by using a fuzzy logic controller.

12. The method of claim 1 wherein the fermentor is a continuous stirred tank reactor (CSTR).

13. The method of claim 1 wherein the fermentor is an immobilized packed bed reactor.

14. The method of claim 1 wherein the fermented product is ethanol.

15. The method of claim 1 further comprising hydrolysis of biomass to provide the substrate.

16. The method of claim 1 wherein the substrate comprises xylose and/or arabinose.

17. The method of claim 1 further comprising utilizing bifurcation analysis to determine the periodic chaotic attractors of the fermentation process.

18. A method for producing a product using a bioreactor comprising:
selecting a desired biochemical process with oscillatory process characteristics, and feeding a substrate to the bioreactor;
providing a bioreactor and a biocatalyst; and
operating the bioreactor under chaotic conditions to produce the product,
wherein the biocatalyst and substrate correspond to the biochemical process.

19. An improved method for fermentation of ethanol wherein the improvement comprises operating the fermentor under chaotic conditions.

20. The method of claim 19 further comprising providing an ethanol selective membrane.

21. A method for increasing efficiency and yield of an ethanol fermentation process relative to the same ethanol fermentation process operated at steady state conditions comprising:
selecting an ethanol fermentation process with oscillatory process characteristics; feeding a suitable substrate to the fermentor;
providing a fermentor and a biocatalyst suitable for ethanol fermentation; and
fermenting under chaotic conditions.

22. The method of claim 21 wherein the increased yield relative to a conventional process is about 100%.

23. An apparatus for fermenting ethanol comprising:
a fermentor;
a process control system capable of operating the fermentor under chaotic conditions; and
a membrane selective for ethanol.

24. The aparatus of claim 23 wherein the fermentor is a CSTR.
